(19) United States
(12) Patent Application Publication
(10) Pub. No.: US 2026/0271618 A1
(43) Pub. Date: Sep. 10, 2026

(54) MATERIALS FOR ELECTRONIC DEVICES

(71) Applicant: Merck Patent GmbH, Darmstadt (DE)

(72) Inventors: Rouven Linge, Darmstadt (DE); Elvira Montenegro, Weinheim (DE)

(21) Appl. No.: 19/650,652

(22) Filed: Apr. 17, 2026

Related U.S. Application Data
(63) Continuation of application No. PCT/EP2024/079106, filed on Oct. 16, 2024.

(30) Foreign Application Priority Data
Oct. 17, 2023 (EP) 23203979.2

(51) Int. Cl. H10K 85/60; C07D 307/91; C07D 493/04; H10K 50/15

(52) U.S. Cl. CPC H10K 85/6574; C07D 307/91; C07D 493/04; H10K 50/15

(57) ABSTRACT

A compound is provided according to a formula (I), along with its use in electronic devices, a method for its preparation, and an electronic device that includes the compound according to the formula (I)

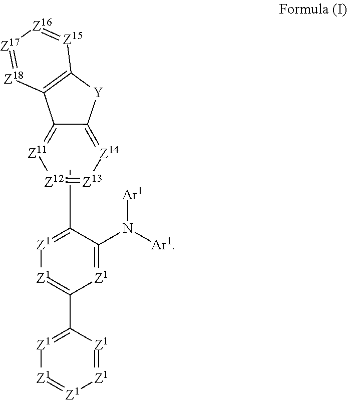

### CROSS REFERENCE TO RELATED APPLICATIONS

This application is a Continuation under 35 USC § 111(a) of International Patent Application No. PCT/EP2024/079106 filed Oct. 16, 2024, which claims priority to EP Patent Application No. 23203979.2 filed Oct. 17, 2023. The entire contents of these applications are incorporated herein by reference in their entirety.

The present application relates to aromatic amine compounds, which contain a heteroaryl group selected from aryl- or heteroaryl-substituted dibenzofuranyl and aryl- or heteroaryl-substituted dibenzothiophenyl, and a biphenyl containing moiety which has an amine substituent bonded to it. The compounds are suitable for use in electronic devices.

Electronic devices in the context of this application are understood to mean what are called organic electronic devices, which comprise organic semiconductor materials as functional materials. More particularly, these are understood to mean OLEDs (organic electroluminescent devices). The term OLEDs is understood to mean electronic devices which have one or more layers comprising organic compounds and emit light on application of electrical voltage. The construction and general principle of function of OLEDs are known to those skilled in the art.

In electronic devices, especially OLEDs, there is great interest in an improvement in the performance data. In these aspects, it has not yet been possible to find any entirely satisfactory solution.

A great influence on the performance data of electronic devices is possessed by emission layers and layers having a hole-transporting function. Novel compounds are also being sought for use in these layers, especially hole-transporting compounds and compounds that can serve as hole-transporting matrix material, especially for phosphorescent emitters, in an emitting layer. For this purpose, especially compounds that have a high glass transition temperature, high stability, and high conductivity for holes are being sought for. A high stability of the compound is a prerequisite for achieving a long lifetime of the electronic device. There is moreover a need to find compounds whose use in electronic devices results in improvement of the performance data of the devices, especially in high efficiency, long lifetime and low operating voltage.

In the prior art, triarylamine compounds in particular, for example spirobifluoreneamines and fluoreneamines, are known as hole transport materials and hole-transporting matrix materials for electronic devices. Also known are heteroaryl-substituted biphenyl amines, such as disclosed in WO2023/025971, WO2019/206292, CN110577510 and CN116478115. Still, there remains room for improvement in respect of the above-mentioned properties.

It has now been found that aromatic amines of the formula (I) below which are characterized in that they comprise a dibenzofuranyl- or dibenzothiophenyl-substituted biphenyl amine moiety, where the dibenzofuranyl and dibenzothiophenyl group are substituted with an aromatic or heteroaromatic group, are of excellent suitability for use in electronic devices. They are especially suitable for use in OLEDs, and even more particularly therein for use as hole transport materials and for use as hole-transporting matrix materials, especially for phosphorescent emitters. The compounds lead to high lifetime, high efficiency and low operating voltage of the devices. Further preferably, the compounds found have a high glass transition temperature, high stability, low sublimation temperature, good solubility, good synthetic accessibility and high conductivity for holes.

The present application is now directed at a compound according to the following formula (I)

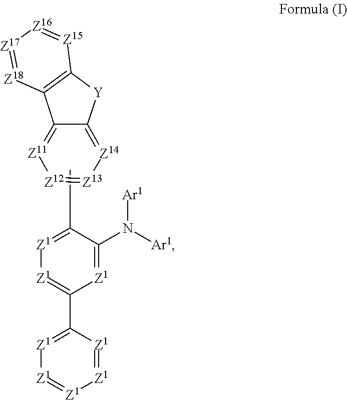

where the following applies to the variables present in the formula:
Z¹ is, identically or differently at each occurrence, selected from CR¹ and N;
Z¹¹, Z¹², Z¹³, Z¹⁴ is each, identically or differently, selected from CR¹, CR¹¹ and N, if the bond drawn into the ring comprising them is not attached to it, and Z¹¹, Z¹², Z¹³, Z¹⁴ is C, if the bond drawn into the ring comprising them is attached to it;
Z¹⁵, Z¹⁶, Z¹⁷, Z¹⁸ is, identically or differently at each occurrence, selected from CR¹, CR¹¹ and N;
Y is selected from O and S;
Ar¹ is selected, identically or differently at each occurrence, from phenyl, biphenyl, terphenyl, and quaterphenyl, where each of phenyl, biphenyl, terphenyl and quaterphenyl is substituted with radicals R²¹, and fluorenyl, which is attached in one of its positions 1 to 4, and which is substituted with radicals R²;
R¹ is selected, identically or differently on each occurrence, from H, D, F, C(═O)R³, CN, Si(R³)₃, N(R³)₂, P(═O)(R³)₂, OR³, S(═O)R³, S(═O)₂R³, straight-chain alkyl or alkoxy groups having 1 to 20 C atoms, branched or cyclic alkyl or alkoxy groups having 3 to 20 C atoms, alkenyl or alkynyl groups having 2 to 20 C atoms, aromatic ring systems having 6 to 40 aromatic ring atoms, and heteroaromatic ring systems having 5 to 40 aromatic ring atoms; where two or more radicals R¹ may be connected to each other to form a ring; where the said alkyl, alkoxy, alkenyl and alkynyl groups and the said aromatic ring systems and heteroaromatic ring systems are substituted by radicals R³, and where one or more CH₂ groups in the said alkyl, alkoxy, alkenyl and alkynyl groups may in each case be replaced by —R³C═CR³—, —C≡C—, Si(R³)₂, C═O, C═NR³, —C(═O)O—, —C(═O)NR³—, NR³, P(═O)(R³), —O—, —S—, SO or SO₂;
R¹¹ is selected, identically or differently on each occurrence, from aromatic ring systems having 6 to 24 aromatic ring atoms and heteroaromatic ring systems having 5 to 24 aromatic ring atoms; where the said said aromatic ring systems and heteroaromatic ring systems are each substituted by radicals R³;
R² is selected, identically or differently on each occurrence, from H, D, F, C(═O)R³, CN, Si(R³)₃, N(R³)₂, P(═O)(R³)₂, OR³, S(═O)R³, S(═O)₂R³, straight-chain alkyl or alkoxy groups having 1 to 20 C atoms, branched or cyclic alkyl or alkoxy groups having 3 to 20 C atoms, alkenyl or alkynyl groups having 2 to 20 C atoms, aromatic ring systems having 6 to 40 aromatic ring atoms, and heteroaromatic ring systems having 5 to 40 aromatic ring atoms; where two or more radicals R² may be connected to each other to form a ring; where the said alkyl, alkoxy, alkenyl and alkynyl groups and the said aromatic ring systems and heteroaromatic ring systems are substituted by radicals R³, and where one or more CH₂ groups in the said alkyl, alkoxy, alkenyl and alkynyl groups may in each case be replaced by —R³C═CR³—, —C≡C—, Si(R³)₂, C═O, C═NR³, —C(═O)O—, —C(═O)NR³—, NR³, P(═O)(R³), —O—, —S—, SO or SO₂;
R²¹ is selected, identically or differently on each occurrence, from H, D, F, C(═O)R³, CN, Si(R³)₃, N(R³)₂, P(═O)(R³)₂, OR³, S(═O)R³, S(═O)₂R³, straight-chain alkyl or alkoxy groups having 1 to 20 C atoms, branched or cyclic alkyl or alkoxy groups having 3 to 20 C atoms, alkenyl or alkynyl groups having 2 to 20 C atoms, where two or more radicals R²¹ may be connected to each other to form a ring; where the said alkyl, alkoxy, alkenyl and alkynyl groups are substituted by radicals R³, and where one or more CH₂ groups in the said alkyl, alkoxy, alkenyl and alkynyl groups may in each case be replaced by —R³C═CR³—, —C≡C—, Si(R³)₂, C═O, C═NR³, —C(═O)O—, —C(═O)NR³—, NR³, P(═O)(R³), —O—, —S—, SO or SO₂;
R³ is selected, identically or differently on each occurrence, from H, D, F, Cl, Br, I, C(═O)R⁴, CN, Si(R⁴)₃, N(R⁴)₂, P(═O)(R⁴)₂, OR⁴, S(═O)R⁴, S(═O)₂R⁴, straight-chain alkyl or alkoxy groups having 1 to 20 C atoms, branched or cyclic alkyl or alkoxy groups having 3 to 20 C atoms, alkenyl or alkynyl groups having 2 to 20 C atoms, aromatic ring systems having 6 to 40 aromatic ring atoms, and heteroaromatic ring systems having 5 to 40 aromatic ring atoms; where two or more radicals R³ may be connected to each other to form a ring; where the said alkyl, alkoxy, alkenyl and alkynyl groups and the said aromatic ring systems and heteroaromatic ring systems are substituted by radicals R⁴, and where one or more CH₂ groups in the said alkyl, alkoxy, alkenyl and alkynyl groups may in each case be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂;
R⁴ is selected, identically or differently on each occurrence, from H, D, F, Cl, Br, I, CN, alkyl groups having 1 to 20 C atoms, aromatic ring systems having 6 to 40 C atoms, and heteroaromatic ring systems having 5 to 40 aromatic ring atoms; where two or more radicals R⁴ may be connected to each other to form a ring; and where the said alkyl groups, aromatic ring systems and heteroaromatic ring systems may be substituted by one or more radicals selected from F and CN;
where the bond drawn into the ring comprising Z¹¹, Z¹², Z¹³ and Z¹⁴ is attached to one of Z¹¹, Z¹², Z¹³, and Z¹⁴, and
where at least one of Z¹¹, Z¹², Z¹³, Z¹⁴, Z¹⁵, Z¹⁶, Z¹⁷ and Z¹⁸ is CR¹¹.

Positions 1 to 4 of fluorenyl are as indicated below:

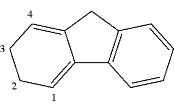 [SMILES: C1=C2Cc3ccccc3C2=CCC1]

The following definitions apply to the chemical groups used as general definitions. They apply insofar as no more specific definitions are given.

An aryl group here is taken to mean either a single aromatic ring, for example benzene, or a condensed aromatic polycycle, for example naphthalene, phenanthrene, or anthracene. A condensed aromatic polycycle in the sense of the present application consists of two or more single aromatic rings which are condensed with one another. An aryl group in the sense of this invention contains 6 to 40 aromatic ring atoms. An aryl group does not contain any heteroatoms as aromatic ring atoms, but only carbon atoms.

A heteroaryl group here is taken to mean either a single heteroaromatic ring, such as pyridine, pyrimidine or thiophene, or a condensed heteroaromatic polycycle, such as quinoline or carbazole. A condensed heteroaromatic polycycle in the sense of the present application consists of two or more single aromatic or heteroaromatic rings, which are condensed with one another, where at least one of the two or more single aromatic or heteroaromatic rings is a heteroaromatic ring. A heteroaryl group in the sense of this invention contains 5 to 40 aromatic ring atoms, at least one of which is a heteroatom. The heteroatoms are preferably selected from N, O and S.

An aryl or heteroaryl group, which may in each case be substituted by the above-mentioned radicals, is taken to mean, in particular, a group derived from benzene, naphthalene, anthracene, phenanthrene, pyrene, dihydropyrene, chrysene, perylene, fluoranthene, benzanthracene, benzophenanthrene, tetracene, pentacene, benzopyrene, furan, benzofuran, isobenzofuran, dibenzofuran, thiophene, benzothio-phene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, benzimidazolo[1,2-a]benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the sense of this invention is a system which does not necessarily contain only aryl groups, but which may additionally contain one or more non-aromatic rings, which are condensed with at least one aryl group. Such non-aromatic rings contain exclusively carbon atoms as ring atoms. Examples of groups embraced by such definition are tetrahydronaphthalene, fluorene, and spirobifluorene. Furthermore, the term aromatic ring system is understood to embrace systems consisting of two or more aromatic ring systems which are connected to each other via single bonds, such as biphenyl, terphenyl, 7-phenyl-2-fluorenyl and quaterphenyl. An aromatic ring system in the sense of this invention contains 6 to 40 C atoms and no heteroatoms as ring atoms of the ring system. An aromatic ring system in the sense of this application does not comprise any heteroaryl groups, as defined above.

A heteroaromatic ring system is defined in analogy to the aromatic ring system above, but with the difference that it must obtain at least one heteroatom as one of the ring atoms. As it is the case for the aromatic ring system, it does not necessarily contain only aryl and heteroaryl groups, but it may additionally contain one or more non-aromatic rings, which are condensed with at least one aryl or heteroaryl group. The non-aromatic rings may contain only carbon atoms as ring atoms, or they may contain additionally one or more heteroatoms, where the heteroatoms are preferably selected from N, O and S. An example for such a heteroaromatic ring system is benzpyranyl. Furthermore, the term heteroaromatic ring system is understood to embrace systems consisting of two or more aromatic or heteroaromatic ring systems, which are connected to each other via single bonds, such as 4,6-diphenyl-2-triazinyl. A heteroaromatic ring system in the sense of this invention contains 5 to 40 ring atoms, which are selected from carbon and heteroatoms, where at least one of the ring atoms is a heteroatom. The heteroatoms are preferably selected from N, O or S.

The terms “heteroaromatic ring system” and “aromatic ring system” according to the definition of the present application differ from each other by the fact that the aromatic ring system cannot comprise any heteroatom as ring atom, whereas the heteroaromatic ring system must comprise at least one heteroatom as ring atom. Such heteroatom may be present as a ring atom of a non-aromatic heterocyclic ring of the system, or as a ring atom of an aromatic heterocyclic ring of the system.

According to the above, any aryl group, as defined above, is embraced by the term “aromatic ring system”, as defined above, and any heteroaryl group, as defined above, is embraced by the term “heteroaromatic ring system”, as defined above.

An aromatic ring system having 6 to 40 aromatic ring atoms or a heteroaromatic ring system having 5 to 40 aromatic ring atoms is in particular a group which is derived from the above-mentioned aryl or heteroaryl groups, or from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, and indenocarbazole, or from any combinations of these groups.

For the purposes of the present invention, a straight-chain alkyl group having 1 to 20 C atoms or a branched or cyclic alkyl group having 3 to 20 C atoms or an alkenyl or alkynyl group having 2 to 20 C atoms, in which, in addition, individual H atoms or CH₂ groups may be substituted by the groups mentioned above under the definition of the radicals, is preferably taken to mean the radicals methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl.

An alkoxy or thioalkyl group having 1 to 20 C atoms is preferably taken to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoro-ethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethyl-hexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The phrase “two or more radicals may be connected to each other to form a ring” shall be understood to include the case that the two radicals are connected by a chemical bond. Additionally, the phrase shall be understood to include the case that one of the two radicals is H, this radical H is removed, and the other of the two radicals forms a ring by being connected to the position, to which this radical H was initially bonded.

Preferably, formula (I) conforms to one of the following formulae (I-A) to (I-D):

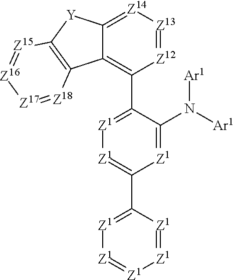

Formula (I-A)

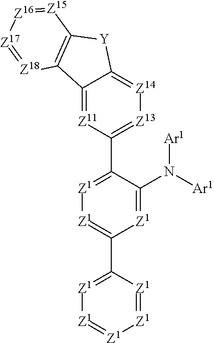

Formula (I-B)

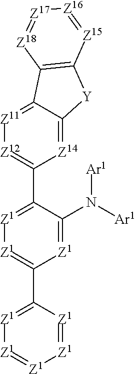

Formula (I-C)

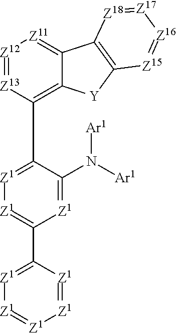

Formula (I-D)

where the variable groups are defined as above, and where Y is preferably 0, and where Z¹, Z¹², Z¹³, Z¹⁴, Z¹⁵, Z¹⁶, Z¹⁷ and Z¹⁸ are selected, identically or differently, from N, CR¹ and CR¹¹, and where Zⁱ is preferably CR¹, and where Z¹¹, Z¹², Z¹³, Z¹⁴, Z¹⁵, Z¹⁶, Z¹⁷ and Z¹⁸ are preferably, identically or differently, selected from CR¹ and CR¹¹, where at least one of Z¹¹, Z¹², Z¹³, Z¹⁴, Z¹⁵, Z¹⁶, Z¹⁷ and Z¹⁸ present in the formula is CR¹¹.

Among the formulae (I-A) to (I-D), formulae (I-B) and (I-C) are preferred.

Preferred embodiments of formulae (I-A) to (I-D) are the following formulae:

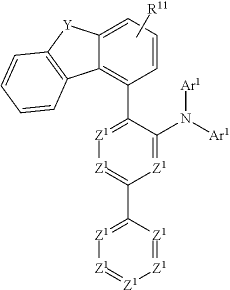

Formula (I-A-a)

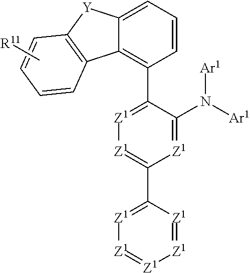

Formula (I-A-b)

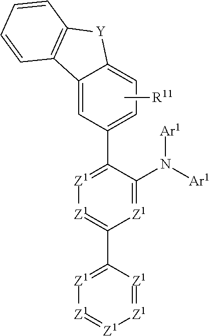

Formula (I-B-a)

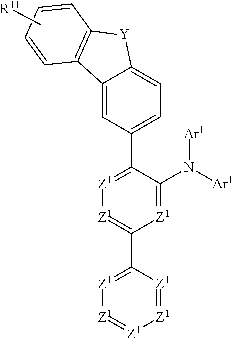

Formula (I-B-b)

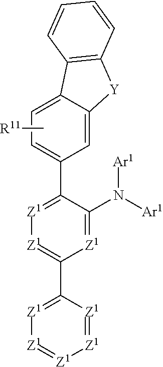

Formula (I-C-a)

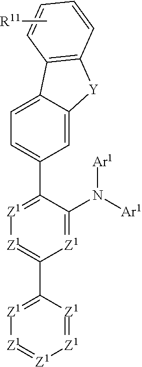

Formula (I-C-b)

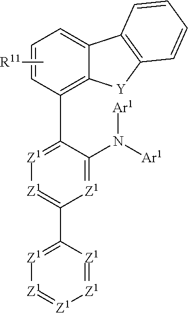

Formula (I-D-a)

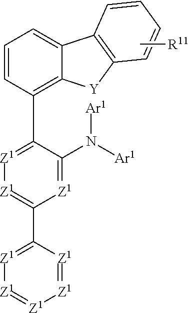

Formula (I-D-b)

where the variable groups are defined as above, and where Y is preferably O, and where Z¹ is preferably CR¹. For clarity, the R¹¹ which has a bond drawn into a ring of the dibenzofuranyl or dibenzothiophenyl group can be bonded to any of the free positions on that ring.

Among the above formulae (I-A-a) to (I-D-b), formulae (I-B-a), (I-B-b), (I-C-a) and (I-C-b) are preferred.

Further, it is preferred that formulae (I-A) to (I-D) conform to the following formulae shown in the table below:

| | basic | | | | | | | | |
|---|---|---|---|---|---|---|---|---|---|
| formula | formula | Z¹¹ | Z¹² | Z¹³ | Z¹⁴ | Z¹⁵ | Z¹⁶ | Z¹⁷ | Z¹⁸ |
| (I-A-1) | (I-A) | C | CR¹¹ | CR¹ | CR¹ | CR¹ | CR¹ | CR¹ | CR¹ |
| (I-A-2) | (I-A) | C | CR¹ | CR¹¹ | CR¹ | CR¹ | CR¹ | CR¹ | CR¹ |
| (I-A-3) | (I-A) | C | CR¹ | CR¹ | CR¹¹ | CR¹ | CR¹ | CR¹ | CR¹ |
| (I-A-4) | (I-A) | C | CR¹ | CR¹ | CR¹ | CR¹¹ | CR¹ | CR¹ | CR¹ |
| (I-A-5) | (I-A) | C | CR¹ | CR¹ | CR¹ | CR¹ | CR¹¹ | CR¹ | CR¹ |
| (I-A-6) | (I-A) | C | CR¹ | CR¹ | CR¹ | CR¹ | CR¹ | CR¹¹ | CR¹ |
| (I-A-7) | (I-A) | C | CR¹ | CR¹ | CR¹ | CR¹ | CR¹ | CR¹ | CR¹¹ |
| (I-B-1) | (I-B) | CR¹¹ | C | CR¹ | CR¹ | CR¹ | CR¹ | CR¹ | CR¹ |
| (I-B-2) | (I-B) | CR¹ | C | CR¹¹ | CR¹ | CR¹ | CR¹ | CR¹ | CR¹ |
| (I-B-3) | (I-B) | CR¹ | C | CR¹ | CR¹¹ | CR¹ | CR¹ | CR¹ | CR¹ |
| (I-B-4) | (I-B) | CR¹ | C | CR¹ | CR¹ | CR¹¹ | CR¹ | CR¹ | CR¹ |
| (I-B-5) | (I-B) | CR¹ | C | CR¹ | CR¹ | CR¹ | CR¹¹ | CR¹ | CR¹ |
| (I-B-6) | (I-B) | CR¹ | C | CR¹ | CR¹ | CR¹ | CR¹ | CR¹¹ | CR¹ |
| (I-B-7) | (I-B) | CR¹ | C | CR¹ | CR¹ | CR¹ | CR¹ | CR¹ | CR¹¹ |
| (I-C-1) | (I-C) | CR¹¹ | CR¹ | C | CR¹ | CR¹ | CR¹ | CR¹ | CR¹ |
| (I-C-2) | (I-C) | CR¹ | CR¹¹ | C | CR¹ | CR¹ | CR¹ | CR¹ | CR¹ |
| (I-C-3) | (I-C) | CR¹ | CR¹ | C | CR¹¹ | CR¹ | CR¹ | CR¹ | CR¹ |
| (I-C-4) | (I-C) | CR¹ | CR¹ | C | CR¹ | CR¹¹ | CR¹ | CR¹ | CR¹ |
| (I-C-5) | (I-C) | CR¹ | CR¹ | C | CR¹ | CR¹ | CR¹¹ | CR¹ | CR¹ |
| (I-C-6) | (I-C) | CR¹ | CR¹ | C | CR¹ | CR¹ | CR¹ | CR¹¹ | CR¹ |
| (I-C-7) | (I-C) | CR¹ | CR¹ | C | CR¹ | CR¹ | CR¹ | CR¹ | CR¹¹ |
| (I-D-1) | (I-D) | CR¹¹ | CR¹ | CR¹ | C | CR¹ | CR¹ | CR¹ | CR¹ |
| (I-D-2) | (I-D) | CR¹ | CR¹¹ | CR¹ | C | CR¹ | CR¹ | CR¹ | CR¹ |
| (I-D-3) | (I-D) | CR¹ | CR¹ | CR¹¹ | C | CR¹ | CR¹ | CR¹ | CR¹ |
| (I-D-4) | (I-D) | CR¹ | CR¹ | CR¹ | C | CR¹¹ | CR¹ | CR¹ | CR¹ |
| (I-D-5) | (I-D) | CR¹ | CR¹ | CR¹ | C | CR¹ | CR¹¹ | CR¹ | CR¹ |
| (I-D-6) | (I-D) | CR¹ | CR¹ | CR¹ | C | CR¹ | CR¹ | CR¹¹ | CR¹ |
| (I-D-7) | (I-D) | CR¹ | CR¹ | CR¹ | C | CR¹ | CR¹ | CR¹ | CR¹¹ |

For the above formulae in the table, it is preferred that Y is O. Further, it is preferred that R¹ is selected, identically or differently on each occurrence, from H and D; and most preferably, R¹ is H.

Among the above formulae (I-A-1) to (I-D-7), formulae (I-A-3), (I-B-3), (I-C-1), (I-C-4), (I-C-7), (I-D-1), and (I-D-4) are preferred.

Preferred embodiments of the compound of formula (I) conform to the following formulae, where in formula (I), the moiety A marked with a box

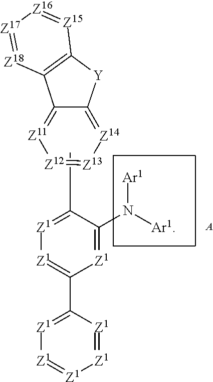

is selected from the following groups as shown in the column on the right:

| A group | chemical formula |
|---|---|
| (A-1) | 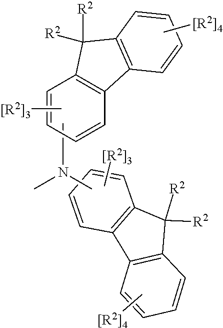 |
| (A-2) | 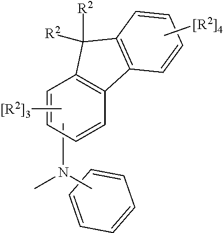 |
| (A-3) | 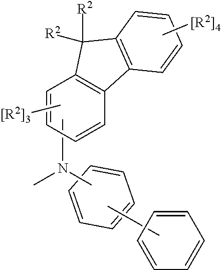 |
| (A-4) | 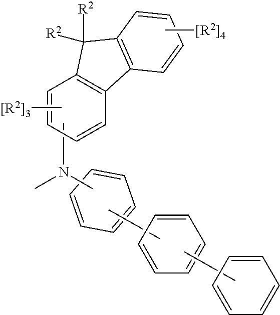 |
| (A-5) | 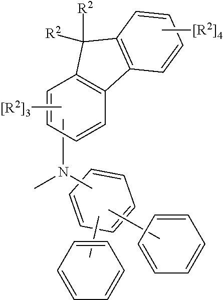 |
| (A-6) | 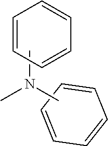 |
| (A-7) | 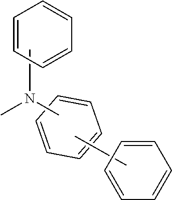 |
| (A-8) | 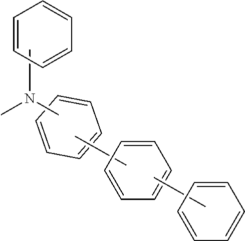 |
| (A-9) | 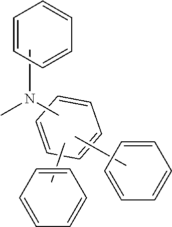 |
| (A-10) | 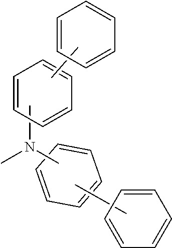 |
| (A-11) | 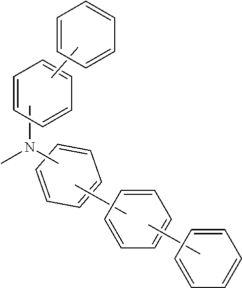 |
| (A-12) | 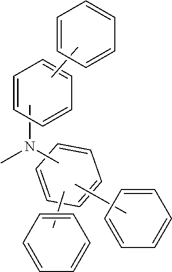 |
| (A-13) | 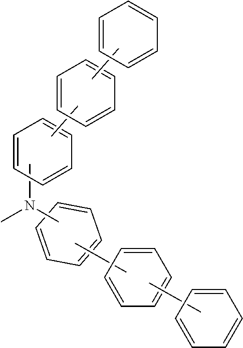 |
| (A-14) | 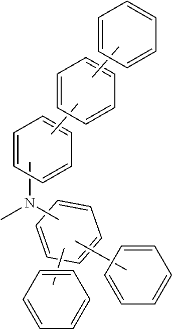 |
| (A-15) | 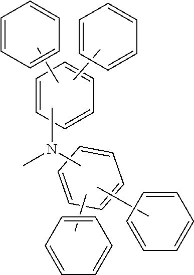 |

where each of the free positions on the aromatic rings of the phenyl, biphenyl and terphenyl groups is substituted with a radical R²¹, which is in this case preferably identically or differently H or D, most preferably H.

Among the above preferred embodiments of A, groups (A-1), (A-3), (A-4), (A-10) and (A-11) are even more preferred. Most preferred embodiments of A are group (A-1) and (A-3).

Preferred embodiments of formula (I) thus conform to the following formulae:

| Formula | Basic formula | Formula of group A |
|---|---|---|
| (I-1) | (I) | (A-1) |
| (I-2) | (I) | (A-2) |
| (I-3) | (I) | (A-3) |
| (I-4) | (I) | (A-4) |
| (I-5) | (I) | (A-5) |
| (I-6) | (I) | (A-6) |
| (I-7) | (I) | (A-7) |
| (I-8) | (I) | (A-8) |
| (I-9) | (I) | (A-9) |
| (I-10) | (I) | (A-10) |
| (I-11) | (I) | (A-11) |
| (I-12) | (I) | (A-12) |
| (I-13) | (I) | (A-13) |
| (I-14) | (I) | (A-14) |
| (I-15) | (I) | (A-15) |

where each of the free positions on the aromatic rings of the phenyl, biphenyl and terphenyl groups is substituted with a radical R²¹.

Further preferred embodiments of formula (I) conform to the following formulae:

| Formula | Basic formula | Formula of group A |
|---|---|---|
| (I-A-a) | (I-A) | (A-1) |
| (I-A-b) | (I-A) | (A-2) |
| (I-A-c) | (I-A) | (A-3) |
| (I-A-d) | (I-A) | (A-4) |
| (I-A-e) | (I-A) | (A-5) |
| (I-A-f) | (I-A) | (A-6) |
| (I-A-g) | (I-A) | (A-7) |
| (I-A-h) | (I-A) | (A-8) |
| (I-A-i) | (I-A) | (A-9) |
| (I-A-j) | (I-A) | (A-10) |
| (I-A-k) | (I-A) | (A-11) |
| (I-A-l) | (I-A) | (A-12) |
| (I-A-m) | (I-A) | (A-13) |
| (I-A-n) | (I-A) | (A-14) |
| (I-A-o) | (I-A) | (A-15) |
| (I-B-a) | (I-B) | (A-1) |
| (I-B-b) | (I-B) | (A-2) |
| (I-B-c) | (I-B) | (A-3) |
| (I-B-d) | (I-B) | (A-4) |
| (I-B-e) | (I-B) | (A-5) |
| (I-B-f) | (I-B) | (A-6) |
| (I-B-g) | (I-B) | (A-7) |
| (I-B-h) | (I-B) | (A-8) |
| (I-B-i) | (I-B) | (A-9) |
| (I-B-j) | (I-B) | (A-10) |
| (I-B-k) | (I-B) | (A-11) |
| (I-B-l) | (I-B) | (A-12) |
| (I-B-m) | (I-B) | (A-13) |
| (I-B-n) | (I-B) | (A-14) |
| (I-B-o) | (I-B) | (A-15) |
| (I-C-a) | (I-C) | (A-1) |
| (I-C-b) | (I-C) | (A-2) |
| (I-C-c) | (I-C) | (A-3) |
| (I-C-d) | (I-C) | (A-4) |
| (I-C-e) | (I-C) | (A-5) |
| (I-C-f) | (I-C) | (A-6) |
| (I-C-g) | (I-C) | (A-7) |
| (I-C-h) | (I-C) | (A-8) |
| (I-C-i) | (I-C) | (A-9) |
| (I-C-j) | (I-C) | (A-10) |
| (I-C-k) | (I-C) | (A-11) |
| (I-C-l) | (I-C) | (A-12) |
| (I-C-m) | (I-C) | (A-13) |
| (I-C-n) | (I-C) | (A-14) |
| (I-C-o) | (I-C) | (A-15) |
| (I-D-a) | (I-D) | (A-1) |
| (I-D-b) | (I-D) | (A-2) |
| (I-D-c) | (I-D) | (A-3) |
| (I-D-d) | (I-D) | (A-4) |
| (I-D-e) | (I-D) | (A-5) |
| (I-D-f) | (I-D) | (A-6) |
| (I-D-g) | (I-D) | (A-7) |
| (I-D-h) | (I-D) | (A-8) |
| (I-D-i) | (I-D) | (A-9) |
| (I-D-j) | (I-D) | (A-10) |
| (I-D-k) | (I-D) | (A-11) |
| (I-D-l) | (I-D) | (A-12) |
| (I-D-m) | (I-D) | (A-13) |
| (I-D-n) | (I-D) | (A-14) |
| (I-D-o) | (I-D) | (A-15) |

where the variable definitions as above apply, and where the preferences mentioned above for formulae (I-A) to (I-D) apply, and where each of the free positions on the aromatic rings of the phenyl, biphenyl and terphenyl groups is substituted with a radical R²¹, which is in this case preferably identically or differently H or D, most preferably H.

Further preferred embodiments of formula (I) conform to the following formulae:

| Formula | Basic formula | Formula of group A |
|---|---|---|
| (I-A-1-a) | (I-A-1) | (A-1) |
| (I-A-1-b) | (I-A-1) | (A-2) |
| (I-A-1-c) | (I-A-1) | (A-3) |
| (I-A-1-d) | (I-A-1) | (A-4) |
| (I-A-1-e) | (I-A-1) | (A-5) |
| (I-A-1-f) | (I-A-1) | (A-6) |
| (I-A-1-g) | (I-A-1) | (A-7) |
| (I-A-1-h) | (I-A-1) | (A-8) |
| (I-A-1-i) | (I-A-1) | (A-9) |
| (I-A-1-j) | (I-A-1) | (A-10) |
| (I-A-1-k) | (I-A-1) | (A-11) |
| (I-A-1-l) | (I-A-1) | (A-12) |
| (I-A-1-m) | (I-A-1) | (A-13) |
| (I-A-1-n) | (I-A-1) | (A-14) |
| (I-A-1-o) | (I-A-1) | (A-15) |
| (I-A-2-a) | (I-A-2) | (A-1) |
| (I-A-2-b) | (I-A-2) | (A-2) |
| (I-A-2-c) | (I-A-2) | (A-3) |
| (I-A-2-d) | (I-A-2) | (A-4) |
| (I-A-2-e) | (I-A-2) | (A-5) |
| (I-A-2-f) | (I-A-2) | (A-6) |
| (I-A-2-g) | (I-A-2) | (A-7) |
| (I-A-2-h) | (I-A-2) | (A-8) |
| (I-A-2-i) | (I-A-2) | (A-9) |
| (I-A-2-j) | (I-A-2) | (A-10) |
| (I-A-2-k) | (I-A-2) | (A-11) |
| (I-A-2-l) | (I-A-2) | (A-12) |
| (I-A-2-m) | (I-A-2) | (A-13) |
| (I-A-2-n) | (I-A-2) | (A-14) |
| (I-A-2-o) | (I-A-2) | (A-15) |
| (I-A-3-a) | (I-A-3) | (A-1) |
| (I-A-3-b) | (I-A-3) | (A-2) |
| (I-A-3-c) | (I-A-3) | (A-3) |
| (I-A-3-d) | (I-A-3) | (A-4) |
| (I-A-3-e) | (I-A-3) | (A-5) |
| (I-A-3-f) | (I-A-3) | (A-6) |
| (I-A-3-g) | (I-A-3) | (A-7) |
| (I-A-3-h) | (I-A-3) | (A-8) |
| (I-A-3-i) | (I-A-3) | (A-9) |
| (I-A-3-j) | (I-A-3) | (A-10) |
| (I-A-3-k) | (I-A-3) | (A-11) |
| (I-A-3-l) | (I-A-3) | (A-12) |
| (I-A-3-m) | (I-A-3) | (A-13) |
| (I-A-3-n) | (I-A-3) | (A-14) |
| (I-A-3-o) | (I-A-3) | (A-15) |
| (I-A-4-a) | (I-A-4) | (A-1) |
| (I-A-4-b) | (I-A-4) | (A-2) |
| (I-A-4-c) | (I-A-4) | (A-3) |
| (I-A-4-d) | (I-A-4) | (A-4) |
| (I-A-4-e) | (I-A-4) | (A-5) |
| (I-A-4-f) | (I-A-4) | (A-6) |
| (I-A-4-g) | (I-A-4) | (A-7) |
| (I-A-4-h) | (I-A-4) | (A-8) |
| (I-A-4-i) | (I-A-4) | (A-9) |
| (I-A-4-j) | (I-A-4) | (A-10) |
| (I-A-4-k) | (I-A-4) | (A-11) |
| (I-A-4-l) | (I-A-4) | (A-12) |
| (I-A-4-m) | (I-A-4) | (A-13) |
| (I-A-4-n) | (I-A-4) | (A-14) |
| (I-A-4-o) | (I-A-4) | (A-15) |
| (I-A-5-a) | (I-A-5) | (A-1) |
| (I-A-5-b) | (I-A-5) | (A-2) |
| (I-A-5-c) | (I-A-5) | (A-3) |
| (I-A-5-d) | (I-A-5) | (A-4) |
| (I-A-5-e) | (I-A-5) | (A-5) |
| (I-A-5-f) | (I-A-5) | (A-6) |
| (I-A-5-g) | (I-A-5) | (A-7) |
| (I-A-5-h) | (I-A-5) | (A-8) |
| (I-A-5-i) | (I-A-5) | (A-9) |
| (I-A-5-j) | (I-A-5) | (A-10) |
| (I-A-5-k) | (I-A-5) | (A-11) |
| (I-A-5-l) | (I-A-5) | (A-12) |
| (I-A-5-m) | (I-A-5) | (A-13) |
| (I-A-5-n) | (I-A-5) | (A-14) |
| (I-A-5-o) | (I-A-5) | (A-15) |
| (I-A-6-a) | (I-A-6) | (A-1) |
| (I-A-6-b) | (I-A-6) | (A-2) |
| (I-A-6-c) | (I-A-6) | (A-3) |
| (I-A-6-d) | (I-A-6) | (A-4) |
| (I-A-6-e) | (I-A-6) | (A-5) |
| (I-A-6-f) | (I-A-6) | (A-6) |
| (I-A-6-g) | (I-A-6) | (A-7) |
| (I-A-6-h) | (I-A-6) | (A-8) |
| (I-A-6-i) | (I-A-6) | (A-9) |
| (I-A-6-j) | (I-A-6) | (A-10) |
| (I-A-6-k) | (I-A-6) | (A-11) |
| (I-A-6-l) | (I-A-6) | (A-12) |
| (I-A-6-m) | (I-A-6) | (A-13) |
| (I-A-6-n) | (I-A-6) | (A-14) |
| (I-A-6-o) | (I-A-6) | (A-15) |
| (I-A-7-a) | (I-A-7) | (A-1) |
| (I-A-7-b) | (I-A-7) | (A-2) |
| (I-A-7-c) | (I-A-7) | (A-3) |
| (I-A-7-d) | (I-A-7) | (A-4) |
| (I-A-7-e) | (I-A-7) | (A-5) |
| (I-A-7-f) | (I-A-7) | (A-6) |
| (I-A-7-g) | (I-A-7) | (A-7) |
| (I-A-7-h) | (I-A-7) | (A-8) |
| (I-A-7-i) | (I-A-7) | (A-9) |
| (I-A-7-j) | (I-A-7) | (A-10) |
| (I-A-7-k) | (I-A-7) | (A-11) |
| (I-A-7-l) | (I-A-7) | (A-12) |
| (I-A-7-m) | (I-A-7) | (A-13) |
| (I-A-7-n) | (I-A-7) | (A-14) |
| (I-A-7-o) | (I-A-7) | (A-15) |
| (I-B-1-a) | (I-B-1) | (A-1) |
| (I-B-1-b) | (I-B-1) | (A-2) |
| (I-B-1-c) | (I-B-1) | (A-3) |
| (I-B-1-d) | (I-B-1) | (A-4) |
| (I-B-1-e) | (I-B-1) | (A-5) |
| (I-B-1-f) | (I-B-1) | (A-6) |
| (I-B-1-g) | (I-B-1) | (A-7) |
| (I-B-1-h) | (I-B-1) | (A-8) |
| (I-B-1-i) | (I-B-1) | (A-9) |
| (I-B-1-j) | (I-B-1) | (A-10) |
| (I-B-1-k) | (I-B-1) | (A-11) |
| (I-B-1-l) | (I-B-1) | (A-12) |
| (I-B-1-m) | (I-B-1) | (A-13) |
| (I-B-1-n) | (I-B-1) | (A-14) |
| (I-B-1-o) | (I-B-1) | (A-15) |
| (I-B-2-a) | (I-B-2) | (A-1) |
| (I-B-2-b) | (I-B-2) | (A-2) |
| (I-B-2-c) | (I-B-2) | (A-3) |
| (I-B-2-d) | (I-B-2) | (A-4) |
| (I-B-2-e) | (I-B-2) | (A-5) |
| (I-B-2-f) | (I-B-2) | (A-6) |
| (I-B-2-g) | (I-B-2) | (A-7) |
| (I-B-2-h) | (I-B-2) | (A-8) |
| (I-B-2-i) | (I-B-2) | (A-9) |
| (I-B-2-j) | (I-B-2) | (A-10) |
| (I-B-2-k) | (I-B-2) | (A-11) |
| (I-B-2-l) | (I-B-2) | (A-12) |
| (I-B-2-m) | (I-B-2) | (A-13) |
| (I-B-2-n) | (I-B-2) | (A-14) |
| (I-B-2-o) | (I-B-2) | (A-15) |
| (I-B-3-a) | (I-B-3) | (A-1) |
| (I-B-3-b) | (I-B-3) | (A-2) |
| (I-B-3-c) | (I-B-3) | (A-3) |
| (I-B-3-d) | (I-B-3) | (A-4) |
| (I-B-3-e) | (I-B-3) | (A-5) |
| (I-B-3-f) | (I-B-3) | (A-6) |
| (I-B-3-g) | (I-B-3) | (A-7) |
| (I-B-3-h) | (I-B-3) | (A-8) |
| (I-B-3-i) | (I-B-3) | (A-9) |
| (I-B-3-j) | (I-B-3) | (A-10) |
| (I-B-3-k) | (I-B-3) | (A-11) |
| (I-B-3-l) | (I-B-3) | (A-12) |
| (I-B-3-m) | (I-B-3) | (A-13) |
| (I-B-3-n) | (I-B-3) | (A-14) |
| (I-B-3-o) | (I-B-3) | (A-15) |
| (I-B-4-a) | (I-B-4) | (A-1) |
| (I-B-4-b) | (I-B-4) | (A-2) |
| (I-B-4-c) | (I-B-4) | (A-3) |
| (I-B-4-d) | (I-B-4) | (A-4) |
| (I-B-4-e) | (I-B-4) | (A-5) |
| (I-B-4-f) | (I-B-4) | (A-6) |
| (I-B-4-g) | (I-B-4) | (A-7) |
| (I-B-4-h) | (I-B-4) | (A-8) |
| (I-B-4-i) | (I-B-4) | (A-9) |
| (I-B-4-j) | (I-B-4) | (A-10) |
| (I-B-4-k) | (I-B-4) | (A-11) |
| (I-B-4-l) | (I-B-4) | (A-12) |
| (I-B-4-m) | (I-B-4) | (A-13) |
| (I-B-4-n) | (I-B-4) | (A-14) |
| (I-B-4-o) | (I-B-4) | (A-15) |
| (I-B-5-a) | (I-B-5) | (A-1) |
| (I-B-5-b) | (I-B-5) | (A-2) |
| (I-B-5-c) | (I-B-5) | (A-3) |
| (I-B-5-d) | (I-B-5) | (A-4) |
| (I-B-5-e) | (I-B-5) | (A-5) |
| (I-B-5-f) | (I-B-5) | (A-6) |
| (I-B-5-g) | (I-B-5) | (A-7) |
| (I-B-5-h) | (I-B-5) | (A-8) |
| (I-B-5-i) | (I-B-5) | (A-9) |
| (I-B-5-j) | (I-B-5) | (A-10) |
| (I-B-5-k) | (I-B-5) | (A-11) |
| (I-B-5-l) | (I-B-5) | (A-12) |
| (I-B-5-m) | (I-B-5) | (A-13) |
| (I-B-5-n) | (I-B-5) | (A-14) |
| (I-B-5-o) | (I-B-5) | (A-15) |
| (I-B-6-a) | (I-B-6) | (A-1) |
| (I-B-6-b) | (I-B-6) | (A-2) |
| (I-B-6-c) | (I-B-6) | (A-3) |
| (I-B-6-d) | (I-B-6) | (A-4) |
| (I-B-6-e) | (I-B-6) | (A-5) |
| (I-B-6-f) | (I-B-6) | (A-6) |
| (I-B-6-g) | (I-B-6) | (A-7) |
| (I-B-6-h) | (I-B-6) | (A-8) |
| (I-B-6-i) | (I-B-6) | (A-9) |
| (I-B-6-j) | (I-B-6) | (A-10) |
| (I-B-6-k) | (I-B-6) | (A-11) |
| (I-B-6-l) | (I-B-6) | (A-12) |
| (I-B-6-m) | (I-B-6) | (A-13) |
| (I-B-6-n) | (I-B-6) | (A-14) |
| (I-B-6-o) | (I-B-6) | (A-15) |
| (I-B-7-a) | (I-B-7) | (A-1) |
| (I-B-7-b) | (I-B-7) | (A-2) |
| (I-B-7-c) | (I-B-7) | (A-3) |
| (I-B-7-d) | (I-B-7) | (A-4) |
| (I-B-7-e) | (I-B-7) | (A-5) |
| (I-B-7-f) | (I-B-7) | (A-6) |
| (I-B-7-g) | (I-B-7) | (A-7) |
| (I-B-7-h) | (I-B-7) | (A-8) |
| (I-B-7-i) | (I-B-7) | (A-9) |
| (I-B-7-j) | (I-B-7) | (A-10) |
| (I-B-7-k) | (I-B-7) | (A-11) |
| (I-B-7-l) | (I-B-7) | (A-12) |
| (I-B-7-m) | (I-B-7) | (A-13) |
| (I-B-7-n) | (I-B-7) | (A-14) |
| (I-B-7-o) | (I-B-7) | (A-15) |
| (I-C-1-a) | (I-C-1) | (A-1) |
| (I-C-1-b) | (I-C-1) | (A-2) |
| (I-C-1-c) | (I-C-1) | (A-3) |
| (I-C-1-d) | (I-C-1) | (A-4) |
| (I-C-1-e) | (I-C-1) | (A-5) |
| (I-C-1-f) | (I-C-1) | (A-6) |
| (I-C-1-g) | (I-C-1) | (A-7) |
| (I-C-1-h) | (I-C-1) | (A-8) |
| (I-C-1-i) | (I-C-1) | (A-9) |
| (I-C-1-j) | (I-C-1) | (A-10) |
| (I-C-1-k) | (I-C-1) | (A-11) |
| (I-C-1-l) | (I-C-1) | (A-12) |
| (I-C-1-m) | (I-C-1) | (A-13) |
| (I-C-1-n) | (I-C-1) | (A-14) |
| (I-C-1-o) | (I-C-1) | (A-15) |
| (I-C-2-a) | (I-C-2) | (A-1) |
| (I-C-2-b) | (I-C-2) | (A-2) |
| (I-C-2-c) | (I-C-2) | (A-3) |
| (I-C-2-d) | (I-C-2) | (A-4) |
| (I-C-2-e) | (I-C-2) | (A-5) |
| (I-C-2-f) | (I-C-2) | (A-6) |
| (I-C-2-g) | (I-C-2) | (A-7) |
| (I-C-2-h) | (I-C-2) | (A-8) |
| (I-C-2-i) | (I-C-2) | (A-9) |
| (I-C-2-j) | (I-C-2) | (A-10) |
| (I-C-2-k) | (I-C-2) | (A-11) |
| (I-C-2-l) | (I-C-2) | (A-12) |
| (I-C-2-m) | (I-C-2) | (A-13) |
| (I-C-2-n) | (I-C-2) | (A-14) |
| (I-C-2-o) | (I-C-2) | (A-15) |
| (I-C-3-a) | (I-C-3) | (A-1) |
| (I-C-3-b) | (I-C-3) | (A-2) |
| (I-C-3-c) | (I-C-3) | (A-3) |
| (I-C-3-d) | (I-C-3) | (A-4) |
| (I-C-3-e) | (I-C-3) | (A-5) |
| (I-C-3-f) | (I-C-3) | (A-6) |
| (I-C-3-g) | (I-C-3) | (A-7) |
| (I-C-3-h) | (I-C-3) | (A-8) |
| (I-C-3-i) | (I-C-3) | (A-9) |
| (I-C-3-j) | (I-C-3) | (A-10) |
| (I-C-3-k) | (I-C-3) | (A-11) |
| (I-C-3-l) | (I-C-3) | (A-12) |
| (I-C-3-m) | (I-C-3) | (A-13) |
| (I-C-3-n) | (I-C-3) | (A-14) |
| (I-C-3-o) | (I-C-3) | (A-15) |
| (I-C-4-a) | (I-C-4) | (A-1) |
| (I-C-4-b) | (I-C-4) | (A-2) |
| (I-C-4-c) | (I-C-4) | (A-3) |
| (I-C-4-d) | (I-C-4) | (A-4) |
| (I-C-4-e) | (I-C-4) | (A-5) |
| (I-C-4-f) | (I-C-4) | (A-6) |
| (I-C-4-g) | (I-C-4) | (A-7) |
| (I-C-4-h) | (I-C-4) | (A-8) |
| (I-C-4-i) | (I-C-4) | (A-9) |
| (I-C-4-j) | (I-C-4) | (A-10) |
| (I-C-4-k) | (I-C-4) | (A-11) |
| (I-C-4-l) | (I-C-4) | (A-12) |
| (I-C-4-m) | (I-C-4) | (A-13) |
| (I-C-4-n) | (I-C-4) | (A-14) |
| (I-C-4-o) | (I-C-4) | (A-15) |
| (I-C-5-a) | (I-C-5) | (A-1) |
| (I-C-5-b) | (I-C-5) | (A-2) |
| (I-C-5-c) | (I-C-5) | (A-3) |
| (I-C-5-d) | (I-C-5) | (A-4) |
| (I-C-5-e) | (I-C-5) | (A-5) |
| (I-C-5-f) | (I-C-5) | (A-6) |
| (I-C-5-g) | (I-C-5) | (A-7) |
| (I-C-5-h) | (I-C-5) | (A-8) |
| (I-C-5-i) | (I-C-5) | (A-9) |
| (I-C-5-j) | (I-C-5) | (A-10) |
| (I-C-5-k) | (I-C-5) | (A-11) |
| (I-C-5-l) | (I-C-5) | (A-12) |
| (I-C-5-m) | (I-C-5) | (A-13) |
| (I-C-5-n) | (I-C-5) | (A-14) |
| (I-C-5-o) | (I-C-5) | (A-15) |
| (I-C-6-a) | (I-C-6) | (A-1) |
| (I-C-6-b) | (I-C-6) | (A-2) |
| (I-C-6-c) | (I-C-6) | (A-3) |
| (I-C-6-d) | (I-C-6) | (A-4) |
| (I-C-6-e) | (I-C-6) | (A-5) |
| (I-C-6-f) | (I-C-6) | (A-6) |
| (I-C-6-g) | (I-C-6) | (A-7) |
| (I-C-6-h) | (I-C-6) | (A-8) |
| (I-C-6-i) | (I-C-6) | (A-9) |
| (I-C-6-j) | (I-C-6) | (A-10) |
| (I-C-6-k) | (I-C-6) | (A-11) |
| (I-C-6-l) | (I-C-6) | (A-12) |
| (I-C-6-m) | (I-C-6) | (A-13) |
| (I-C-6-n) | (I-C-6) | (A-14) |
| (I-C-6-o) | (I-C-6) | (A-15) |
| (I-C-7-a) | (I-C-7) | (A-1) |
| (I-C-7-b) | (I-C-7) | (A-2) |
| (I-C-7-c) | (I-C-7) | (A-3) |
| (I-C-7-d) | (I-C-7) | (A-4) |
| (I-C-7-e) | (I-C-7) | (A-5) |
| (I-C-7-f) | (I-C-7) | (A-6) |
| (I-C-7-g) | (I-C-7) | (A-7) |
| (I-C-7-h) | (I-C-7) | (A-8) |
| (I-C-7-i) | (I-C-7) | (A-9) |
| (I-C-7-j) | (I-C-7) | (A-10) |
| (I-C-7-k) | (I-C-7) | (A-11) |
| (I-C-7-l) | (I-C-7) | (A-12) |
| (I-C-7-m) | (I-C-7) | (A-13) |
| (I-C-7-n) | (I-C-7) | (A-14) |
| (I-C-7-o) | (I-C-7) | (A-15) |
| (I-D-1-a) | (I-D-1) | (A-1) |
| (I-D-1-b) | (I-D-1) | (A-2) |
| (I-D-1-c) | (I-D-1) | (A-3) |
| (I-D-1-d) | (I-D-1) | (A-4) |
| (I-D-1-e) | (I-D-1) | (A-5) |
| (I-D-1-f) | (I-D-1) | (A-6) |
| (I-D-1-g) | (I-D-1) | (A-7) |
| (I-D-1-h) | (I-D-1) | (A-8) |
| (I-D-1-i) | (I-D-1) | (A-9) |
| (I-D-1-j) | (I-D-1) | (A-10) |
| (I-D-1-k) | (I-D-1) | (A-11) |
| (I-D-1-l) | (I-D-1) | (A-12) |
| (I-D-1-m) | (I-D-1) | (A-13) |
| (I-D-1-n) | (I-D-1) | (A-14) |
| (I-D-1-o) | (I-D-1) | (A-15) |
| (I-D-2-a) | (I-D-2) | (A-1) |
| (I-D-2-b) | (I-D-2) | (A-2) |
| (I-D-2-c) | (I-D-2) | (A-3) |
| (I-D-2-d) | (I-D-2) | (A-4) |
| (I-D-2-e) | (I-D-2) | (A-5) |
| (I-D-2-f) | (I-D-2) | (A-6) |
| (I-D-2-g) | (I-D-2) | (A-7) |
| (I-D-2-h) | (I-D-2) | (A-8) |
| (I-D-2-i) | (I-D-2) | (A-9) |
| (I-D-2-j) | (I-D-2) | (A-10) |
| (I-D-2-k) | (I-D-2) | (A-11) |
| (I-D-2-l) | (I-D-2) | (A-12) |
| (I-D-2-m) | (I-D-2) | (A-13) |
| (I-D-2-n) | (I-D-2) | (A-14) |
| (I-D-2-o) | (I-D-2) | (A-15) |
| (I-D-3-a) | (I-D-3) | (A-1) |
| (I-D-3-b) | (I-D-3) | (A-2) |
| (I-D-3-c) | (I-D-3) | (A-3) |
| (I-D-3-d) | (I-D-3) | (A-4) |
| (I-D-3-e) | (I-D-3) | (A-5) |
| (I-D-3-f) | (I-D-3) | (A-6) |
| (I-D-3-g) | (I-D-3) | (A-7) |
| (I-D-3-h) | (I-D-3) | (A-8) |
| (I-D-3-i) | (I-D-3) | (A-9) |
| (I-D-3-j) | (I-D-3) | (A-10) |
| (I-D-3-k) | (I-D-3) | (A-11) |
| (I-D-3-l) | (I-D-3) | (A-12) |
| (I-D-3-m) | (I-D-3) | (A-13) |
| (I-D-3-n) | (I-D-3) | (A-14) |
| (I-D-3-o) | (I-D-3) | (A-15) |
| (I-D-4-a) | (I-D-4) | (A-1) |
| (I-D-4-b) | (I-D-4) | (A-2) |
| (I-D-4-c) | (I-D-4) | (A-3) |
| (I-D-4-d) | (I-D-4) | (A-4) |
| (I-D-4-e) | (I-D-4) | (A-5) |
| (I-D-4-f) | (I-D-4) | (A-6) |
| (I-D-4-g) | (I-D-4) | (A-7) |
| (I-D-4-h) | (I-D-4) | (A-8) |
| (I-D-4-i) | (I-D-4) | (A-9) |
| (I-D-4-j) | (I-D-4) | (A-10) |
| (I-D-4-k) | (I-D-4) | (A-11) |
| (I-D-4-l) | (I-D-4) | (A-12) |
| (I-D-4-m) | (I-D-4) | (A-13) |
| (I-D-4-n) | (I-D-4) | (A-14) |
| (I-D-4-o) | (I-D-4) | (A-15) |
| (I-D-5-a) | (I-D-5) | (A-1) |
| (I-D-5-b) | (I-D-5) | (A-2) |
| (I-D-5-c) | (I-D-5) | (A-3) |
| (I-D-5-d) | (I-D-5) | (A-4) |
| (I-D-5-e) | (I-D-5) | (A-5) |
| (I-D-5-f) | (I-D-5) | (A-6) |
| (I-D-5-g) | (I-D-5) | (A-7) |
| (I-D-5-h) | (I-D-5) | (A-8) |
| (I-D-5-i) | (I-D-5) | (A-9) |
| (I-D-5-j) | (I-D-5) | (A-10) |
| (I-D-5-k) | (I-D-5) | (A-11) |
| (I-D-5-l) | (I-D-5) | (A-12) |
| (I-D-5-m) | (I-D-5) | (A-13) |
| (I-D-5-n) | (I-D-5) | (A-14) |
| (I-D-5-o) | (I-D-5) | (A-15) |
| (I-D-6-a) | (I-D-6) | (A-1) |
| (I-D-6-b) | (I-D-6) | (A-2) |
| (I-D-6-c) | (I-D-6) | (A-3) |
| (I-D-6-d) | (I-D-6) | (A-4) |
| (I-D-6-e) | (I-D-6) | (A-5) |
| (I-D-6-f) | (I-D-6) | (A-6) |
| (I-D-6-g) | (I-D-6) | (A-7) |
| (I-D-6-h) | (I-D-6) | (A-8) |
| (I-D-6-i) | (I-D-6) | (A-9) |
| (I-D-6-j) | (I-D-6) | (A-10) |
| (I-D-6-k) | (I-D-6) | (A-11) |
| (I-D-6-l) | (I-D-6) | (A-12) |
| (I-D-6-m) | (I-D-6) | (A-13) |
| (I-D-6-n) | (I-D-6) | (A-14) |
| (I-D-6-o) | (I-D-6) | (A-15) |
| (I-D-7-a) | (I-D-7) | (A-1) |
| (I-D-7-b) | (I-D-7) | (A-2) |
| (I-D-7-c) | (I-D-7) | (A-3) |
| (I-D-7-d) | (I-D-7) | (A-4) |
| (I-D-7-e) | (I-D-7) | (A-5) |
| (I-D-7-f) | (I-D-7) | (A-6) |
| (I-D-7-g) | (I-D-7) | (A-7) |
| (I-D-7-h) | (I-D-7) | (A-8) |
| (I-D-7-i) | (I-D-7) | (A-9) |
| (I-D-7-j) | (I-D-7) | (A-10) |
| (I-D-7-k) | (I-D-7) | (A-11) |
| (I-D-7-l) | (I-D-7) | (A-12) |
| (I-D-7-m) | (I-D-7) | (A-13) |
| (I-D-7-n) | (I-D-7) | (A-14) |
| (I-D-7-o) | (I-D-7) | (A-15) |

where the variable definitions as above apply, and where the preferences mentioned above for formulae (I-A-1) to (I-D-7) apply, and where each of the free positions on the aromatic rings of the phenyl, biphenyl and terphenyl groups is substituted with a radical R²¹, which is in this case preferably identically or differently H or D, most preferably H.

According to a preferred embodiment, Z¹ is CR¹. According to a further preferred embodiment, not more than 3 Z¹ in one ring are N, more preferably not more than 2, even more preferably not more than 1. Most preferably, no Z¹ is N, and all Z¹ in a ring is CR¹.

According to a preferred embodiment, Z¹¹, Z¹², Z¹³, Z¹⁴ is each, identically or differently, selected from CR¹ and CR¹¹, if the bond drawn into the ring comprising them is not attached to it, while Z¹¹, Z¹², Z¹³, Z¹⁴ is C, if the bond drawn into the ring comprising them is attached to it.

According to a preferred embodiment, Z¹⁵, Z¹⁶, Z¹⁷, Z¹⁸ is, identically or differently at each occurrence, selected from CR¹ and CR¹¹.

According to a preferred embodiment, exactly one of Z¹¹, Z¹², Z¹³, Z¹⁴, Z¹⁵, Z¹⁶, Z¹⁷, and Z¹⁸ per formula is CR¹¹. Furthermore preferably, in this case, all other of Z¹¹, Z¹², Z¹³, Z¹⁴, Z¹⁵, Z¹⁶, Z¹⁷, and Z¹⁸ are CR¹.

According to one preferred embodiment, one of Z¹¹, Z¹², Z¹³, Z¹⁴ is CR¹¹. According to another preferred embodiment, one of Z¹⁵, Z¹⁶, Z¹⁷, Z¹⁸ is CR¹¹.

Preferably, the bond drawn into the ring comprising Z¹¹, Z¹², Z¹³ and Z¹⁴ is attached to one of Z¹² and Z¹³.

Preferably Y is O.

Preferably, groups Ar¹ are selected, identically or differently, from phenyl, biphenyl, terphenyl, and quaterphenyl, which each bear no substituents, and fluorenyl, which is attached in one of its positions 1 to 4, and which is substituted with radicals R². Even more preferably, groups Ar¹ are selected, identically or differently, from biphenyl, which bears no substituents, and fluorenyl, which is attached in one of its positions 1 to 4 and which is substituted with radicals R². “Bear no substituents” means in the present case that the rings have only H bonded to them. Most preferably, both groups Ar¹ are biphenyl, which bears no substituents; or both groups Ar¹ are fluorenyl, which is attached in one of its positions 1 to 4 and which is substituted with radicals R². “Biphenyl” in this case is preferably para-biphenyl, and “fluorenyl, which is attached in one of its positions 1 to 4” is preferably 2-fluorenyl.

Highly preferred groups Ar¹ according to the present application are shown in the following:

| | |
|---|---|
| 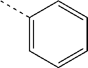 | Ar¹-1 |
| 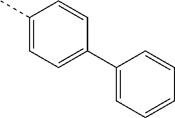 | Ar¹-2 |
| 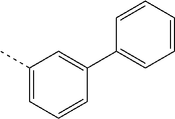 | Ar¹-3 |
| 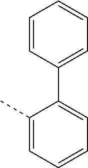 | Ar¹-4 |
| 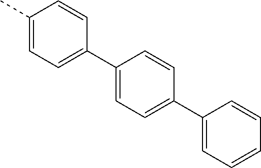 | Ar¹-5 |
| 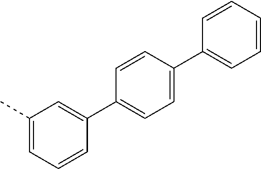 | Ar¹-6 |
| 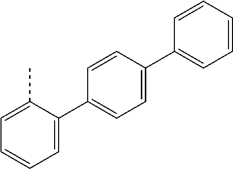 | Ar¹-7 |
| 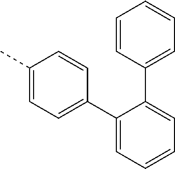 | Ar¹-8 |
| 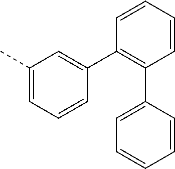 | Ar¹-9 |
| 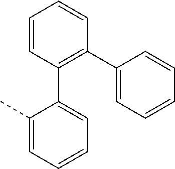 | Ar¹-10 |
| 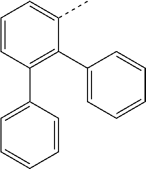 | Ar¹-11 |
| 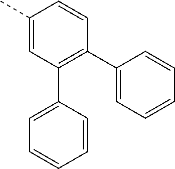 | Ar¹-12 |
| 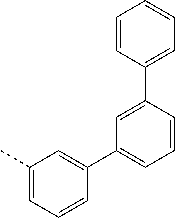 | Ar¹-13 |
| 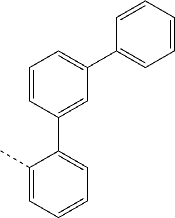 | Ar¹-14 |
| 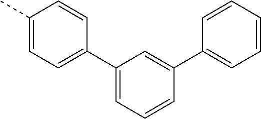 | Ar¹-15 |
| 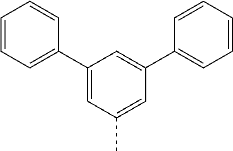 | Ar¹-16 |
| 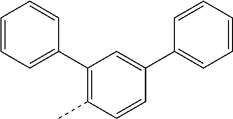 | Ar¹-17 |
| 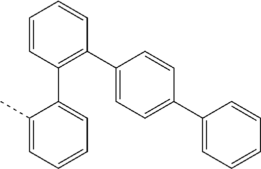 | Ar¹-18 |
| 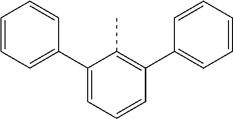 | Ar¹-19 |
| 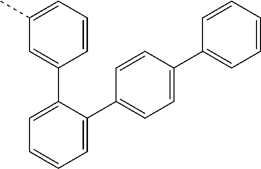 | Ar¹-20 |
| 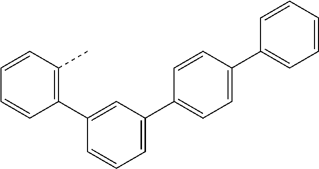 | Ar¹-21 |
| 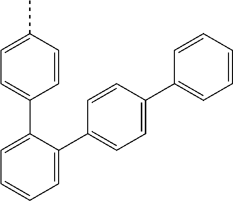 | Ar¹-22 |
| 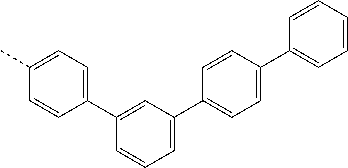 | Ar¹-23 |
| 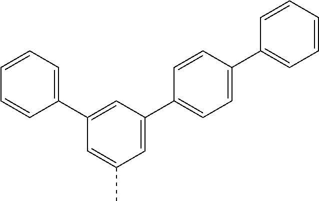 | Ar¹-24 |
| 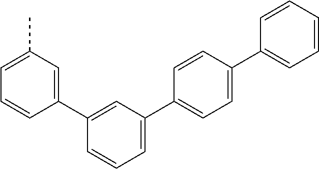 | Ar¹-25 |
| 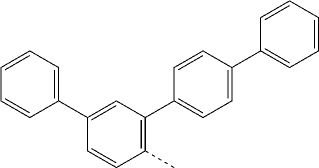 | Ar¹-26 |
| 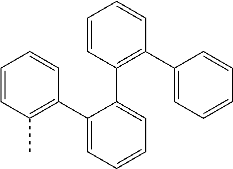 | Ar¹-27 |
| 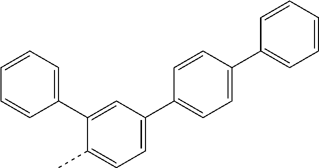 | Ar¹-28 |
| 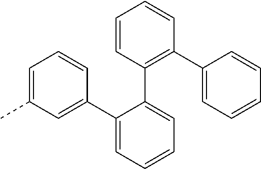 | Ar¹-29 |
| 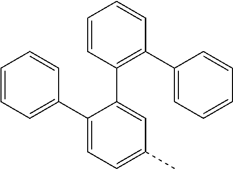 | Ar¹-30 |
| 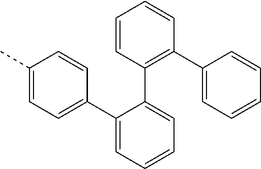 | Ar¹-31 |
| 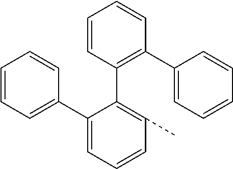 | Ar¹-32 |
| 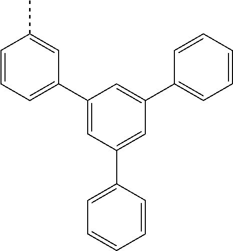 | Ar¹-33 |
| 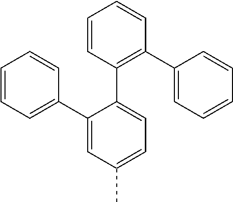 | Ar¹-34 |
| 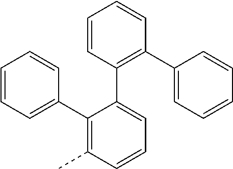 | Ar¹-35 |
| 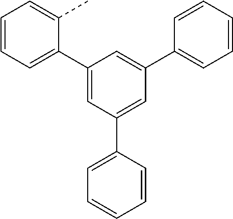 | Ar¹-36 |
| 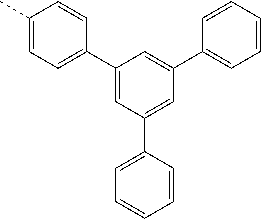 | Ar¹-37 |
| 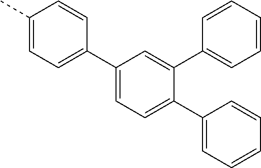 | Ar¹-38 |
| 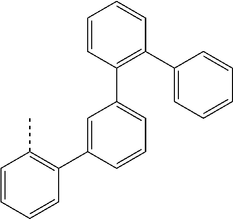 | Ar¹-39 |
| 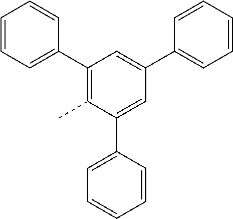 | Ar¹-40 |
| 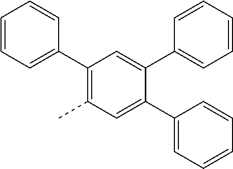 | Ar¹-41 |
| 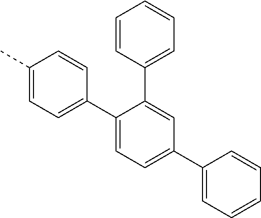 | Ar¹-42 |
| 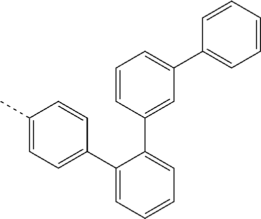 | Ar¹-43 |
| 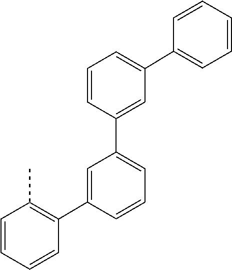 | Ar¹-44 |
| 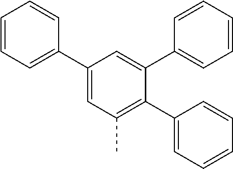 | Ar¹-45 |
| 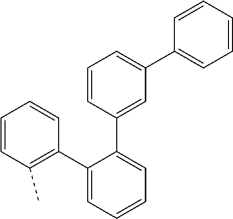 | Ar¹-46 |
| 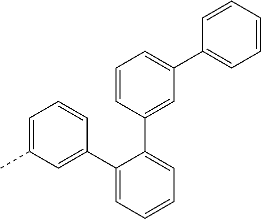 | Ar¹-47 |
| 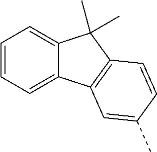 | Ar¹-139 |
| 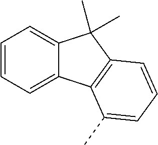 | Ar¹-140 |
| 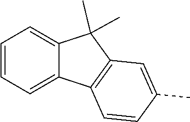 | Ar¹-141 |
| 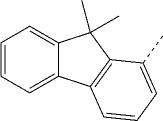 | Ar¹-142 |
| 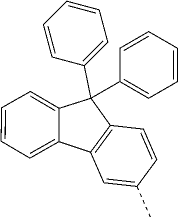 | Ar¹-172 |
| 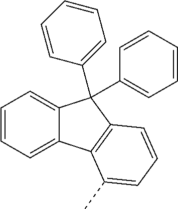 | Ar¹-173 |
| 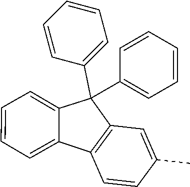 | Ar¹-174 |
| 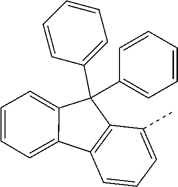 | Ar¹-177 |
| 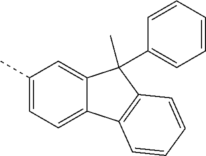 | Ar¹-195 |
| 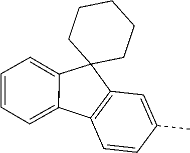 | Ar¹-199 |
| 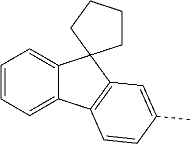 | Ar¹-200 |
| 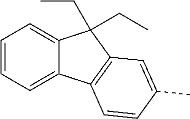 | Ar¹-201 |
| 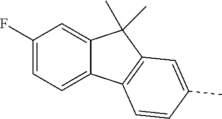 | Ar¹-202 |
| 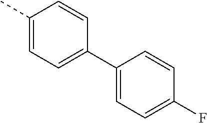 | Ar¹-238 |
| 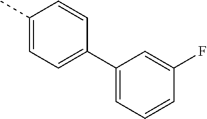 | Ar¹-239 |
| 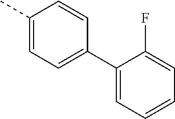 | Ar¹-240 |
| 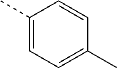 | Ar¹-241 |
| 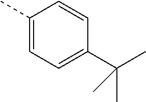 | Ar¹-242 |
| 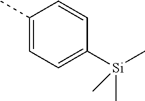 | Ar¹-243 |
| 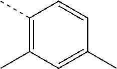 | Ar¹-244 |
| 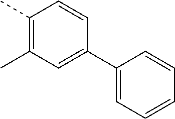 | Ar¹-245 |
| 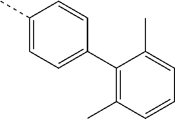 | Ar¹-246 |
| 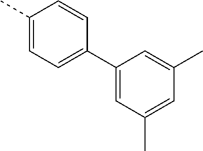 | Ar¹-247 |
| 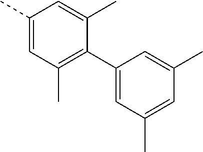 | Ar¹-248 |
| 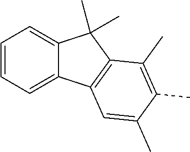 | Ar¹-249 |
| 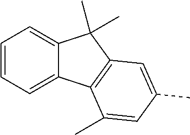 | Ar¹-250 |
| 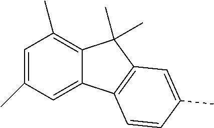 | Ar¹-251 |
| 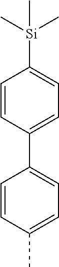 | Ar¹-252 |
| 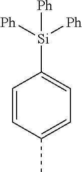 | Ar¹-253 |
| 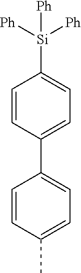 | Ar¹-254 |
| 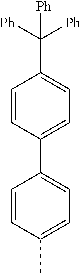 | Ar¹-255 |
| 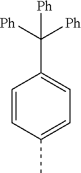 | Ar¹-256 |
| 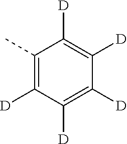 | Ar¹-260 |
| 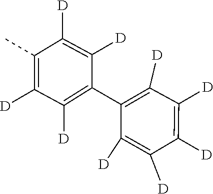 | Ar¹-262 |
| 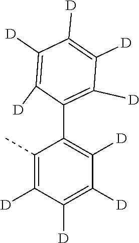 | Ar¹-264 |
| 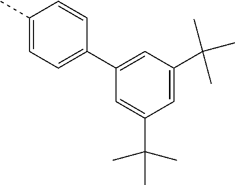 | Ar¹-265 |
| 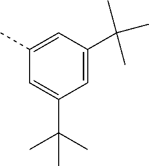 | Ar¹-266 |
| 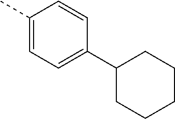 | Ar¹-267 |
| 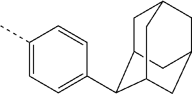 | Ar¹-268 |
| 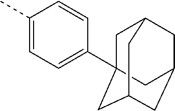 | Ar¹-269 |

Particularly preferred among these groups are: Ar¹-1 to Ar¹-7, Ar¹-15, Ar¹-17, Ar¹-41, Ar¹-42, Ar¹-139 to Ar¹-142, Ar¹-173, Ar¹-174, Ar¹-177, Ar¹-242, Ar¹-242, Ar¹-260, Ar¹-262, Ar¹-264 to Ar¹-266. Most preferred are Ar¹-2 and Ar¹-141.

Preferably, R¹ is selected, identically or differently, from H, D, F, CN, Si(R³)₃, N(R³)₂, straight-chain alkyl or alkoxy groups having 1 to 20 C atoms, branched or cyclic alkyl or alkoxy groups having 3 to 20 C atoms, aromatic ring systems having 6 to 40 aromatic ring atoms, and heteroaromatic ring systems having 5 to 40 aromatic ring atoms; where the said alkyl and alkoxy groups and the said aromatic and heteroaromatic ring systems are substituted by radicals R³, and where one or more CH₂ groups in the said alkyl and alkoxy groups may in each case be replaced by —C≡C—, —R³C═CR³, Si(R³)₂, C=O, C═NR³, —NR³, —O—, —S—, —C(═O)O— or —C(═O)NR³—. Particularly preferably, R¹ is selected, identically or differently, from H, D, straight-chain alkyl groups having 1 to 20 C atoms, branched or cyclic alkyl groups having 3 to 20 C atoms, aromatic ring systems having 6 to 40 aromatic ring atoms, which are substituted by radicals R³, and heteroaromatic ring systems having 5 to 40 aromatic ring atoms, which are substituted by radicals R³. Even more preferably, R¹ is selected identically or differently from H and D. Most preferably, R¹ is H.

According to a preferred embodiment, R¹¹ is selected, identically or differently, from aromatic ring systems having 6 to 24 aromatic ring atoms, which are substituted by radicals R³, and heteroaryl groups having 6 to 18 aromatic ring atoms, which are substituted by radicals R³. Heteroaryl groups are in this case preferably selected from dibenzofuranyl, carbazolyl and dibenzothiophenyl, which are each substituted by radicals R³. More preferably, R¹¹ is selected, identically or differently, from aromatic ring systems having 6 to 24 aromatic ring atoms, which are substituted by radicals R³, most preferably from phenyl, biphenyl, terphenyl, and naphthyl, which are substituted by radicals R³. Radicals R³ in this case are preferably selected identically or differently from H, D, F, straight-chain alkyl groups having 1 to 20 C atoms, and branched or cyclic alkyl groups having 3 to 20 C atoms. Highest preference in this case for R¹¹ is for phenyl, phenyl substituted with one group selected from tert-butyl group and OCF₃ group, phenyl substituted with two tert-butyl groups, biphenyl, naphthyl, biphenyl substituted with one or two tert-butyl groups, and dibenzofuranyl group.

Preferably, there is exactly one R¹¹ group present in the formula. Even more preferably, there is no further substituent on the dibenzofuran or dibenzothiophene moiety of the structure of formula (I), i.e. those groups Z¹¹ to Z¹⁸ which do not bear the group R¹¹ are CR¹, with R¹ being identically or differently H or D, most preferably being H.

Preferably, R² is selected, identically or differently, from H, D, F, CN, Si(R³)₃, N(R³)₂, straight-chain alkyl or alkoxy groups having 1 to 20 C atoms, branched or cyclic alkyl or alkoxy groups having 3 to 20 C atoms, aromatic ring systems having 6 to 40 aromatic ring atoms, and heteroaromatic ring systems having 5 to 40 aromatic ring atoms; where the said alkyl and alkoxy groups and the said aromatic and heteroaromatic ring systems are substituted by radicals R³, and where one or more CH₂ groups in the said alkyl and alkoxy groups may in each case be replaced by —C≡C—, —R³C═CR³—, Si(R³)₂, C═O, C═NR³, —NR³—, —O—, —S—, —C(═O)O— or —C(═O)NR³. Particularly preferably, R² is selected identically or differently from H, D, F, straight-chain alkyl groups having 1 to 20 C atoms, branched or cyclic alkyl groups having 3 to 20 C atoms, aromatic ring systems having 6 to 40 aromatic ring atoms, which are substituted by radicals R³, and heteroaromatic ring systems having 5 to 40 aromatic ring atoms, which are substituted by radicals R³. Furthermore preferably, R² which are on the aromatic rings of the fluorenyl group which is Ar¹ are, identically or differently, selected from H, D, F, straight-chain alkyl groups having 1 to 20 C atoms, and branched or cyclic alkyl groups having 3 to 20 C atoms; most preferably those groups are H or D, where H is preferred. Furthermore preferably, R² which are on the bridgehead aliphatic carbon atom of the fluorenyl group which is Ar¹ are, identically or differently, selected from F, straight-chain alkyl groups having 1 to 20 C atoms, branched or cyclic alkyl groups having 3 to 20 C atoms, and aromatic ring systems having 6 to 40 aromatic ring atoms, which are substituted by radicals R³.

Preferably, R²¹ is selected, identically or differently, from H, D, F, CN, Si(R³)₃, N(R³)₂, straight-chain alkyl or alkoxy groups having 1 to 20 C atoms, branched or cyclic alkyl or alkoxy groups having 3 to 20 C atoms; where the said alkyl and alkoxy groups are substituted by radicals R³, and where one or more CH₂ groups in the said alkyl and alkoxy groups may in each case be replaced by —C≡C—, —R³C═CR³—, Si(R³)₂, C═O, C═NR³, —NR³—, —O—, —S—, —C(═O)O— or —C(═O)NR³—. More preferably, R²¹ is selected identically or differently from H, D, F straight-chain alkyl groups having 1 to 20 C atoms, which are substituted by radicals R³, and branched or cyclic alkyl groups having 3 to 20 C atoms, which are substituted by radicals R³. Even more preferably, R²¹ is selected identically or differently from H and D. Most preferably, R²¹ is H.

Preferably, R³ is selected, identically or differently, from H, D, F, CN, Si(R⁴)₃, N(R⁴)₂, straight-chain alkyl or alkoxy groups having 1 to 20 C atoms, branched or cyclic alkyl or alkoxy groups having 3 to 20 C atoms, aromatic ring systems having 6 to 40 aromatic ring atoms, and heteroaromatic ring systems having 5 to 40 aromatic ring atoms; where the said alkyl and alkoxy groups and the said aromatic and heteroaromatic ring systems are substituted by radicals R⁴. Particularly preferably, R³ is selected identically or differently from H and D. Most preferably, R³ is H.

Preferably, R⁴ is selected, identically or differently, from H, D, F, CN, alkyl groups having 1 to 20 C atoms, aromatic ring systems having 6 to 40 C atoms, and heteroaromatic ring systems having 5 to 40 aromatic ring atoms; where the said alkyl groups, aromatic ring systems and heteroaromatic ring systems may be substituted by one or more radicals selected from F and CN.

Preferably, the compound fulfils the following conditions:
the compound conforms to formula (I), preferably to one of formulae (I-B-a), (I-B-b), (I-C-a) and (I-C-b); and
Y is O; and
Z¹ is CR¹; and
Z¹¹, Z¹², Z¹³, Z¹⁴ is each selected, identically or differently, from CR¹ and CR¹¹, if the bond drawn into the ring comprising Z¹¹, Z¹², Z¹³ and Z¹⁴ is not attached to it, and Z¹¹, Z¹², Z¹³, Z¹⁴ is C, if the bond drawn into the ring comprising Z¹¹, Z¹², Z¹³, Z¹⁴ is attached to it; and
Z¹⁵, Z¹⁶, Z¹⁷, Z¹⁸ is selected, identically or differently, from CR¹ and CR¹¹; and
exactly one of Z¹¹, Z¹², Z¹³, Z¹⁴, Z¹⁵, Z¹⁶, Z¹⁷ and Z¹⁸ is CR¹¹; and
Ar¹ is selected, identically or differently, from phenyl, biphenyl, terphenyl, and quaterphenyl, which each bear no substituents, and fluorenyl, which is attached in one of its positions 1 to 4, and which is substituted with radicals R²; and
R¹ is selected, identically or differently from H, D, straight-chain alkyl groups having 1 to 20 C atoms, branched or cyclic alkyl groups having 3 to 20 C atoms, aromatic ring systems having 6 to 40 aromatic ring atoms, which are substituted by radicals R³, and heteroaromatic ring systems having 5 to 40 aromatic ring atoms, which are substituted by radicals R³; and
R¹¹ is selected, identically or differently, from phenyl, biphenyl, terphenyl, and naphthyl, which are substituted by radicals R³, where R³ is selected identically or differently from H, D, F, straight-chain alkyl groups having 1 to 20 C atoms, and branched or cyclic alkyl groups having 3 to 20 C atoms; and
R² is selected identically or differently from H, D, F, straight-chain alkyl groups having 1 to 20 C atoms, branched or cyclic alkyl groups having 3 to 20 C atoms, aromatic ring systems having 6 to 40 aromatic ring atoms, which are substituted by radicals R³, and heteroaromatic ring systems having 5 to 40 aromatic ring atoms, which are substituted by radicals R³; and
R³ is selected, identically or differently, from H, D, F, CN, Si(R⁴)₃, N(R⁴)₂, straight-chain alkyl or alkoxy groups having 1 to 20 C atoms, branched or cyclic alkyl or alkoxy groups having 3 to 20 C atoms, aromatic ring systems having 6 to 40 aromatic ring atoms, and heteroaromatic ring systems having 5 to 40 aromatic ring atoms; where the said alkyl and alkoxy groups and the said aromatic and heteroaromatic ring systems are substituted by radicals R⁴; and
R⁴ is selected, identically or differently, from H, D, F, CN, alkyl groups having 1 to 20 C atoms, aromatic ring systems having 6 to 40 C atoms, and heteroaromatic ring systems having 5 to 40 aromatic ring atoms; where the said alkyl groups, aromatic ring systems and heteroaromatic ring systems may be substituted by one or more radicals selected from F and CN.

Preferred compounds according to formula (I) are listed in the following:

| | |
|---|---|
| 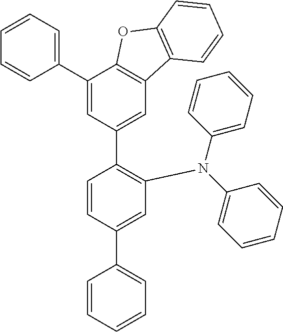 [SMILES: c1ccc(-c2ccc(-c3cc(-c4ccccc4)c4oc5ccccc5c4c3)c(N(c3ccccc3)c3ccccc3)c2)cc1] | 1 |
| 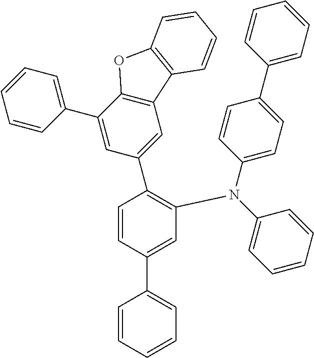 [SMILES: c1ccc(-c2ccc(N(c3ccccc3)c3cc(-c4ccccc4)ccc3-c3cc(-c4ccccc4)c4oc5ccccc5c4c3)cc2)cc1] | 2 |
| 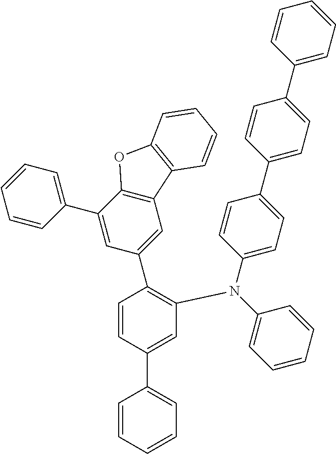 [SMILES: c1ccc(-c2ccc(-c3ccc(N(c4ccccc4)c4cc(-c5ccccc5)ccc4-c4cc(-c5ccccc5)c5oc6ccccc6c5c4)cc3)cc2)cc1] | 3 |
| 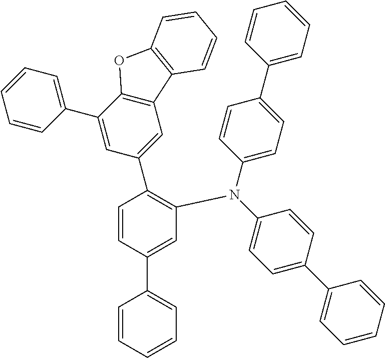 [SMILES: c1ccc(-c2ccc(N(c3ccc(-c4ccccc4)cc3)c3cc(-c4ccccc4)ccc3-c3cc(-c4ccccc4)c4oc5ccccc5c4c3)cc2)cc1] | 4 |
| 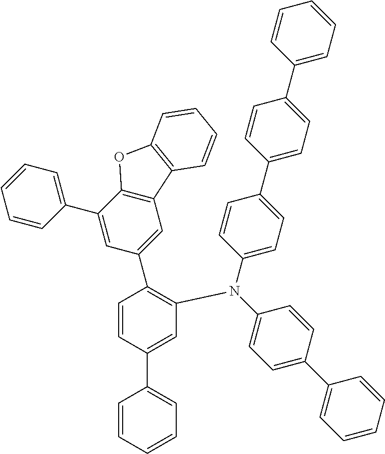 [SMILES: c1ccc(-c2ccc(-c3ccc(N(c4ccc(-c5ccccc5)cc4)c4cc(-c5ccccc5)ccc4-c4cc(-c5ccccc5)c5oc6ccccc6c5c4)cc3)cc2)cc1] | 5 |
| 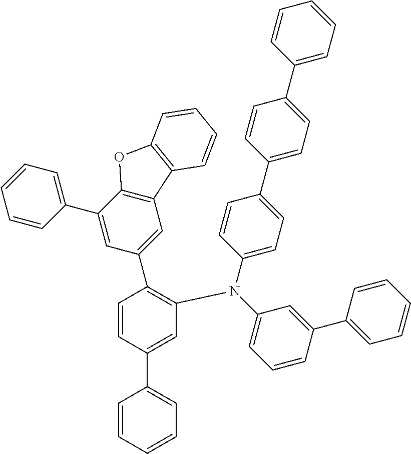 [SMILES: c1ccc(-c2ccc(-c3ccc(N(c4cccc(-c5ccccc5)c4)c4cc(-c5ccccc5)ccc4-c4cc(-c5ccccc5)c5oc6ccccc6c5c4)cc3)cc2)cc1] | 6 |
| 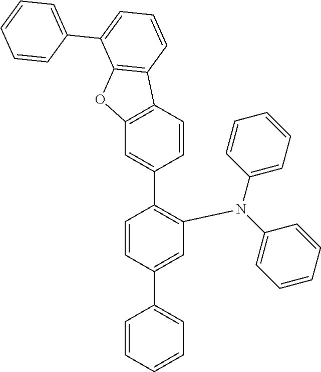 [SMILES: c1ccc(-c2ccc(-c3ccc4c(c3)oc3c(-c5ccccc5)cccc34)c(N(c3ccccc3)c3ccccc3)c2)cc1] | 7 |
| 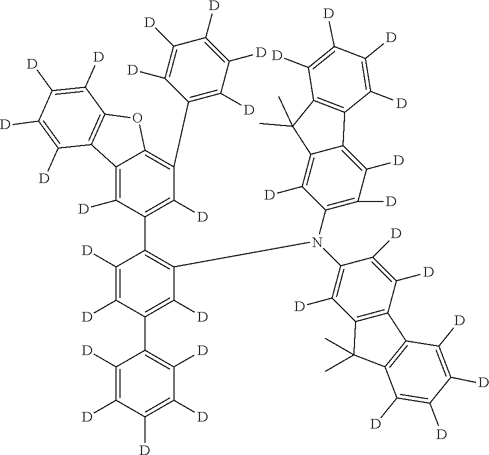 [SMILES: [2H]c1c([2H])c([2H])c(-c2c([2H])c([2H])c(-c3c([2H])c(-c4c([2H])c([2H])c([2H])c([2H])c4[2H])c4oc5c([2H])c([2H])c([2H])c([2H])c5c4c3[2H])c(N(c3c([2H])c([2H])c4c(c3[2H])C(C)(C)c3c([2H])c([2H])c([2H])c([2H])c3-4)c3c([2H])c([2H])c4c(c3[2H])C(C)(C)c3c([2H])c([2H])c([2H])c([2H])c3-4)c2[2H])c([2H])c1[2H]] | 8 |
| 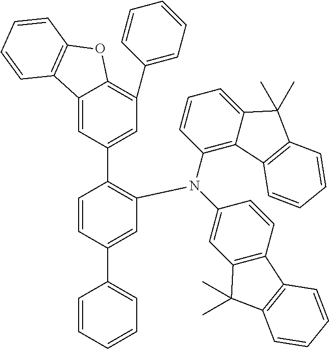 [SMILES: CC1(C)c2ccccc2-c2ccc(N(c3cc(-c4ccccc4)ccc3-c3cc(-c4ccccc4)c4oc5ccccc5c4c3)c3cccc4c3-c3ccccc3C4(C)C)cc21] | 9 |
| 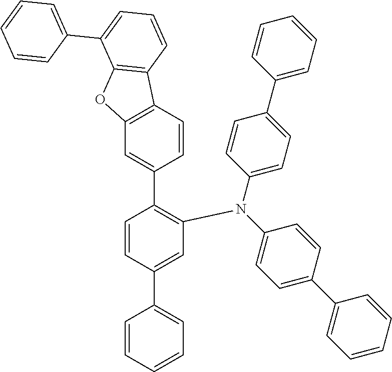 [SMILES: c1ccc(-c2ccc(N(c3ccc(-c4ccccc4)cc3)c3cc(-c4ccccc4)ccc3-c3ccc4c(c3)oc3c(-c5ccccc5)cccc34)cc2)cc1] | 10 |
| 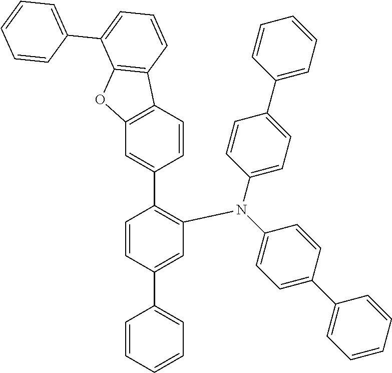 [SMILES: c1ccc(-c2ccc(N(c3ccc(-c4ccccc4)cc3)c3cc(-c4ccccc4)ccc3-c3ccc4c(c3)oc3c(-c5ccccc5)cccc34)cc2)cc1] | 11 |
| 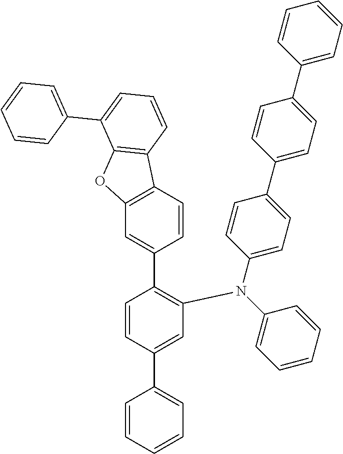 [SMILES: c1ccc(-c2ccc(-c3ccc(N(c4ccccc4)c4cc(-c5ccccc5)ccc4-c4ccc5c(c4)oc4c(-c6ccccc6)cccc45)cc3)cc2)cc1] | 12 |
| 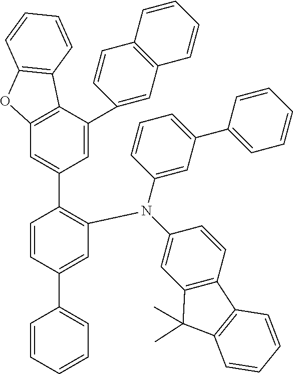 [SMILES: CC1(C)c2ccccc2-c2ccc(N(c3cccc(-c4ccccc4)c3)c3cc(-c4ccccc4)ccc3-c3cc(-c4ccc5ccccc5c4)c4c(c3)oc3ccccc34)cc21] | 13 |
| 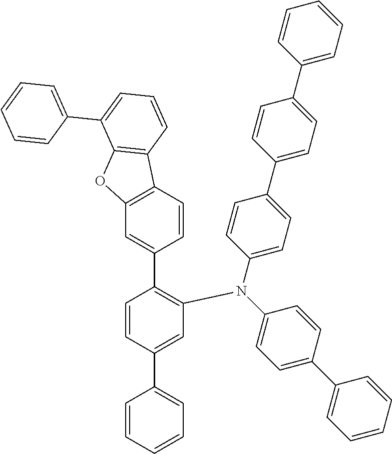 [SMILES: c1ccc(-c2ccc(-c3ccc(N(c4ccc(-c5ccccc5)cc4)c4cc(-c5ccccc5)ccc4-c4ccc5c(c4)oc4c(-c6ccccc6)cccc45)cc3)cc2)cc1] | 14 |
| 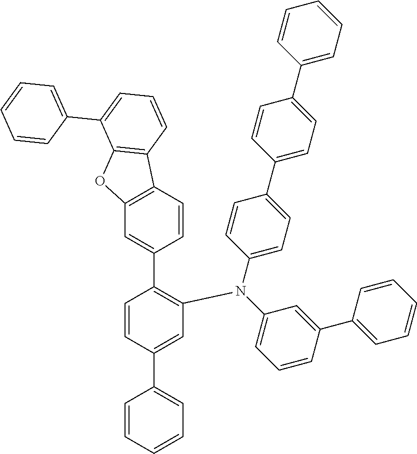 [SMILES: c1ccc(-c2ccc(-c3ccc(N(c4cccc(-c5ccccc5)c4)c4cc(-c5ccccc5)ccc4-c4ccc5c(c4)oc4c(-c6ccccc6)cccc45)cc3)cc2)cc1] | 15 |
| 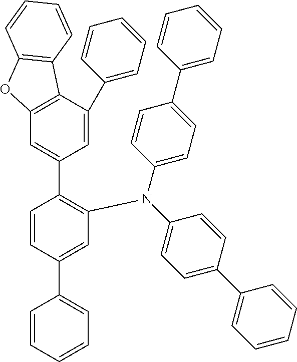 [SMILES: c1ccc(-c2ccc(N(c3ccc(-c4ccccc4)cc3)c3cc(-c4ccccc4)ccc3-c3cc(-c4ccccc4)c4c(c3)oc3ccccc34)cc2)cc1] | 16 |
| 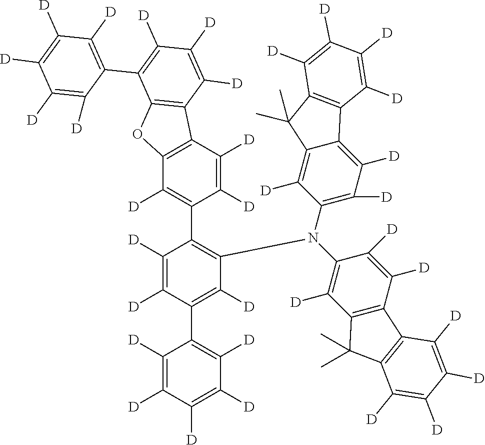 [SMILES: [2H]c1c([2H])c([2H])c(-c2c([2H])c([2H])c(-c3c([2H])c([2H])c4c(oc5c(-c6c([2H])c([2H])c([2H])c([2H])c6[2H])c([2H])c([2H])c([2H])c54)c3[2H])c(N(c3c([2H])c([2H])c4c(c3[2H])C(C)(C)c3c([2H])c([2H])c([2H])c([2H])c3-4)c3c([2H])c([2H])c4c(c3[2H])C(C)(C)c3c([2H])c([2H])c([2H])c([2H])c3-4)c2[2H])c([2H])c1[2H]] | 17 |
| 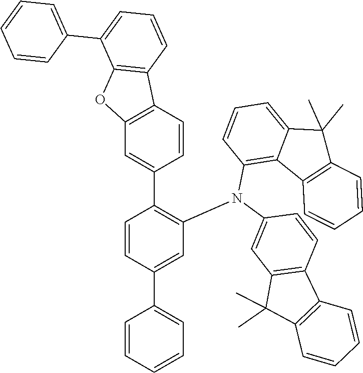 [SMILES: CC1(C)c2ccccc2-c2ccc(N(c3cc(-c4ccccc4)ccc3-c3ccc4c(c3)oc3c(-c5ccccc5)cccc34)c3cccc4c3-c3ccccc3C4(C)C)cc21] | 18 |
| 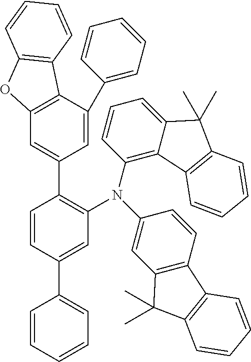 [SMILES: CC1(C)c2ccccc2-c2ccc(N(c3cc(-c4ccccc4)ccc3-c3cc(-c4ccccc4)c4c(c3)oc3ccccc34)c3cccc4c3-c3ccccc3C4(C)C)cc21] | 19 |
| 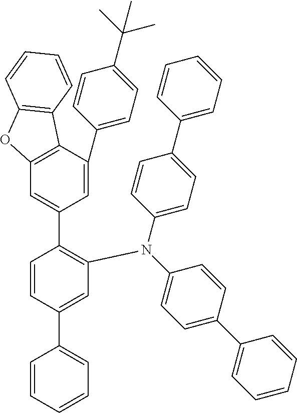 [SMILES: CC(C)(C)c1ccc(-c2cc(-c3ccc(-c4ccccc4)cc3N(c3ccc(-c4ccccc4)cc3)c3ccc(-c4ccccc4)cc3)cc3oc4ccccc4c23)cc1] | 20 |
| 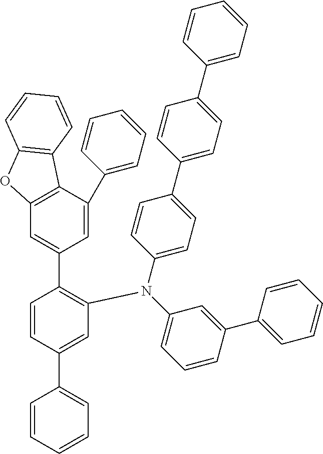 [SMILES: c1ccc(-c2ccc(-c3ccc(N(c4cccc(-c5ccccc5)c4)c4cc(-c5ccccc5)ccc4-c4cc(-c5ccccc5)c5c(c4)oc4ccccc45)cc3)cc2)cc1] | 21 |
| 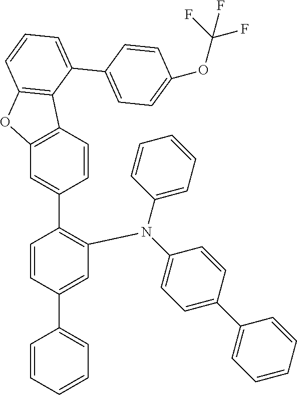 [SMILES: FC(F)(F)Oc1ccc(-c2cccc3oc4cc(-c5ccc(-c6ccccc6)cc5N(c5ccccc5)c5ccc(-c6ccccc6)cc5)ccc4c23)cc1] | 22 |
| 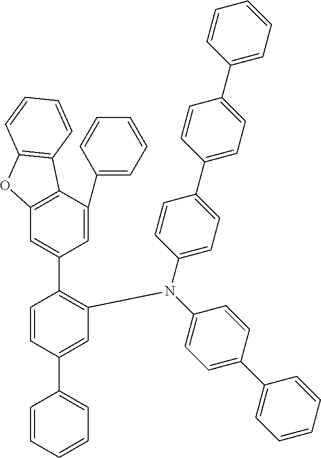 [SMILES: c1ccc(-c2ccc(-c3ccc(N(c4ccc(-c5ccccc5)cc4)c4cc(-c5ccccc5)ccc4-c4cc(-c5ccccc5)c5c(c4)oc4ccccc45)cc3)cc2)cc1] | 23 |
| 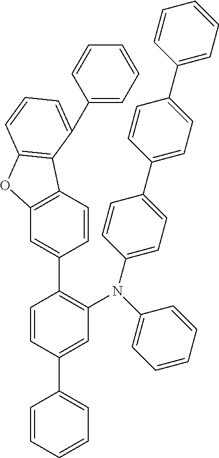 [SMILES: c1ccc(-c2ccc(-c3ccc(N(c4ccccc4)c4cc(-c5ccccc5)ccc4-c4ccc5c(c4)oc4cccc(-c6ccccc6)c45)cc3)cc2)cc1] | 24 |
| 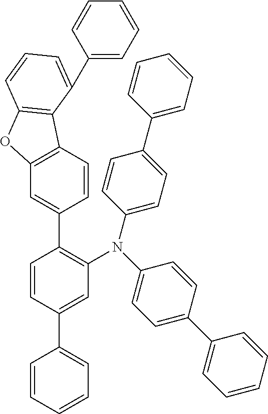 [SMILES: c1ccc(-c2ccc(N(c3ccc(-c4ccccc4)cc3)c3cc(-c4ccccc4)ccc3-c3ccc4c(c3)oc3cccc(-c5ccccc5)c34)cc2)cc1] | 25 |
| 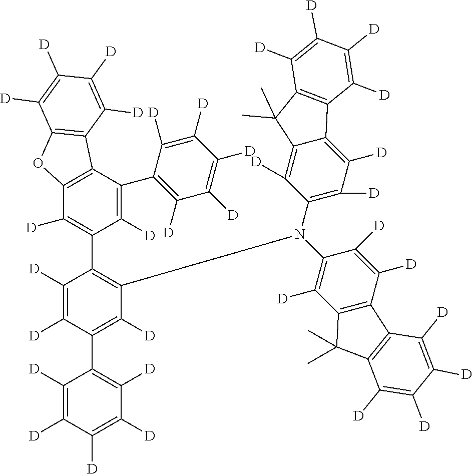 [SMILES: [2H]c1c([2H])c([2H])c(-c2c([2H])c([2H])c(-c3c([2H])c(-c4c([2H])c([2H])c([2H])c([2H])c4[2H])c4c(oc5c([2H])c([2H])c([2H])c([2H])c54)c3[2H])c(N(c3c([2H])c([2H])c4c(c3[2H])C(C)(C)c3c([2H])c([2H])c([2H])c([2H])c3-4)c3c([2H])c([2H])c4c(c3[2H])C(C)(C)c3c([2H])c([2H])c([2H])c([2H])c3-4)c2[2H])c([2H])c1[2H]] | 26 |
| 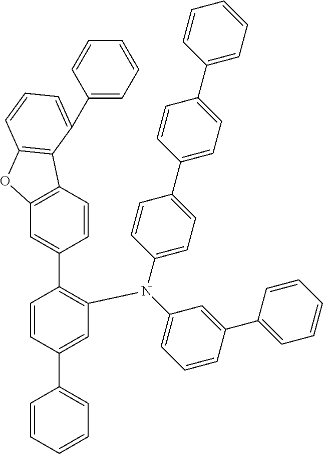 [SMILES: c1ccc(-c2ccc(-c3ccc(N(c4cccc(-c5ccccc5)c4)c4cc(-c5ccccc5)ccc4-c4ccc5c(c4)oc4cccc(-c6ccccc6)c45)cc3)cc2)cc1] | 27 |
| 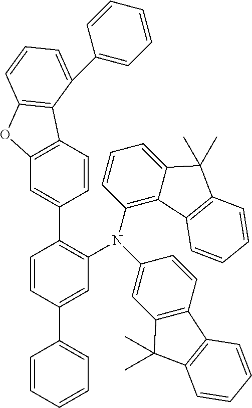 [SMILES: CC1(C)c2ccccc2-c2ccc(N(c3cc(-c4ccccc4)ccc3-c3ccc4c(c3)oc3cccc(-c5ccccc5)c34)c3cccc4c3-c3ccccc3C4(C)C)cc21] | 28 |
| 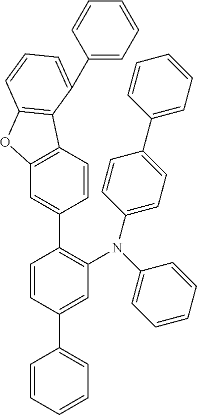 [SMILES: c1ccc(-c2ccc(N(c3ccccc3)c3cc(-c4ccccc4)ccc3-c3ccc4c(c3)oc3cccc(-c5ccccc5)c34)cc2)cc1] | 29 |
| 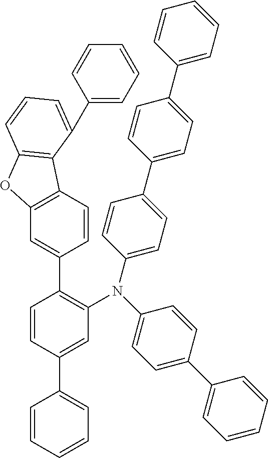 [SMILES: c1ccc(-c2ccc(-c3ccc(N(c4ccc(-c5ccccc5)cc4)c4cc(-c5ccccc5)ccc4-c4ccc5c(c4)oc4cccc(-c6ccccc6)c45)cc3)cc2)cc1] | 30 |
| 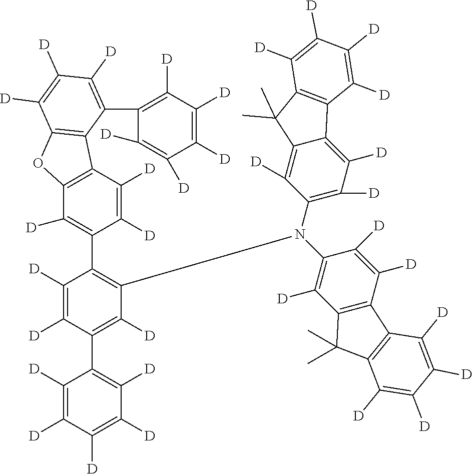 [SMILES: [2H]c1c([2H])c([2H])c(-c2c([2H])c([2H])c(-c3c([2H])c([2H])c4c(oc5c([2H])c([2H])c([2H])c(-c6c([2H])c([2H])c([2H])c([2H])c6[2H])c54)c3[2H])c(N(c3c([2H])c([2H])c4c(c3[2H])C(C)(C)c3c([2H])c([2H])c([2H])c([2H])c3-4)c3c([2H])c([2H])c4c(c3[2H])C(C)(C)c3c([2H])c([2H])c([2H])c([2H])c3-4)c2[2H])c([2H])c1[2H]] | 31 |
| 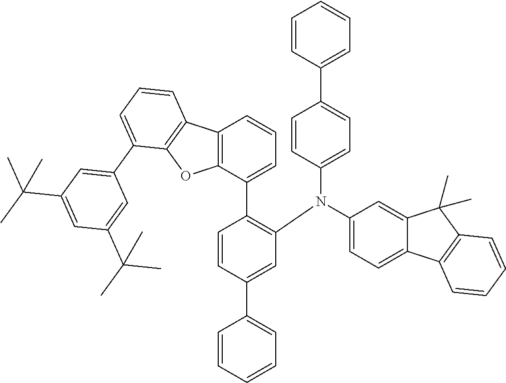 [SMILES: CC(C)(C)c1cc(-c2cccc3c2oc2c(-c4ccc(-c5ccccc5)cc4N(c4ccc(-c5ccccc5)cc4)c4ccc5c(c4)C(C)(C)c4ccccc4-5)cccc23)cc(C(C)(C)C)c1] | 32 |
| 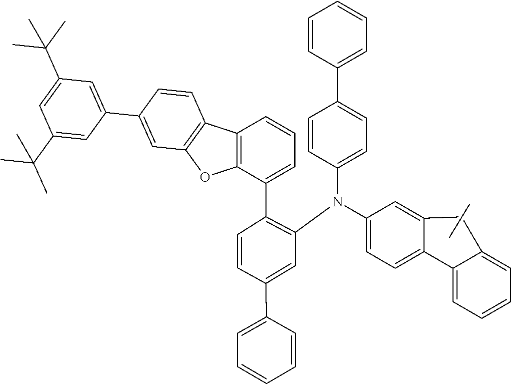 | 33 |
| 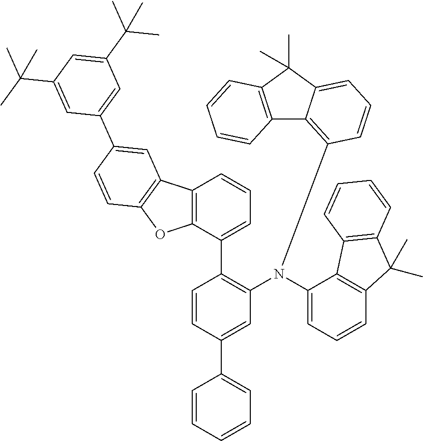 [SMILES: CC(C)(C)c1cc(-c2ccc3oc4c(-c5ccc(-c6ccccc6)cc5N(c5cccc6c5-c5ccccc5C6(C)C)c5cccc6c5-c5ccccc5C6(C)C)cccc4c3c2)cc(C(C)(C)C)c1] | 34 |
| 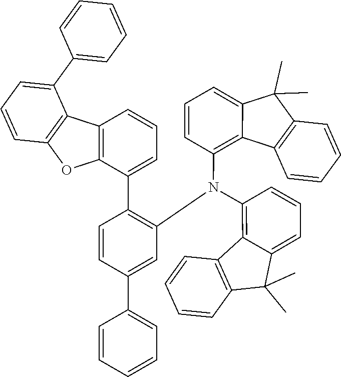 [SMILES: CC1(C)c2ccccc2-c2c(N(c3cc(-c4ccccc4)ccc3-c3cccc4c3oc3cccc(-c5ccccc5)c34)c3cccc4c3-c3ccccc3C4(C)C)cccc21] | 35 |
| 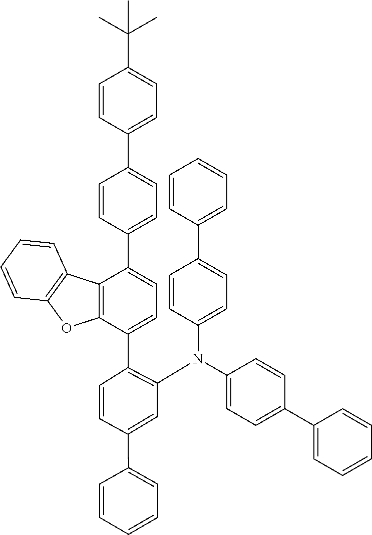 [SMILES: CC(C)(C)c1ccc(-c2ccc(-c3ccc(-c4ccc(-c5ccccc5)cc4N(c4ccc(-c5ccccc5)cc4)c4ccc(-c5ccccc5)cc4)c4oc5ccccc5c34)cc2)cc1] | 36 |
| 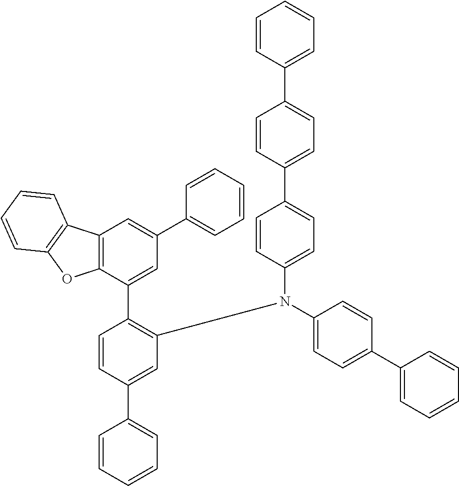 [SMILES: c1ccc(-c2ccc(-c3ccc(N(c4ccc(-c5ccccc5)cc4)c4cc(-c5ccccc5)ccc4-c4cc(-c5ccccc5)cc5c4oc4ccccc45)cc3)cc2)cc1] | 37 |
| 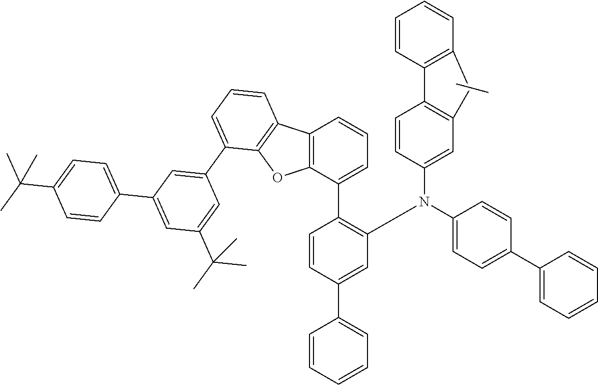 | 38 |
| 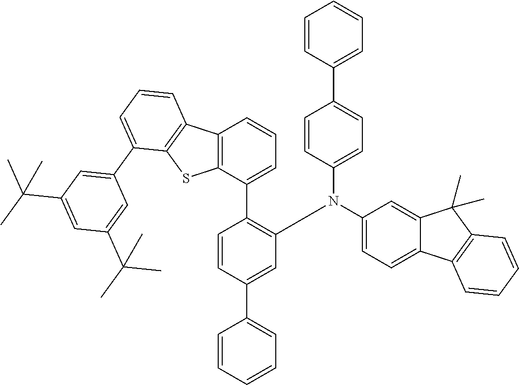 [SMILES: CC(C)(C)c1cc(-c2cccc3c2sc2c(-c4ccc(-c5ccccc5)cc4CN(c4ccc(-c5ccccc5)cc4)c4ccc5c(c4)C(C)(C)c4ccccc4-5)cccc23)cc(C(C)(C)C)c1] | 39 |
| 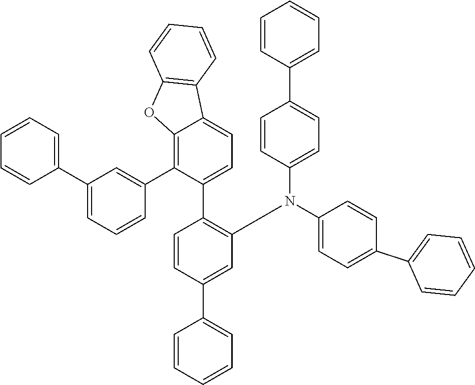 [SMILES: c1ccc(-c2ccc(N(Cc3cc(-c4ccccc4)ccc3-c3ccc4c(oc5ccccc54)c3-c3cccc(-c4ccccc4)c3)c3ccc(-c4ccccc4)cc3)cc2)cc1] | 40 |
| 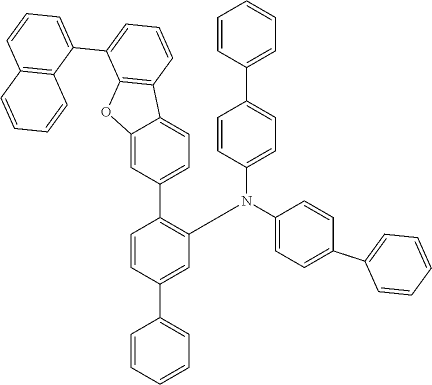 [SMILES: c1ccc(-c2ccc(N(Cc3cc(-c4ccccc4)ccc3-c3ccc4c(c3)oc3c(-c5cccc6ccccc56)cccc34)c3ccc(-c4ccccc4)cc3)cc2)cc1] | 41 |
| 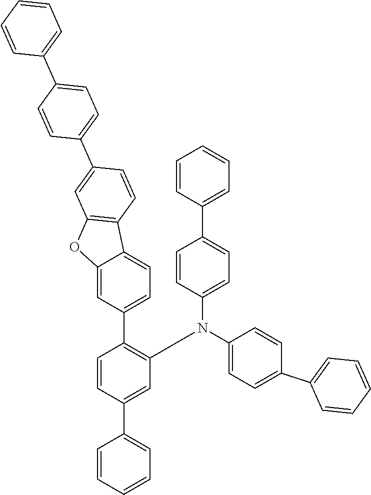 [SMILES: c1ccc(-c2ccc(-c3ccc4c(c3)oc3cc(-c5ccc(-c6ccccc6)cc5CN(c5ccc(-c6ccccc6)cc5)c5ccc(-c6ccccc6)cc5)ccc34)cc2)cc1] | 42 |
| 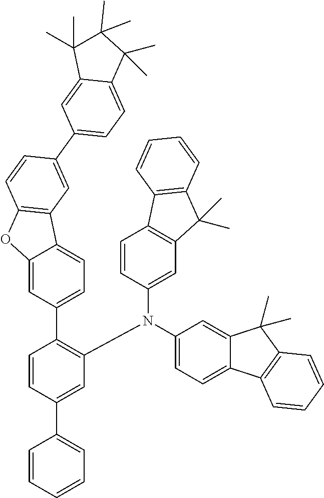 [SMILES: CC1(C)c2ccccc2-c2ccc(N(c3ccc4c(c3)C(C)(C)c3ccccc3-4)c3cc(-c4ccccc4)ccc3-c3ccc4c(c3)oc3ccc(-c5ccc6c(c5)C(C)(C)C(C)(C)C6(C)C)cc34)cc21] | 43 |
| 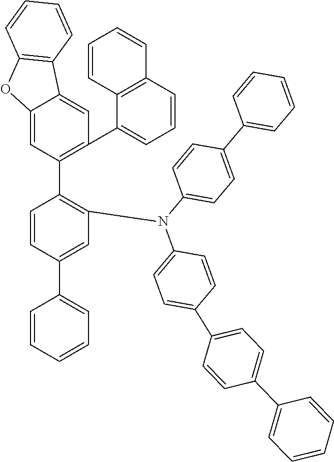 [SMILES: c1ccc(-c2ccc(-c3ccc(N(c4ccc(-c5ccccc5)cc4)c4cc(-c5ccccc5)ccc4-c4cc5oc6ccccc6c5cc4-c4cccc5ccccc45)cc3)cc2)cc1] | 44 |
| 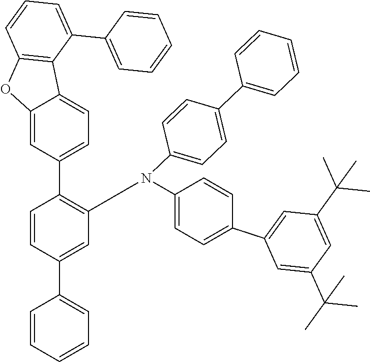 [SMILES: CC(C)(C)c1cc(-c2ccc(N(Cc3cc(-c4ccccc4)ccc3-c3ccc4c(c3)oc3cccc(-c5ccccc5)c34)c3ccc(-c4ccccc4)cc3)cc2)cc(C(C)(C)C)c1] | 45 |
| 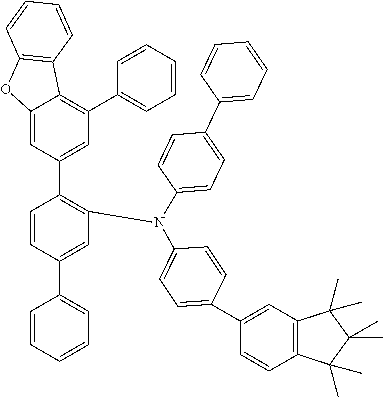 [SMILES: CC1(C)c2ccc(-c3ccc(N(c4ccc(-c5ccccc5)cc4)c4cc(-c5ccccc5)ccc4-c4cc(-c5ccccc5)c5c(c4)oc4ccccc45)cc3)cc2C(C)(C)C1(C)C] | 46 |
| 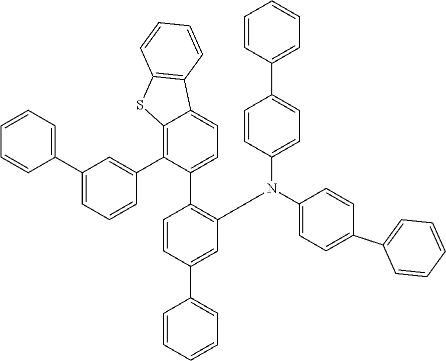 [SMILES: c1ccc(-c2ccc(N(Cc3cc(-c4ccccc4)ccc3-c3ccc4c(sc5ccccc54)c3-c3cccc(-c4ccccc4)c3)c3ccc(-c4ccccc4)cc3)cc2)cc1] | 47 |
| 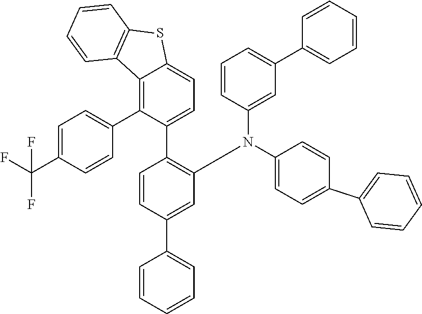 [SMILES: FC(F)(F)c1ccc(-c2c(-c3ccc(-c4ccccc4)cc3CN(c3ccc(-c4ccccc4)cc3)c3cccc(-c4ccccc4)c3)ccc3sc4ccccc4c23)cc1] | 48 |
| 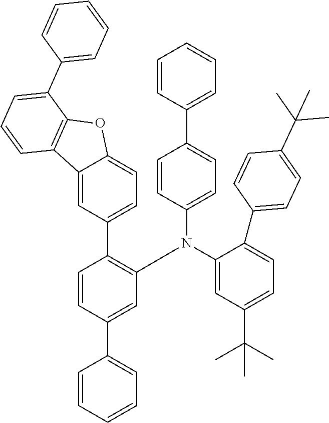 [SMILES: CC(C)(C)c1ccc(-c2ccc(C(C)(C)C)cc2N(c2ccc(-c3ccccc3)cc2)c2cc(-c3ccccc3)ccc2-c2ccc3oc4c(-c5ccccc5)cccc4c3c2)cc1] | 49 |
| 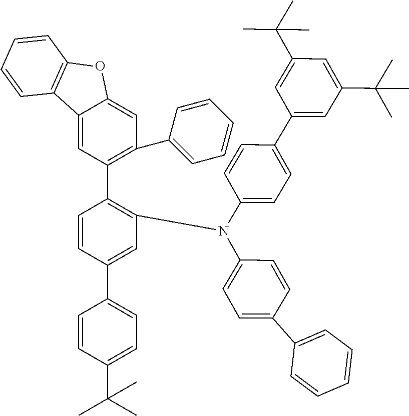 [SMILES: CC(C)(C)c1ccc(-c2ccc(-c3cc4c(cc3-c3ccccc3)oc3ccccc34)c(N(c3ccc(-c4ccccc4)cc3)c3ccc(-c4cc(C(C)(C)C)cc(C(C)(C)C)c4)cc3)c2)cc1] | 50 |
| 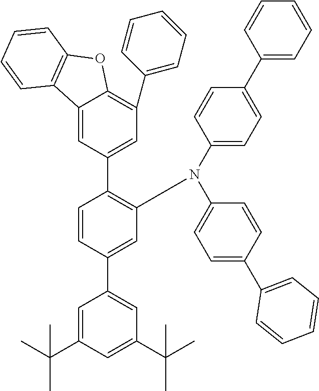 [SMILES: CC(C)(C)c1cc(-c2ccc(-c3cc(-c4ccccc4)c4oc5ccccc5c4c3)c(CN(c3ccc(-c4ccccc4)cc3)c3ccc(-c4ccccc4)cc3)c2)cc(C(C)(C)C)c1] | 51 |
| 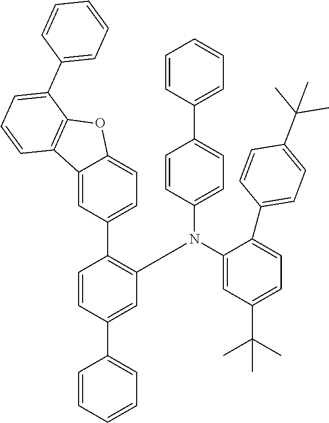 [SMILES: CC(C)(C)c1ccc(-c2ccc(C(C)(C)C)cc2N(Cc2cc(-c3ccccc3)ccc2-c2ccc3oc4c(-c5ccccc5)cccc4c3c2)c2ccc(-c3ccccc3)cc2)cc1] | 52 |
| 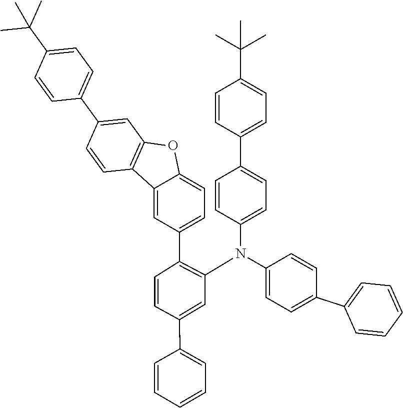 [SMILES: CC(C)(C)c1ccc(-c2ccc(N(c3ccc(-c4ccccc4)cc3)c3cc(-c4ccccc4)ccc3-c3ccc4oc5cc(-c6ccc(C(C)(C)C)cc6)ccc5c4c3)cc2)cc1] | 53 |
| 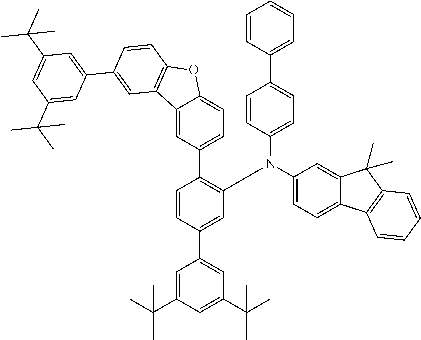 [SMILES: CC(C)(C)c1cc(-c2ccc(-c3ccc4oc5ccc(-c6cc(C(C)(C)C)cc(C(C)(C)C)c6)cc5c4c3)c(CN(c3ccc(-c4ccccc4)cc3)c3ccc4c(c3)C(C)(C)c3ccccc3-4)c2)cc(C(C)(C)C)c1] | 54 |
| 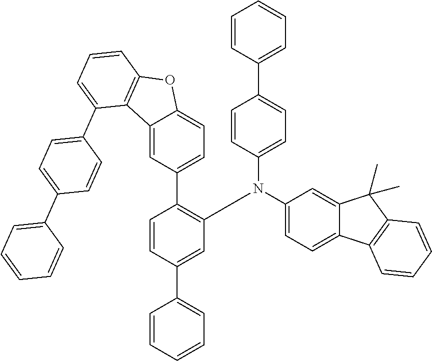 [SMILES: CC1(C)c2ccccc2-c2ccc(N(Cc3cc(-c4ccccc4)ccc3-c3ccc4oc5cccc(-c6ccc(-c7ccccc7)cc6)c5c4c3)c3ccc(-c4ccccc4)cc3)cc21] | 55 |
| 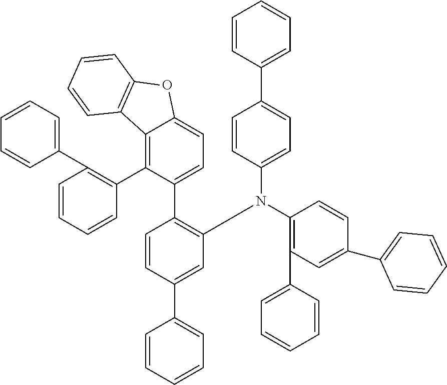 [SMILES: c1ccc(-c2ccc(N(Cc3cc(-c4ccccc4)ccc3-c3ccc4oc5ccccc5c4c3-c3ccccc3-c3ccccc3)c3ccc(-c4ccccc4)cc3-c3ccccc3)cc2)cc1] | 56 |
| 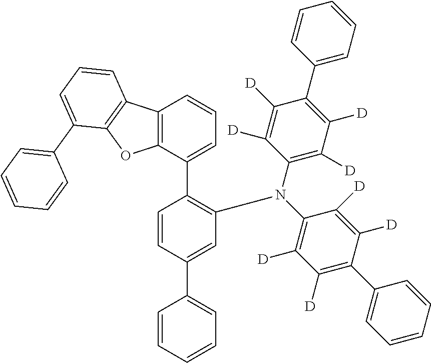 [SMILES: [2H]c1c([2H])c(N(Cc2cc(-c3ccccc3)ccc2-c2cccc3c2oc2c(-c4ccccc4)cccc23)c2c([2H])c([2H])c(-c3ccccc3)c([2H])c2[2H])c([2H])c([2H])c1-c1ccccc1] | 57 |
| 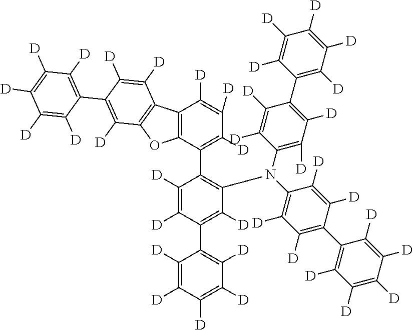 [SMILES: [2H]c1c([2H])c([2H])c(-c2c([2H])c([2H])c(N(Cc3c([2H])c(-c4c([2H])c([2H])c([2H])c([2H])c4[2H])c([2H])c([2H])c3-c3c([2H])c([2H])c([2H])c4c3oc3c([2H])c(-c5c([2H])c([2H])c([2H])c([2H])c5[2H])c([2H])c([2H])c34)c3c([2H])c([2H])c(-c4c([2H])c([2H])c([2H])c([2H])c4[2H])c([2H])c3[2H])c([2H])c2[2H])c([2H])c1[2H]] | 58 |
| 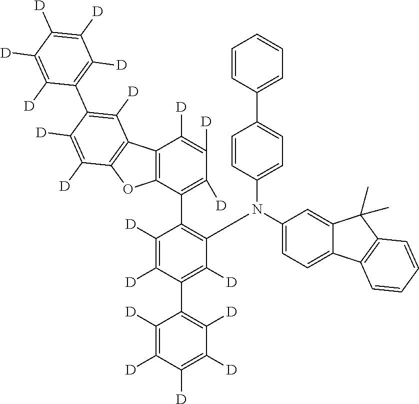 [SMILES: [2H]c1c([2H])c([2H])c(-c2c([2H])c([2H])c(-c3c([2H])c([2H])c([2H])c4c3oc3c([2H])c([2H])c(-c5c([2H])c([2H])c([2H])c([2H])c5[2H])c([2H])c34)c(CN(c3ccc(-c4ccccc4)cc3)c3ccc4c(c3)C(C)(C)c3ccccc3-4)c2[2H])c([2H])c1[2H]] | 59 |
| 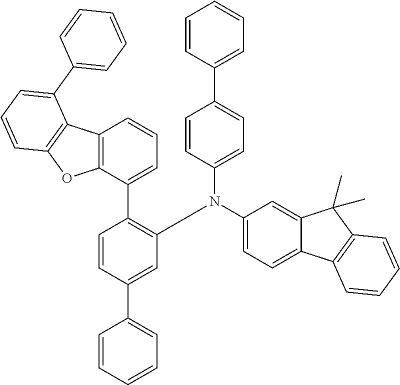 [SMILES: CC1(C)c2ccccc2-c2ccc(N(Cc3cc(-c4ccccc4)ccc3-c3cccc4c3oc3cccc(-c5ccccc5)c34)c3ccc(-c4ccccc4)cc3)cc21] | 60 |
| 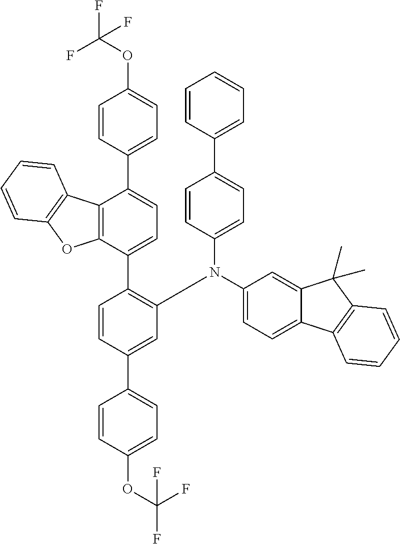 [SMILES: CC1(C)c2ccccc2-c2ccc(N(Cc3cc(-c4ccc(OC(F)(F)F)cc4)ccc3-c3ccc(-c4ccc(OC(F)(F)F)cc4)c4c3oc3ccccc34)c3ccc(-c4ccccc4)cc3)cc21] | 61 |
| 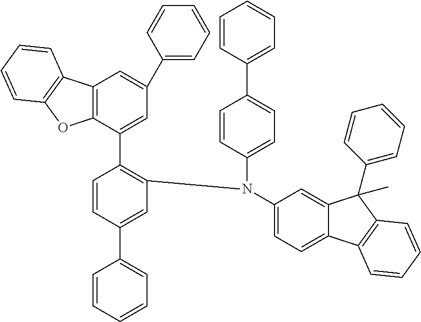 [SMILES: CC1(c2ccccc2)c2ccccc2-c2ccc(N(c3ccc(-c4ccccc4)cc3)c3cc(-c4ccccc4)ccc3-c3cc(-c4ccccc4)cc4c3oc3ccccc34)cc21] | 62 |
| 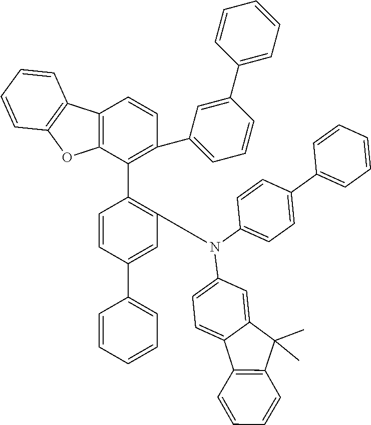 [SMILES: CC1(C)c2ccccc2-c2ccc(N(Cc3cc(-c4ccccc4)ccc3-c3c(-c4cccc(-c5ccccc5)c4)ccc4c3oc3ccccc34)c3ccc(-c4ccccc4)cc3)cc21] | 63 |
| 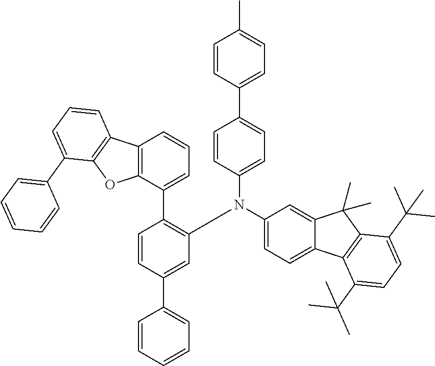 [SMILES: Cc1ccc(-c2ccc(N(Cc3cc(-c4ccccc4)ccc3-c3cccc4c3oc3c(-c5ccccc5)cccc34)c3ccc4c(c3)C(C)(C)c3c(C(C)(C)C)ccc(C(C)(C)C)c3-4)cc2)cc1] | 64 |
| 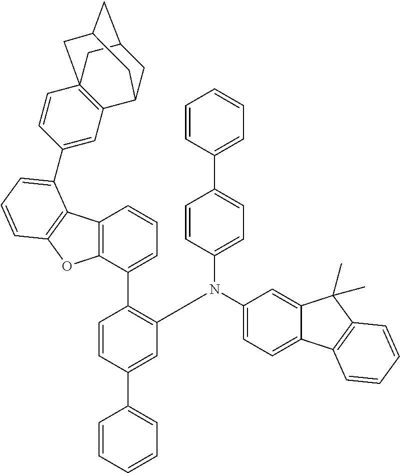 | 65 |
| 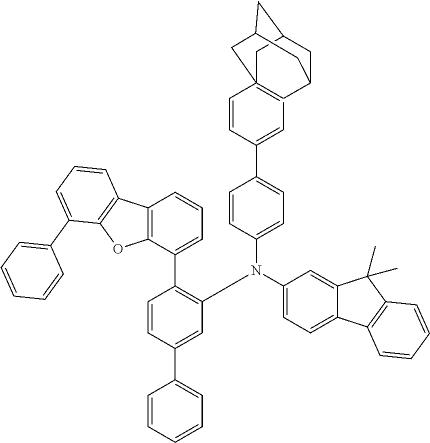 | 66 |
| 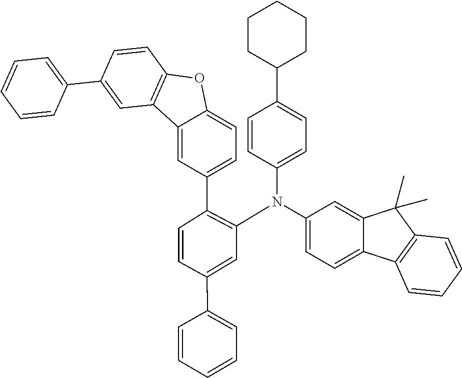 [SMILES: CC1(C)c2ccccc2-c2ccc(N(c3ccc(C4CCCCC4)cc3)c3cc(-c4ccccc4)ccc3-c3ccc4oc5ccc(-c6ccccc6)cc5c4c3)cc21] | 67 |
| 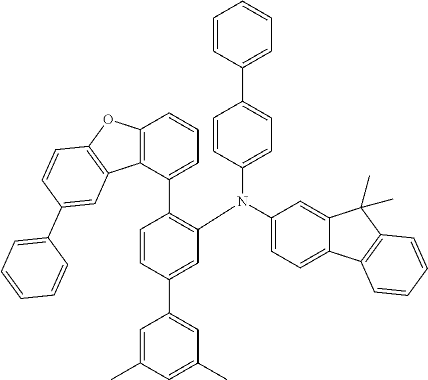 [SMILES: Cc1cc(C)cc(-c2ccc(-c3cccc4oc5ccc(-c6ccccc6)cc5c34)c(N(c3ccc(-c4ccccc4)cc3)c3ccc4c(c3)C(C)(C)c3ccccc3-4)c2)c1] | 68 |
| 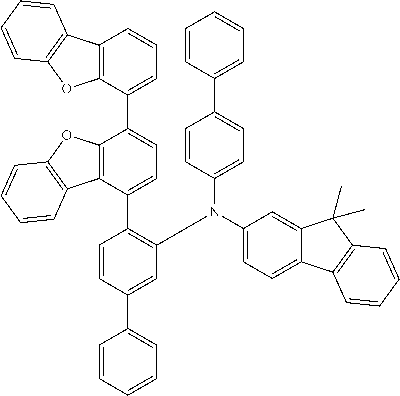 [SMILES: CC1(C)c2ccccc2-c2ccc(N(Cc3cc(-c4ccccc4)ccc3-c3ccc(-c4cccc5c4oc4ccccc45)c4oc5ccccc5c34)c3ccc(-c4ccccc4)cc3)cc21] | 69 |

The compounds according to formula (I) may be prepared by synthesis methods such as Buchwald coupling and Suzuki coupling. The skilled person is aware of several possible synthetic routes, based on his general knowledge of organic synthetic chemistry.

A preferred synthetic route to prepare compounds according to formula (I) is described in the following: For preparation of the compound according to formula (I), a biphenyl derivate with two reactive groups, one in 3- and one in 4-position on the biphenyl moiety, is first reacted with a dibenzofuranyl or dibenzothiophenyl group that bears a reactive group on one of its aromatic cores, and that bears an aromatic or heteroaromatic group as substituent, in a Suzuki coupling reaction. The reactive groups are preferably selected differently. The reaction takes place on the reactive group present in the 4-position of the biphenyl group. The resulting intermediate is then reacted further on the 3-position of the biphenyl moiety, in a Buchwald coupling reaction, with a secondary amine bearing two aromatic groups, to result in a compound of formula (I). Further reactions may follow. The intermediates described are in many cases commercially available. Specific intermediates needed to obtain specific compounds according to formula (I) that are not available commercially, can be prepared using methods known to the skilled person.

The above-mentioned synthetic scheme is illustrated in the following:

### Scheme 1

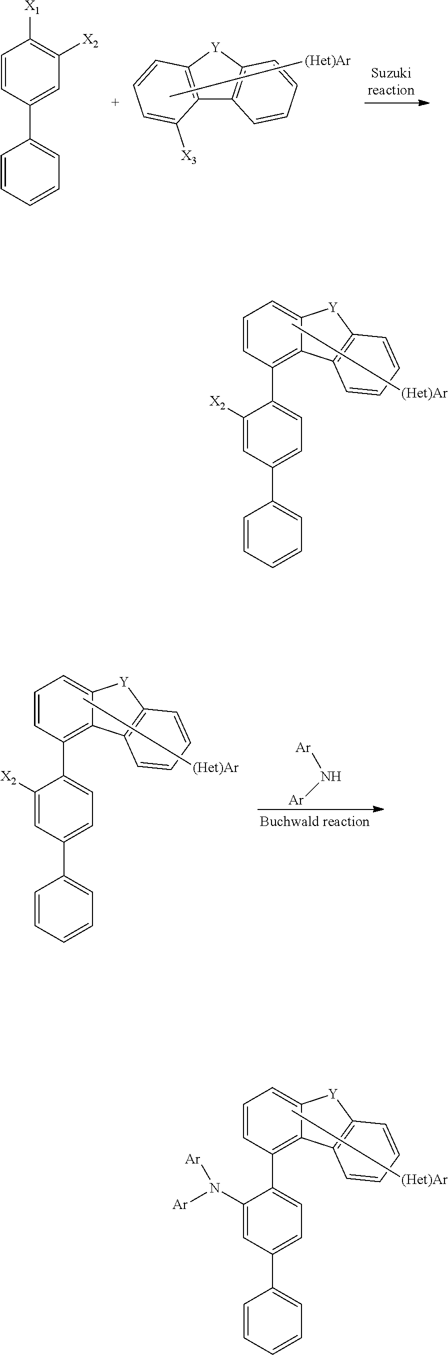

where the first step is in the first line, and the second step is in the second line, and
where the variable groups are defined as follows:
X₁, X₂ is identically or differently, preferably differently, selected from reactive groups, preferably Cl, Br, I, and sulfonyl groups;
X₃ is a reactive group, preferably boronic acid or boronic acid ester group;
Y is O or S;
R is an organic group, preferably an optionally substituted aromatic group;
(Het)Ar is an optionally substituted aromatic or heteroaromatic group;
Ar is selected, identically or differently, from optionally substituted aromatic groups.

Object of the present patent application is therefore a process for preparation of a compound according to formula (I), as defined above, where in a first step, a biphenyl derivate which bears two reactive groups, where one of the two reactive groups is present in the 3-position of the biphenyl derivate, and the other is present in the 4-position of the biphenyl derivate, is reacted with a dibenzofuranyl or dibenzothiophenyl derivate which bears a reactive group on one of its aromatic cores and which bears an aromatic or heteroaromatic group as substituent, in a Suzuki coupling reaction, where the reactive group in the 4-position reacts in the coupling reaction.

The reactive groups are preferably selected differently. The resulting intermediate is preferably then reacted further on the 3-position of the biphenyl moiety, in a Buchwald coupling reaction, with a secondary amine bearing two aromatic groups, to result in a compound of formula (I).

The above-described compounds of the invention, especially compounds substituted by reactive leaving groups, such as bromine, iodine, chlorine, boronic acid or boronic ester, may find use as monomers for production of corresponding oligomers, dendrimers or polymers. Suitable reactive leaving groups are, for example, bromine, iodine, chlorine, boronic acids, boronic esters, amines, alkenyl or alkynyl groups having a terminal C—C double bond or C—C triple bond, oxiranes, oxetanes, groups which enter into a cycloaddition, for example a 1,3-dipolar cycloaddition, for example dienes or azides, carboxylic acid derivatives, alcohols and silanes.

The invention therefore further provides oligomers, polymers or dendrimers containing one or more compounds of formula (I), wherein the bond(s) to the polymer, oligomer or dendrimer may be localized at any desired positions substituted by R¹, R¹¹, R², R²¹, R³ or R⁴ in formula (I). According to the linkage of the compound of formula (I), the compound is part of a side chain of the oligomer or polymer or part of the main chain.

Further technical details of such oligomers, polymers and dendrimers containing one or more compounds of formula (I) are as disclosed on p. 49, 1. 26-p. 51, 1. 17 of WO2020/109434A1. The cited disclosure is herewith incorporated into the present application in its entirety.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, alpha-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, or mixtures of these solvents.

The invention therefore further provides a formulation, especially a solution, dispersion or emulsion, comprising at least one compound of formula (I) or at least one polymer, oligomer or dendrimer containing at least one unit of formula (I) and at least one solvent, preferably an organic solvent. The way in which such solutions can be prepared is known to those skilled in the art.

The compound of formula (I) is suitable for use in an electronic device, especially an organic electroluminescent device (OLED). Depending on the substitution, the compound of the formula (I) can be used in different functions and layers. Preference is given to use as a hole-transporting material in a hole-transporting layer and/or as matrix material in an emitting layer, more preferably in combination with a phosphorescent emitter.

The invention therefore further provides for the use of a compound of formula (I) in an electronic device. This electronic device is preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and more preferably organic electroluminescent devices (OLEDs).

The invention further provides an electronic device comprising at least one compound of formula (I). This electronic device is preferably selected from the above-mentioned devices.

Particular preference is given to an organic electroluminescent device comprising anode, cathode and at least one emitting layer, characterized in that at least one organic layer comprising at least one compound of formula (I) is present in the device. Preference is given to an organic electroluminescent device comprising anode, cathode and at least one emitting layer, characterized in that at least one organic layer in the device, selected from hole-transporting and emitting layers, preferably selected from hole-transporting layers, comprises at least one compound of formula (I).

A hole-transporting layer is understood here to mean all layers disposed between anode and emitting layer, preferably hole injection layer, hole transport layer and electron blocking layer. A hole injection layer is understood here to mean a layer that directly adjoins the anode. A hole transport layer is understood here to mean a layer which is between the anode and emitting layer but does not directly adjoin the anode, and preferably does not directly adjoin the emitting layer either. An electron blocking layer is understood here to mean a layer which is between the anode and emitting layer and directly adjoins the emitting layer. An electron blocking layer preferably has a high-energy LUMO and hence prevents electrons from exiting from the emitting layer.

Apart from the cathode, anode and emitting layer, the electronic device may comprise further layers. These are selected, for example, from in each case one or more hole injection layers, hole transport layers, hole blocking layers, electron transport layers, electron injection layers, electron blocking layers, exciton blocking layers, interlayers, charge generation layers and/or organic or inorganic p/n junctions. However, it should be pointed out that not every one of these layers need necessarily be present and the choice of layers always depends on the compounds used and especially also on whether the device is a fluorescent or phosphorescent electroluminescent device.

The sequence of layers in the electronic device is preferably as follows:
—anode—
—hole injection layer—
—hole transport layer—
—optionally further hole transport layers—
—optionally electron blocking layer—
—emitting layer—
—optionally hole blocking layer—
—electron transport layer—
—electron injection layer—
—cathode—.

At the same time, it should be pointed out again that not all the layers mentioned need be present and/or that further layers may additionally be present.

The organic electroluminescent device of the invention may contain two or more emitting layers. More preferably, these emission layers have several emission maxima between 380 nm and 750 nm overall, such that the overall result is white emission; in other words, various emitting compounds which may fluoresce or phosphoresce and which emit blue, green, yellow, orange or red light are used in the emitting layers. Especially preferred are three-layer systems, i.e. systems having three emitting layers, wherein one of the three layers in each case shows blue emission, one of the three layers in each case shows green emission, and one of the three layers in each case shows orange or red emission. The compounds of the invention here are preferably present in a hole-transporting layer or in the emitting layer. It should be noted that, for the production of white light, rather than a plurality of colour-emitting emitter compounds, an emitter compound used individually which emits over a broad wavelength range may also be suitable.

It is preferable that the compound of the formula (I) is used as hole transport material. The emitting layer here may be a fluorescent emitting layer, or it may be a phosphorescent emitting layer. The emitting layer is preferably a blue-fluorescing layer or a green-phosphorescing layer.

When the device containing the compound of the formula (I) contains a phosphorescent emitting layer, it is preferable that this layer contains two or more, preferably exactly two, different matrix materials (mixed matrix system). Preferred embodiments of mixed matrix systems are described in detail further down.

If the compound of formula (I) is used as hole transport material in a hole transport layer, a hole injection layer or an electron blocking layer, the compound can be used as pure material, i.e. in a proportion of 100%, in the hole transport layer, or it can be used in combination with one or more further compounds.

In a preferred embodiment, a hole-transporting layer comprising the compound of the formula (I) additionally comprises one or more further hole-transporting compounds. These further hole-transporting compounds are preferably selected from triarylamine compounds, more preferably from monotriarylamine compounds. They are most preferably selected from the preferred embodiments of hole transport materials that are specified further down. In the preferred embodiment described, the compound of the formula (I) and the one or more further hole-transporting compounds are preferably each present in a proportion of at least 10%, more preferably each in a proportion of at least 20%.

In a preferred embodiment, a hole-transporting layer comprising the compound of the formula (I) additionally contains one or more p-dopants. p-Dopants used according to the present invention are preferably those organic electron acceptor compounds capable of oxidizing one or more of the other compounds in the mixture.

Particularly preferred as p-dopants are quinodimethane compounds, azaindenofluorenediones, azaphenalenes, azatriphenylenes, I₂, metal halides, preferably transition metal halides, metal oxides, preferably metal oxides comprising at least one transition metal or a metal from main group 3, and transition metal complexes, preferably complexes of Cu, Co, Ni, Pd and Pt with ligands containing at least one oxygen atom as binding site. Preference is further given to transition metal oxides as dopants, preferably oxides of rhenium, molybdenum and tungsten, more preferably Re₂O₇, MoO₃, WO₃ and ReO₃. Still further preference is given to complexes of bismuth in the (III) oxidation state, more particularly bismuth(III) complexes with electron-deficient ligands, more particularly carboxylate ligands.

The p-dopants are preferably in substantially homogeneous distribution in the p-doped layers. This can be achieved, for example, by co-evaporation of the p-dopant and the hole transport material matrix. The p-dopant is preferably present in a proportion of 1% to 10% in the p-doped layer.

Preferred p-dopants are furthermore the compounds which are explicitly disclosed in the table on p. 86-87 of WO2021/156323A1.

In a preferred embodiment, a hole injection layer that conforms to one of the following embodiments is present in the device: a) it contains a triarylamine and a p-dopant; or b) it contains a single electron-deficient material (electron acceptor). In a preferred embodiment of embodiment a), the triarylamine is a monotriarylamine, especially one of the preferred triarylamine derivatives mentioned further down. In a preferred embodiment of embodiment b), the electron-deficient material is a hexaazatriphenylene derivative as described in US 2007/0092755.

The compound of the formula (I) may be present in a hole injection layer, in a hole transport layer and/or in an electron blocking layer of the device. When the compound is present in a hole injection layer or in a hole transport layer, it has preferably been p-doped, meaning that it is in mixed form with a p-dopant, as described above, in the layer.

The compound of the formula (I) is preferably present in an electron blocking layer. In this case, it is preferably not p-doped. Further preferably, in this case, it is preferably in the form of a single compound in the layer without addition of a further compound.

In an alternative preferred embodiment, the compound of the formula (I) is used in an emitting layer as matrix material in combination with one or more emitting compounds, preferably phosphorescent emitting compounds. The phosphorescent emitting compounds here are preferably selected from red-phosphorescing and green-phosphorescing compounds.

The proportion of the matrix material in the emitting layer in this case is between 50.0% and 99.9% by volume, preferably between 80.0% and 99.5% by volume, and more preferably between 85.0% and 97.0% by volume.

Correspondingly, the proportion of the emitting compound is between 0.1% and 50.0% by volume, preferably between 0.5% and 20.0% by volume, and more preferably between 3.0% and 15.0% by volume.

An emitting layer of an organic electroluminescent device may also contain systems comprising a plurality of matrix materials (mixed matrix systems) and/or a plurality of emitting compounds. In this case too, the emitting compounds are generally those compounds having the smaller proportion in the system and the matrix materials are those compounds having the greater proportion in the system. In individual cases, however, the proportion of a single matrix material in the system may be less than the proportion of a single emitting compound.

It is preferable that the compounds of formula (I) are used as a component of mixed matrix systems, preferably for phosphorescent emitters. The mixed matrix systems preferably comprise two or three different matrix materials, more preferably two different matrix materials. Preferably, in this case, one of the two materials is a material having hole-transporting properties and the other material is a material having electron-transporting properties. It is further preferable when one of the materials is selected from compounds having a large energy differential between HOMO and LUMO (wide-bandgap materials). The compound of the formula (I) in a mixed matrix system is preferably the matrix material having hole-transporting properties. Correspondingly, when the compound of the formula (I) is used as matrix material for a phosphorescent emitter in the emitting layer of an OLED, a second matrix compound having electron-transporting properties is present in the emitting layer. The two different matrix materials may be present here in a ratio of 1:50 to 1:1, preferably 1:20 to 1:1, more preferably 1:10 to 1:1 and most preferably 1:4 to 1:1.

The desired electron-transporting and hole-transporting properties of the mixed matrix components may, however, also be combined mainly or entirely in a single mixed matrix component, in which case the further mixed matrix component(s) fulfil(s) other functions.

Preference is given to using the following material classes in the above-mentioned layers of the device:

### Phosphorescent Emitters

The term “phosphorescent emitters” typically encompasses compounds where the emission of light is effected through a spin-forbidden transition, for example a transition from an excited triplet state or a state having a higher spin quantum number, for example a quintet state.

Suitable phosphorescent emitters are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitters, compounds containing copper, molybdenum, tungsten, rhenium, ruthenium, osmium, rhodium, iridium, palladium, platinum, silver, gold or europium, especially compounds containing iridium, platinum or copper.

In the context of the present invention, all luminescent iridium, platinum or copper complexes are considered to be phosphorescent compounds.

In general, all phosphorescent complexes as used for phosphorescent OLEDs according to the prior art and as known to those skilled in the art in the field of organic electroluminescent devices are suitable for use in the devices of the invention. Further examples of suitable phosphorescent emitters are those shown in the table on p. 100-104 of WO2023/025971A2.

### Fluorescent Emitters

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9,10 position. Aromatic pyreneamines, pyrenediamines, chryseneamines and chrysenediamines are defined analogously, where the diarylamino groups are bonded to the pyrene preferably in the 1 position or 1,6 position. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units.

### Matrix Materials for Fluorescent Emitters

Preferred matrix materials for fluorescent emitters are selected from the classes of the oligoarylenes (e.g. 2,2′,7,7′-tetraphenylspirobifluorene), especially the oligoarylenes containing fused aromatic groups, the oligoarylenevinylenes, the polypodal metal complexes, the hole-conducting compounds, the electron-conducting compounds, especially ketones, phosphine oxides and sulfoxides; the atropisomers, the boronic acid derivatives or the benzanthracenes. Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another.

### Matrix Materials for Phosphorescent Emitters

Preferred matrix materials for phosphorescent emitters are, as well as the compounds of the formula (I), aromatic ketones, aromatic phosphine oxides or aromatic sulfoxides or sulfones, triarylamines, carbazole derivatives, e.g. CBP (N,N-biscarbazolylbiphenyl) or carbazole derivatives, indolocarbazole derivatives, indenocarbazole derivatives, azacarbazole derivatives, bipolar matrix materials, silanes, azaboroles or boronic esters, triazine derivatives, zinc complexes, diazasilole or tetraazasilole derivatives, diazaphosphole derivatives, bridged carbazole derivatives, triphenylene derivatives, or lactams.

### Electron-Transporting Materials

Suitable electron-transporting materials are, for example, the compounds disclosed in Y. Shirota et al., Chem. Rev. 2007, 107(4), 953-1010, or other materials used in these layers according to the prior art.

Materials used for the electron transport layer may be any materials that are used as electron transport materials in the electron transport layer according to the prior art. Especially suitable are aluminium complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives.

Preferred electron transport and electron injection materials are those shown in the table on p. 73-75 of WO2020/109434A1.

### Hole-Transporting Materials

Further compounds which, in addition to the compounds of the formula (I), are preferably used in hole-transporting layers of the OLEDs of the invention are indenofluoreneamine derivatives, amine derivatives, hexaazatriphenylene derivatives, amine derivatives with fused aromatic systems, monobenzoindenofluoreneamines, dibenzoindenofluoreneamines, spirobifluoreneamines, fluoreneamines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines, and 9,10-dihydroanthracene spiro compounds having diarylamino groups. Preferred hole-transporting compounds are those shown the table on p. 76-80 of WO2020/109434A1.

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the emission of light (OLED, 0-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

In a preferred embodiment, the electronic device is characterized in that one or more layers are coated by a sublimation process. In this case, the materials are applied by vapour deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

Preference is likewise given to an electronic device, characterized in that one or more layers are coated by the OVPD (organic vapour phase deposition) method or with the aid of a carrier gas sublimation. In this case, the materials are applied at a pressure between 10⁻⁵ mbar and 1 bar. A special case of this method is the OVJP (organic vapour jet printing) method, in which the materials are applied directly by a nozzle and thus structured (for example M. S. Arnold et al., Appl. Phys. Lett. 2008, 92, 053301).

Preference is additionally given to an electronic device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds of formula (I) are needed. High solubility can be achieved by suitable substitution of the compounds.

It is further preferable that an electronic device of the invention is produced by applying one or more layers from solution and one or more layers by a sublimation method.

After application of the layers, according to the use, the device is structured, contact-connected and finally sealed, in order to rule out damaging effects of water and air.

According to the invention, the electronic devices comprising one or more compounds of formula (I) can be used in displays, as light sources in lighting applications and as light sources in medical and/or cosmetic applications.

### EXAMPLES

### A) Synthesis Examples

### Synthesis of Intermediate Int-1.1

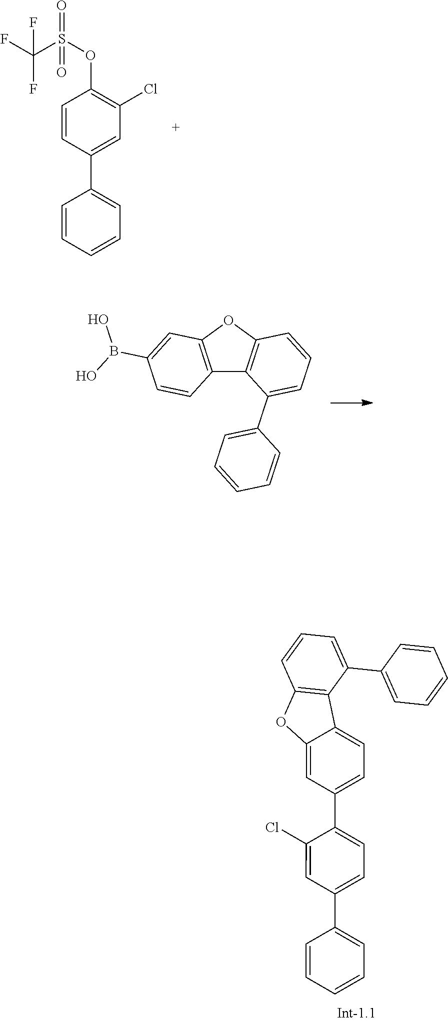 [SMILES: O=S(=O)(Oc1ccc(-c2ccccc2)cc1Cl)C(F)(F)F ; OB(O)c1ccc2c(c1)oc1cccc(-c3ccccc3)c12 ; Clc1cc(-c2ccccc2)ccc1-c1ccc2c(c1)oc1cccc(-c3ccccc3)c12]

36.0 g (100 mmol) 3-chloro-[1,1′-biphenyl]-4-yl trifluoromethanesulfonate (CAS175153-76-1), 43.3 g (150.4 mmol) (9-Phenyldibenzo[b,d]furan-3-yl)boronic acid (CAS 2266590-81-0, CN114621252) and 31.9 g (300.8 mmol) disodium carbonate are dissolved in 1000 ml dioxane/toluene/water (1:2:1). 2.3 g (2.0 mmol) Tetrakis(triphenylphosphin)-palladium(0) are added and the mixture is refluxed overnight. After full conversion, the reaction mixture is allowed to come to room temperature, 500 ml brine is added, and the two phases are separated. The organic phase is reduced under reduced pressure and the residue is purified by column chromatography (heptane/toluene 5:1; SiO₂) and further purified by crystallization out of heptane.

Yield: 31.8 g (73.8 mmol, 74%)

Following compounds can be synthesized in similar manner:

| Int- | educt 1 | educt 2 | product |
|---|---|---|---|
| 1.2 | 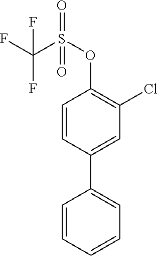 [SMILES: O=S(=O)(Oc1ccc(-c2ccccc2)cc1Cl)C(F)(F)F] CAS175153-76-1 | 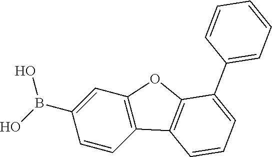 [SMILES: OB(O)c1ccc2c(c1)oc1c(-c3ccccc3)cccc12] CAS 2266590-93-4 | 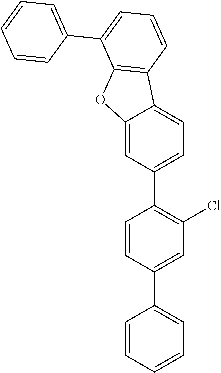 [SMILES: Clc1cc(-c2ccccc2)ccc1-c1ccc2c(c1)oc1c(-c3ccccc3)cccc12] |
| 1.3 | 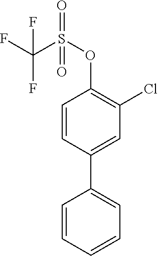 [SMILES: O=S(=O)(Oc1ccc(-c2ccccc2)cc1Cl)C(F)(F)F] CAS175153-76-1 | 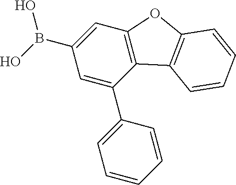 [SMILES: OB(O)c1cc(-c2ccccc2)c2c(c1)oc1ccccc12] CAS 2869873-86-7 | 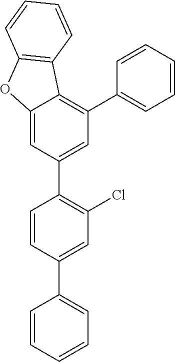 [SMILES: Clc1cc(-c2ccccc2)ccc1-c1cc(-c2ccccc2)c2c(c1)oc1ccccc12] |
| 1.4 | 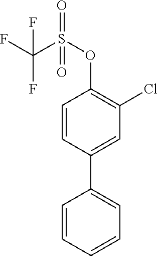 [SMILES: O=S(=O)(Oc1ccc(-c2ccccc2)cc1Cl)C(F)(F)F] CAS175153-76-1 | 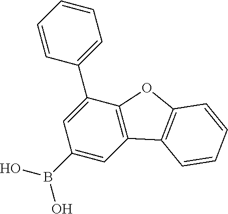 [SMILES: OB(O)c1cc(-c2ccccc2)c2oc3ccccc3c2c1] CAS 2786816-88-2 | 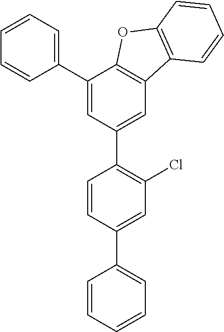 [SMILES: Clc1cc(-c2ccccc2)ccc1-c1cc(-c2ccccc2)c2oc3ccccc3c2c1] |
| 1.5 | 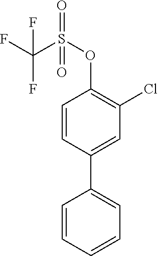 [SMILES: O=S(=O)(Oc1ccc(-c2ccccc2)cc1Cl)C(F)(F)F] CAS175153-76-1 | 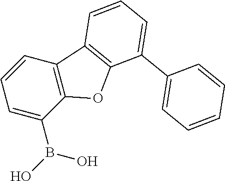 [SMILES: OB(O)c1cccc2c1oc1c(-c3ccccc3)cccc12] CAS 1010068-85-5 | 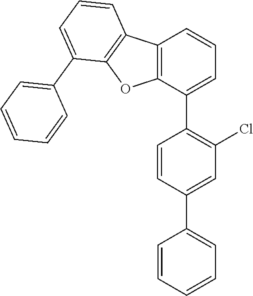 [SMILES: Clc1cc(-c2ccccc2)ccc1-c1cccc2c1oc1c(-c3ccccc3)cccc12] |
| 1.6 | 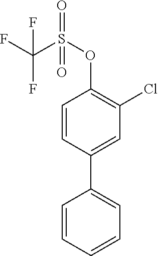 [SMILES: O=S(=O)(Oc1ccc(-c2ccccc2)cc1Cl)C(F)(F)F] CAS175153-76-1 | 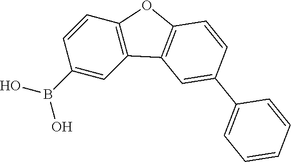 [SMILES: OB(O)c1ccc2oc3ccc(-c4ccccc4)cc3c2c1] CAS 854952-58-2 | 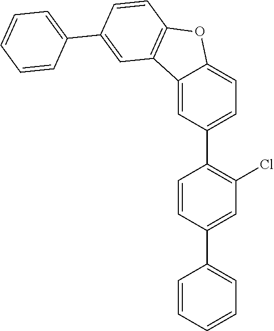 [SMILES: Clc1cc(-c2ccccc2)ccc1-c1ccc2oc3ccc(-c4ccccc4)cc3c2c1] |
| 1.7 | 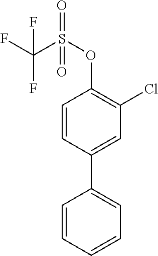 [SMILES: O=S(=O)(Oc1ccc(-c2ccccc2)cc1Cl)C(F)(F)F] CAS175153-76-1 | 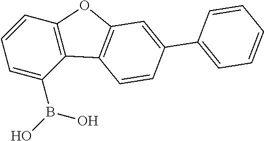 [SMILES: OB(O)c1cccc2oc3cc(-c4ccccc4)ccc3c12] CAS1821233-94-6 | 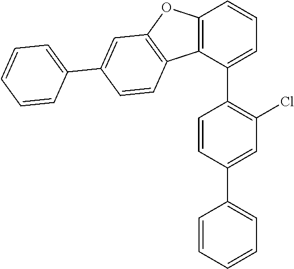 [SMILES: Clc1cc(-c2ccccc2)ccc1-c1cccc2oc3cc(-c4ccccc4)ccc3c12] |
| 1.8 | 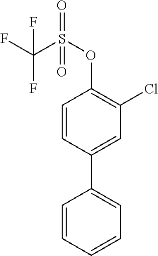 [SMILES: O=S(=O)(Oc1ccc(-c2ccccc2)cc1Cl)C(F)(F)F] CAS175153-76-1 | 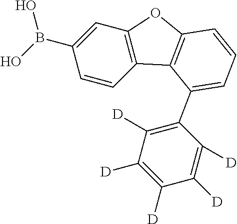 [SMILES: [2H]c1c([2H])c([2H])c(-c2cccc3oc4cc(B(O)O)ccc4c23)c([2H])c1[2H]] CAS 2266619-18-3 | 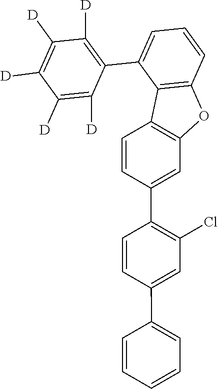 [SMILES: [2H]c1c([2H])c([2H])c(-c2cccc3oc4cc(-c5ccc(-c6ccccc6)cc5Cl)ccc4c23)c([2H])c1[2H]] |
| 1.9 | 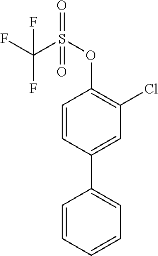 [SMILES: O=S(=O)(Oc1ccc(-c2ccccc2)cc1Cl)C(F)(F)F] CAS175153-76-1 | 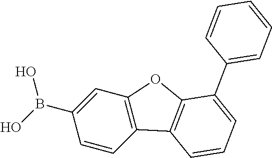 [SMILES: OB(O)c1ccc2c(c1)oc1c(-c3ccccc3)cccc12] CAS 2266590-93-4 | 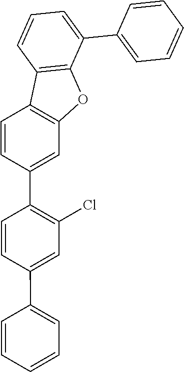 [SMILES: Clc1cc(-c2ccccc2)ccc1-c1ccc2c(c1)oc1c(-c3ccccc3)cccc12] |
| 1.10 | 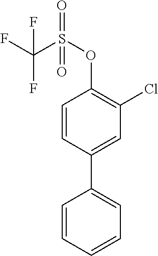 [SMILES: O=S(=O)(Oc1ccc(-c2ccccc2)cc1Cl)C(F)(F)F] CAS175153-76-1 | 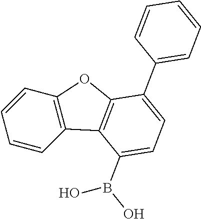 [SMILES: OB(O)c1ccc(-c2ccccc2)c2oc3ccccc3c12] CAS 1821234-09-6 | 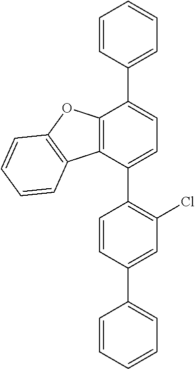 [SMILES: Clc1cc(-c2ccccc2)ccc1-c1ccc(-c2ccccc2)c2oc3ccccc3c12] |
| 1.11 | 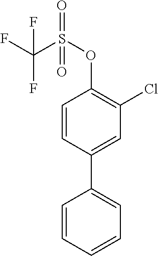 [SMILES: O=S(=O)(Oc1ccc(-c2ccccc2)cc1Cl)C(F)(F)F] CAS175153-76-1 | 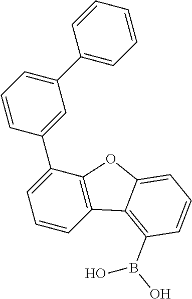 [SMILES: OB(O)c1cccc2oc3c(-c4cccc(-c5ccccc5)c4)cccc3c12] CAS 1822310-24-6 | 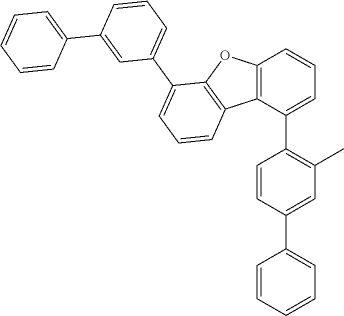 [SMILES: Cc1cc(-c2ccccc2)ccc1-c1cccc2oc3c(-c4cccc(-c5ccccc5)c4)cccc3c12] |

### Synthesis of Compound 1

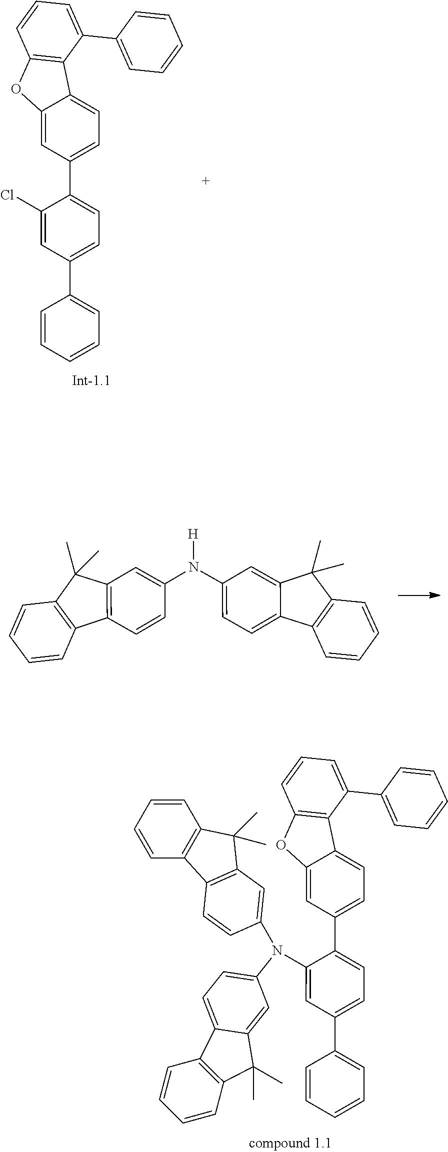 [SMILES: Clc1cc(-c2ccccc2)ccc1-c1ccc2c(c1)oc1cccc(-c3ccccc3)c12 ; CC1(C)c2ccccc2-c2ccc(Nc3ccc4c(c3)C(C)(C)c3ccccc3-4)cc21 ; CC1(C)c2ccccc2-c2ccc(N(c3ccc4c(c3)C(C)(C)c3ccccc3-4)c3cc(-c4ccccc4)ccc3-c3ccc4c(c3)oc3cccc(-c5ccccc5)c34)cc21]

29.8 g (69.1 mmol) Int-1.1 and 27.7 g (69.1 mmol) Bis(9,9-dimethyl-9-H-fluoren-2-yl)amine are dissolved in 600 ml toluene. 0.98 g (61.4 mmol) Bis(di-tert-butyl(4-dimethylaminophenyl)phosphine) dichloropalladium(II) and 31.0 mL sodium tert-pentoxide solution (103.7 mmol; 40% in toluene) are added. The mixture is stirred at 60° C. for 18 hours. After full conversion, the reaction mixture is allowed to come to room temperature and 500 ml water are added, and the two phases are separated. The organic phase is washed two times with 400 ml water and two times with 300 ml brine. The organic phase is reduced under reduced pressure and the residue is crystallized out of ethyl acetate. The solid is further purified by crystallization out of toluene/heptane until a HPLC purity of >99.9% and the remaining solvents are removed by sublimation (310° C.; <10⁻⁵ bar).

Yield: 32.7 g (41.1 mmol, 60.0%)

Following compounds can be synthesized in similar manner:

| Compound- | Int- | Starting material B | Product |
|---|---|---|---|
| 2 | 1.2 | 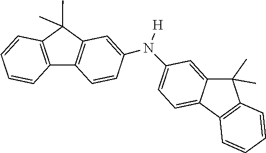 [SMILES: CC1(C)c2ccccc2-c2ccc(Nc3ccc4c(c3)C(C)(C)c3ccccc3-4)cc21] CAS 500717-23-7 | 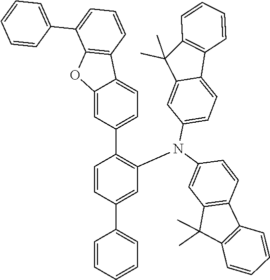 [SMILES: CC1(C)c2ccccc2-c2ccc(N(c3ccc4c(c3)C(C)(C)c3ccccc3-4)c3cc(-c4ccccc4)ccc3-c3ccc4c(c3)oc3c(-c5ccccc5)cccc34)cc21] |
| 3 | 1.3 | 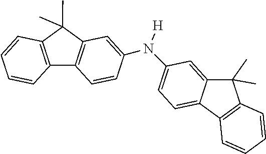 [SMILES: CC1(C)c2ccccc2-c2ccc(Nc3ccc4c(c3)C(C)(C)c3ccccc3-4)cc21] CAS 500717-23-7 | 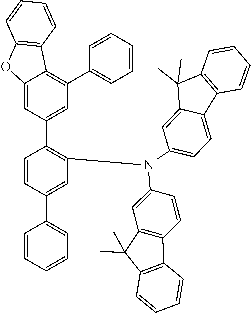 [SMILES: CC1(C)c2ccccc2-c2ccc(N(c3ccc4c(c3)C(C)(C)c3ccccc3-4)c3cc(-c4ccccc4)ccc3-c3cc(-c4ccccc4)c4c(c3)oc3ccccc34)cc21] |
| 4 | 1.4 | 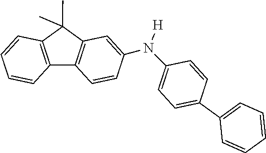 [SMILES: CC1(C)c2ccccc2-c2ccc(Nc3ccc(-c4ccccc4)cc3)cc21] CAS 897671-69-1 | 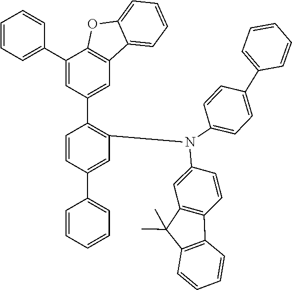 [SMILES: CC1(C)c2ccccc2-c2ccc(N(c3ccc(-c4ccccc4)cc3)c3cc(-c4ccccc4)ccc3-c3cc(-c4ccccc4)c4oc5ccccc5c4c3)cc21] |
| 5 | 1.5 | 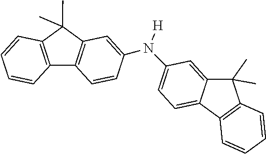 [SMILES: CC1(C)c2ccccc2-c2ccc(Nc3ccc4c(c3)C(C)(C)c3ccccc3-4)cc21] CAS 500717-23-7 | 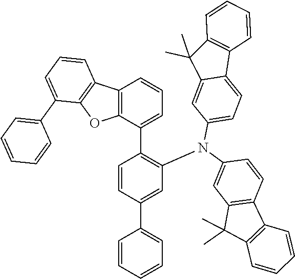 [SMILES: CC1(C)c2ccccc2-c2ccc(N(c3ccc4c(c3)C(C)(C)c3ccccc3-4)c3cc(-c4ccccc4)ccc3-c3cccc4c3oc3c(-c5ccccc5)cccc34)cc21] |
| 6 | 1.6 | 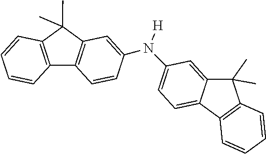 [SMILES: CC1(C)c2ccccc2-c2ccc(Nc3ccc4c(c3)C(C)(C)c3ccccc3-4)cc21] CAS 500717-23-7 | 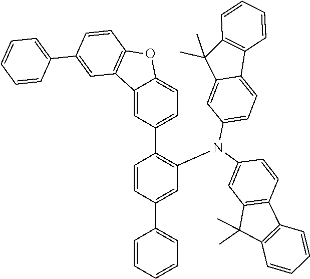 [SMILES: CC1(C)c2ccccc2-c2ccc(N(c3ccc4c(c3)C(C)(C)c3ccccc3-4)c3cc(-c4ccccc4)ccc3-c3ccc4oc5ccc(-c6ccccc6)cc5c4c3)cc21] |
| 7 | 1.2 | 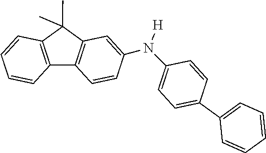 [SMILES: CC1(C)c2ccccc2-c2ccc(Nc3ccc(-c4ccccc4)cc3)cc21] CAS 897671-69-1 | 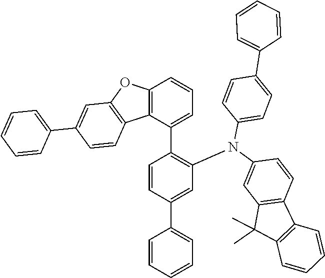 [SMILES: CC1(C)c2ccccc2-c2ccc(N(c3ccc(-c4ccccc4)cc3)c3cc(-c4ccccc4)ccc3-c3cccc4oc5cc(-c6ccccc6)ccc5c34)cc21] |
| 8 | 1.8 | 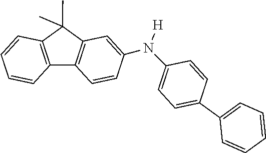 [SMILES: CC1(C)c2ccccc2-c2ccc(Nc3ccc(-c4ccccc4)cc3)cc21] CAS 897671-69-1 | 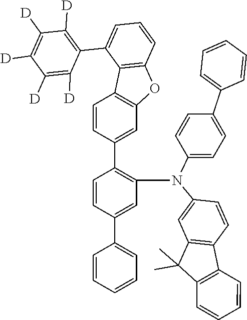 [SMILES: [2H]c1c([2H])c([2H])c(-c2cccc3oc4cc(-c5ccc(-c6ccccc6)cc5N(c5ccc(-c6ccccc6)cc5)c5ccc6c(c5)C(C)(C)c5ccccc5-6)ccc4c23)c([2H])c1[2H]] |
| 9 | 1.1 | 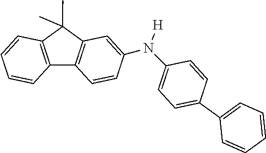 [SMILES: CC1(C)c2ccccc2-c2ccc(Nc3ccc(-c4ccccc4)cc3)cc21] CAS 102113-98-4 | 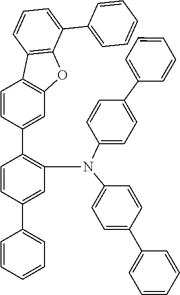 [SMILES: c1ccc(-c2ccc(N(c3ccc(-c4ccccc4)cc3)c3cc(-c4ccccc4)ccc3-c3ccc4c(c3)oc3c(-c5ccccc5)cccc34)cc2)cc1] |
| 10 | 1.1 | 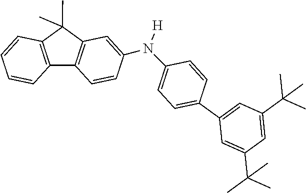 [SMILES: CC(C)(C)c1cc(-c2ccc(Nc3ccc4c(c3)C(C)(C)c3ccccc3-4)cc2)cc(C(C)(C)C)c1] CAS 2572529-05-4 | 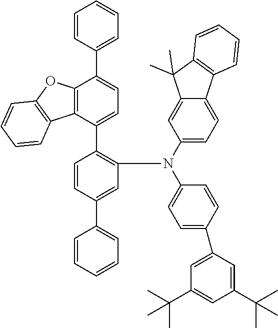 [SMILES: CC(C)(C)c1cc(-c2ccc(N(c3ccc4c(c3)C(C)(C)c3ccccc3-4)c3cc(-c4ccccc4)ccc3-c3ccc(-c4ccccc4)c4oc5ccccc5c34)cc2)cc(C(C)(C)C)c1] |
| 11 | 1.7 | 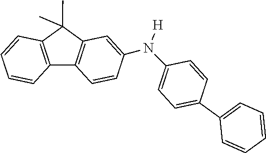 [SMILES: CC1(C)c2ccccc2-c2ccc(Nc3ccc(-c4ccccc4)cc3)cc21] CAS 897671-69-1 | 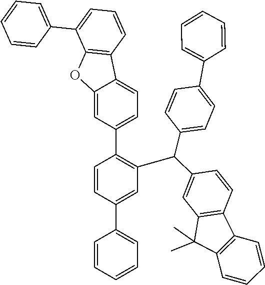 [SMILES: CC1(C)c2ccccc2-c2ccc(C(c3ccc(-c4ccccc4)cc3)c3cc(-c4ccccc4)ccc3-c3ccc4c(c3)oc3c(-c5ccccc5)cccc34)cc21] |
| 12 | 1.8 | 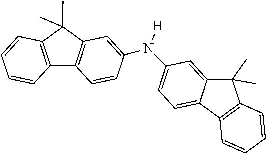 [SMILES: CC1(C)c2ccccc2-c2ccc(Nc3ccc4c(c3)C(C)(C)c3ccccc3-4)cc21] CAS 500717-23-7 | 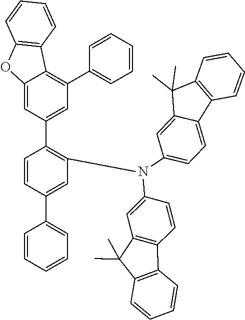 [SMILES: CC1(C)c2ccccc2-c2ccc(N(c3ccc4c(c3)C(C)(C)c3ccccc3-4)c3cc(-c4ccccc4)ccc3-c3cc(-c4ccccc4)c4c(c3)oc3ccccc34)cc21] |
| 13 | 1.11 | 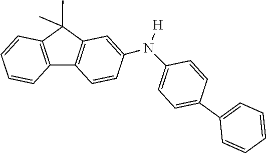 [SMILES: CC1(C)c2ccccc2-c2ccc(Nc3ccc(-c4ccccc4)cc3)cc21] CAS 897671-69-1 | 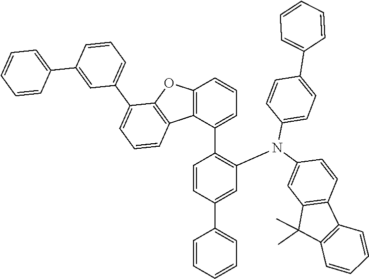 [SMILES: CC1(C)c2ccccc2-c2ccc(N(c3ccc(-c4ccccc4)cc3)c3cc(-c4ccccc4)ccc3-c3cccc4oc5c(-c6cccc(-c7ccccc7)c6)cccc5c34)cc21] |
| 14 | 1.10 | 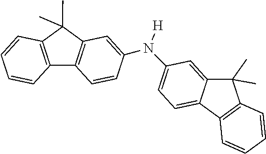 [SMILES: CC1(C)c2ccccc2-c2ccc(Nc3ccc4c(c3)C(C)(C)c3ccccc3-4)cc21] CAS 500717-23-7 | 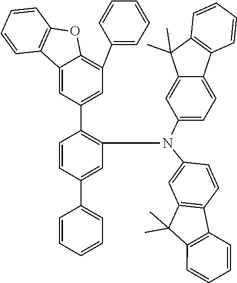 [SMILES: CC1(C)c2ccccc2-c2ccc(N(c3ccc4c(c3)C(C)(C)c3ccccc3-4)c3cc(-c4ccccc4)ccc3-c3cc(-c4ccccc4)c4oc5ccccc5c4c3)cc21] |
| 15 | 1.1 | 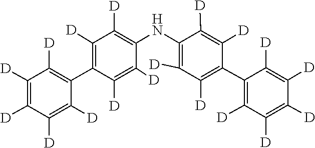 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] CAS 1879963-55-9 | 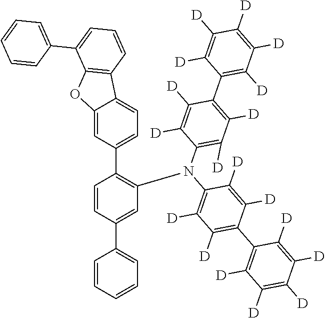 [SMILES: [2H]c1c([2H])c([2H])c(-c2c([2H])c([2H])c(N(c3cc(-c4ccccc4)ccc3-c3ccc4c(c3)oc3c(-c5ccccc5)cccc34)c3c([2H])c([2H])c(-c4c([2H])c([2H])c([2H])c([2H])c4[2H])c([2H])c3[2H])c([2H])c2[2H])c([2H])c1[2H]] |

### B) Device Examples

### 1) General Production Process for the OLEDs and Characterization of the OLEDs

Glass plaques which have been coated with structured ITO (indium tin oxide) in a thickness of 50 nm are the substrates to which the OLEDs are applied.

The OLEDs basically have the following layer structure: substrate/hole injection layer (HIL)/hole transport layer (HTL)/electron blocker layer (EBL)/emission layer (EML)/electron transport layer, optionally with second layer (HBL, ETL1/ETL)/electron injection layer (EIL) and finally a cathode. The cathode is formed by an aluminium layer of thickness 100 nm. The exact structure of the OLEDs can be found in the tables which follow. The materials used for production of the OLEDs are shown in a table below. The compound “EBM” is a spirobifluorenyl amine derivate. The compound “HTM” is a bis-2-fluorenyl amine derivate.

All materials are applied by thermal vapour deposition in a vacuum chamber. In this case, the emission layer consists of at least one matrix material (host material) and an emitting dopant which is added to the matrix material(s) in a particular proportion by volume by co-evaporation. Details given in such a form as H:SEB (95%:5%) mean here that the material H is present in the layer in a proportion by volume of 95% and SEB in a proportion of 5%. In an analogous manner, the electron transport layer and the hole injection layer also consist of a mixture of two materials. The structures of the materials that are used in the OLEDs are shown in Table 1.

The OLEDs are characterized in a standard manner. For this purpose, the electroluminescence spectra, the external quantum efficiency (EQE, measured in %) as a function of the luminance, calculated from current-voltage-luminance characteristics assuming Lambertian radiation characteristics, and the lifetime are determined. The parameter EQE @10 mA/cm² refers to the external quantum efficiency which is attained at 10 mA/cm². The parameter U @10 mA/cm² refers to the operating voltage at 10 mA/cm². The lifetime LT is defined as the time after which the luminance drops from the starting luminance to a certain proportion in the course of operation with constant current density. An LT90 figure means here that the lifetime reported corresponds to the time after which the luminance has dropped to 90% of its starting value. The figure @1000 cd/m² or 15000 cd/m² means here that the lifetime in question is measured at constant luminance of 1000 cd/m² or 15000 cd/m².

TABLE 1
Structures of the compounds

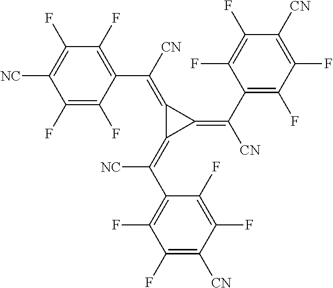 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

p-dopant

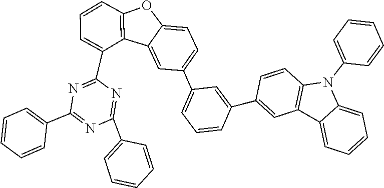 [SMILES: c1ccc(-c2nc(-c3ccccc3)nc(-c3cccc4oc5ccc(-c6cccc(-c7ccc8c(c7)c7ccccc7n8-c7ccccc7)c6)cc5c34)n2)cc1]

TMM-1

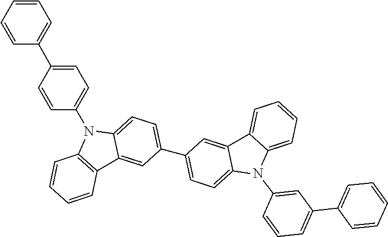 [SMILES: c1ccc(-c2ccc(-n3c4ccccc4c4cc(-c5ccc6c(c5)c5ccccc5n6-c5cccc(-c6ccccc6)c5)ccc43)cc2)cc1]

TMM-2

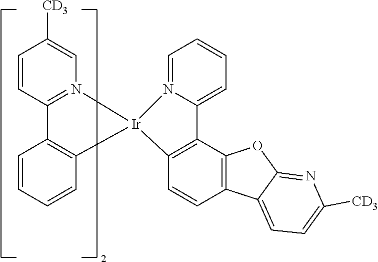

TEG

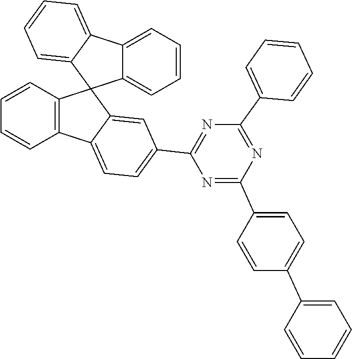 [SMILES: c1ccc(-c2ccc(-c3nc(-c4ccccc4)nc(-c4ccc5c(c4)C4(c6ccccc6-c6ccccc64)c4ccccc4-5)n3)cc2)cc1]

ETM

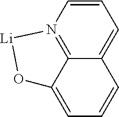

LiQ

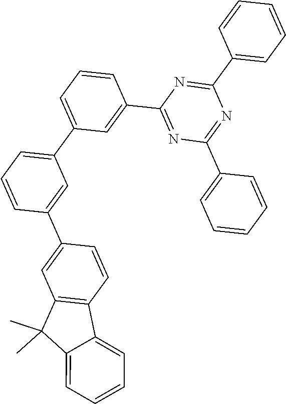 [SMILES: CC1(C)c2ccccc2-c2ccc(-c3cccc(-c4cccc(-c5nc(-c6ccccc6)nc(-c6ccccc6)n5)c4)c3)cc21]

HBM

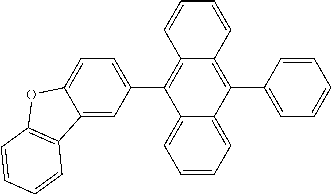 [SMILES: c1ccc(-c2c3ccccc3c(-c3ccc4oc5ccccc5c4c3)c3ccccc23)cc1]

H

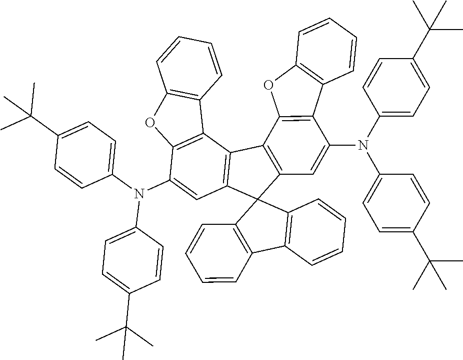 [SMILES: CC(C)(C)c1ccc(N(c2ccc(C(C)(C)C)cc2)c2cc3c(c4c2oc2ccccc24)-c2c(cc(N(c4ccc(C(C)(C)C)cc4)c4ccc(C(C)(C)C)cc4)c4c2oc2ccccc24)C32c3ccccc3-c3ccccc32)cc1]

SEB

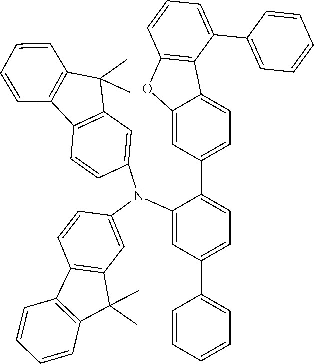 [SMILES: CC1(C)c2ccccc2-c2ccc(N(c3ccc4c(c3)C(C)(C)c3ccccc3-4)c3cc(-c4ccccc4)ccc3-c3ccc4c(c3)oc3cccc(-c5ccccc5)c34)cc21]

Inv-1

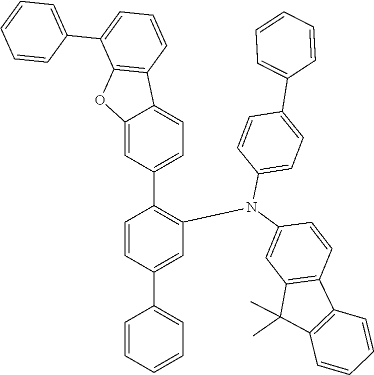 [SMILES: CC1(C)c2ccccc2-c2ccc(N(c3ccc(-c4ccccc4)cc3)c3cc(-c4ccccc4)ccc3-c3ccc4c(c3)oc3c(-c5ccccc5)cccc34)cc21]

Inv-2

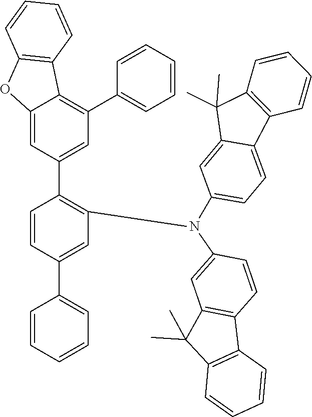 [SMILES: CC1(C)c2ccccc2-c2ccc(N(c3ccc4c(c3)C(C)(C)c3ccccc3-4)c3cc(-c4ccccc4)ccc3-c3cc(-c4ccccc4)c4c(c3)oc3ccccc34)cc21]

Inv-3

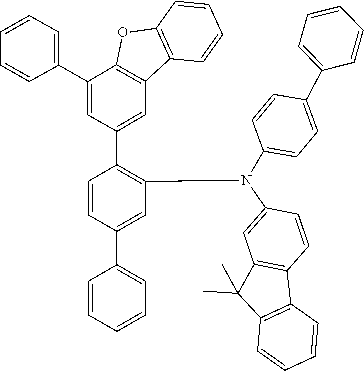 [SMILES: CC1(C)c2ccccc2-c2ccc(N(c3ccc(-c4ccccc4)cc3)c3cc(-c4ccccc4)ccc3-c3cc(-c4ccccc4)c4oc5ccccc5c4c3)cc21]

Inv-4

The compounds Inv-1 to Inv-4 are used in OLEDs as detailed below, showing good performance:

### 1) Inventive OLEDs Containing a Compound of the Formula (1) in the EBL of Green-Phosphorescing OLEDs

TABLE 2
Structure of the OLEDs

| | HIL | HTL1 | EBL | EML | HBL | ETL1 | EIL |
|---|---|---|---|---|---|---|---|
| | Thickness/ | Thickness/ | Thickness/ | Thickness/ | Thickness/ | Thickness/ | Thickness/ |
| Ex. | nm | nm | nm | nm | nm | nm | nm |
| 1 | HTM: | HTM | Inv-1 | TMM-1 (32%): | HBM | ETM:LiQ | LiQ |
| | p-Dotand | 50 nm | 30 nm | TMM-2 | 5 nm | (50:50%) | 1 nm |
| | (3%) | | | (60%): TEG (8%) | | 30 nm | |
| | 10 nm | | | 35 nm | | | |
| 2 | HTM: | HTM | Inv-2 | TMM-1 (32%): | HBM | ETM:LiQ | LiQ |
| | p-Dotand | 50 nm | 30 nm | TMM-2 | 5 nm | (50:50%) | 1 nm |
| | (3%) | | | (60%): TEG (8%) | | 30 nm | |
| | 10 nm | | | 35 nm | | | |
| 3 | HTM: | HTM | Inv-3 | TMM-1 (32%): | HBM | ETM:LiQ | LiQ |
| | p-Dotand | 50 nm | 30 nm | TMM-2 | 5 nm | (50:50%) | 1 nm |
| | (3%) | | | (60%): TEG (8%) | | 30 nm | |
| | 10 nm | | | 35 nm | | | |
| 4 | HTM: | HTM | Inv-4 | TMM-1 (32%): | HBM | ETM:LiQ | LiQ |
| | p-Dotand | 50 nm | 30 nm | TMM-2 | 5 nm | (50:50%) | 1 nm |
| | (3%) | | | (60%): TEG (8%) | | 30 nm | |
| | 10 nm | | | 35 nm | | | |

In the device setup shown above, the compounds Inv-1 to Inv-4 according to the invention give very good efficiency and lifetime:

TABLE 3
Results of the OLED device

| | U @ 10 mA/cm² | EQE @ 10 mA/cm² | LT95 @ 15000 cd/m² |
|---|---|---|---|
| Ex. | (V) | (%) | (h) |
| 1 | 3.4 | 23.2 | 514 |
| 2 | 4.1 | 23.2 | 496 |
| 3 | 3.4 | 23.2 | 479 |
| 4 | 4.3 | 23.0 | 483 |

### 2) Inventive OLEDs Containing a Compound of the Formula (I) in the HIL and HTL of Blue-Fluorescing OLEDs

TABLE 4
Structure of the OLEDs

| | HIL | HTL | EBL | EML | ETL | EIL |
|---|---|---|---|---|---|---|
| | Thickness/ | Thickness/ | Thickness/ | Thickness/ | Thickness/ | Thickness/ |
| Ex. | nm | nm | nm | nm | nm | nm |
| 5 | INV-1: p-dopant | Inv-1 | EBM | H: SEB(3%) | ETM: LiQ(50%) | LiQ |
| | (3%) | 180 nm | 5 nm | 20 nm | 30 nm | 1 nm |
| | 10 nm | | | | | |
| 6 | INV-3: p-dopant | Inv-3 | EBM | H: SEB(3%) | ETM: LiQ(50%) | LiQ |
| | (3%) | 180 nm | 5 nm | 20 nm | 30 nm | 1 nm |
| | 10 nm | | | | | |
| 7 | HTM: | HTM | Inv-2 | H: SEB(3%) | ETM: LiQ(50%) | LiQ |
| | p-Dotand (3%) | 180 nm | | 20 nm | 30 nm | 1 nm |
| | 10 nm | | | | | |
| 8 | HTM: | HTM | Inv-4 | H: SEB(3%) | ETM: LiQ(50%) | LiQ |
| | p-Dotand (3%) | 180 nm | | 20 nm | 30 nm | 1 nm |
| | 10 nm | | | | | |

In the device setup shown above, the compounds Inv-1 to Inv-4 according to the invention give very good efficiency and lifetime:

TABLE 5
Results of the OLED device

| | U @ 10 mA/cm² | EQE @ 10 mA/cm² | LT90 @ 1000 cd/m² |
|---|---|---|---|
| Ex. | (V) | (%) | (h) |
| 5 | 4.0 | 9.6 | 500 |
| 6 | 3.9 | 10.8 | 560 |
| 7 | 3.7 | 9.6 | 607 |
| 8 | 3.7 | 9.6 | 542 |

1. A compound according to the following formula (I) 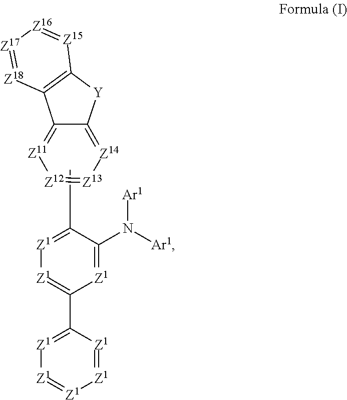
wherein the following applies to the variables present in the formula:
Z¹ is, identically or differently at each occurrence, selected from CR¹ and N;
Z¹¹, Z¹², Z¹³, Z¹⁴ is each, identically or differently, selected from CR¹, CR¹¹ and N, if the bond drawn into the ring comprising them is not attached to it, and Z¹¹, Z¹², Z¹³, Z¹⁴ is C, if the bond drawn into the ring comprising them is attached to it;
Z¹⁵, Z¹⁶, Z¹⁷, Z¹⁸ is, identically or differently at each occurrence, selected from CR¹, CR¹¹ and N;
Y is selected from O and S;
Ar¹ is selected, identically or differently at each occurrence, from phenyl, biphenyl, terphenyl, and quaterphenyl, where each of phenyl, biphenyl, terphenyl and quaterphenyl is substituted with radicals R²¹, and fluorenyl, which is attached in one of its positions 1 to 4, and which is substituted with radicals R²;
R¹ is selected, identically or differently on each occurrence, from H, D, F, C(═O)R³, CN, Si(R³)₃, N(R³)₂, P(═O)(R³)₂, OR³, S(═O)R³, S(═O)₂R³, straight-chain alkyl or alkoxy groups having 1 to 20 C atoms, branched or cyclic alkyl or alkoxy groups having 3 to 20 C atoms, alkenyl or alkynyl groups having 2 to 20 C atoms, aromatic ring systems having 6 to 40 aromatic ring atoms, and heteroaromatic ring systems having 5 to 40 aromatic ring atoms; where two or more radicals R¹ may be connected to each other to form a ring; where the said alkyl, alkoxy, alkenyl and alkynyl groups and the said aromatic ring systems and heteroaromatic ring systems are substituted by radicals R³, and where one or more CH₂ groups in the said alkyl, alkoxy, alkenyl and alkynyl groups may in each case be replaced by —R³C═CR³—, —C≡C—, Si(R³)₂, C═O, C═NR³, —C(═O)O—, —C(═O)NR³—, NR³, P(═O)(R³), —O—, —S—, SO or SO₂;
R¹¹ is selected, identically or differently on each occurrence, from aromatic ring systems having 6 to 24 aromatic ring atoms and heteroaromatic ring systems having 5 to 24 aromatic ring atoms; where the said said aromatic ring systems and heteroaromatic ring systems are each substituted by radicals R³;
R² is selected, identically or differently on each occurrence, from H, D, F, C(═O)R³, CN, Si(R³)₃, N(R³)₂, P(═O)(R³)₂, OR³, S(═O)R³, S(═O)₂R³, straight-chain alkyl or alkoxy groups having 1 to 20 C atoms, branched or cyclic alkyl or alkoxy groups having 3 to 20 C atoms, alkenyl or alkynyl groups having 2 to 20 C atoms, aromatic ring systems having 6 to 40 aromatic ring atoms, and heteroaromatic ring systems having 5 to 40 aromatic ring atoms; where two or more radicals R² may be connected to each other to form a ring; where the said alkyl, alkoxy, alkenyl and alkynyl groups and the said aromatic ring systems and heteroaromatic ring systems are substituted by radicals R³, and where one or more CH₂ groups in the said alkyl, alkoxy, alkenyl and alkynyl groups may in each case be replaced by —R³C═CR³—, —C≡C—, Si(R³)₂, C═O, C═NR³, —C(═O)O—, —C(═O)NR³—, NR³, P(═O)(R³), —O—, —S—, SO or SO₂;
R²¹ is selected, identically or differently on each occurrence, from H, D, F, C(═O)R³, CN, Si(R³)₃, N(R³)₂, P(═O)(R³)₂, OR³, S(═O)R³, S(═O)₂R³, straight-chain alkyl or alkoxy groups having 1 to 20 C atoms, branched or cyclic alkyl or alkoxy groups having 3 to 20 C atoms, alkenyl or alkynyl groups having 2 to 20 C atoms, where two or more radicals R²¹ may be connected to each other to form a ring; where the said alkyl, alkoxy, alkenyl and alkynyl groups are substituted by radicals R³, and where one or more CH₂ groups in the said alkyl, alkoxy, alkenyl and alkynyl groups may in each case be replaced by —R³C═CR³—, —C≡C—, Si(R³)₂, C═O, C═NR³, —C(═O)O—, —C(═O)NR³—, NR³, P(═O)(R³), —O—, —S—, SO or SO₂;
R³ is selected, identically or differently on each occurrence, from H, D, F, Cl, Br, I, C(═O)R⁴, CN, Si(R⁴)₃, N(R⁴)₂, P(═O)(R⁴)₂, OR⁴, S(═O)R⁴, S(═O)₂R⁴, straight-chain alkyl or alkoxy groups having 1 to 20 C atoms, branched or cyclic alkyl or alkoxy groups having 3 to 20 C atoms, alkenyl or alkynyl groups having 2 to 20 C atoms, aromatic ring systems having 6 to 40 aromatic ring atoms, and heteroaromatic ring systems having 5 to 40 aromatic ring atoms; where two or more radicals R³ may be connected to each other to form a ring; where the said alkyl, alkoxy, alkenyl and alkynyl groups and the said aromatic ring systems and heteroaromatic ring systems are substituted by radicals R⁴, and where one or more CH₂ groups in the said alkyl, alkoxy, alkenyl and alkynyl groups may in each case be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂;
R⁴ is selected, identically or differently on each occurrence, from H, D, F, Cl, Br, I, CN, alkyl groups having 1 to 20 C atoms, aromatic ring systems having 6 to 40 C atoms, and heteroaromatic ring systems having 5 to 40 aromatic ring atoms; where two or more radicals R⁴ may be connected to each other to form a ring; and where the said alkyl groups, aromatic ring systems and heteroaromatic ring systems may be substituted by one or more radicals selected from F and CN;
wherein the bond drawn into the ring comprising Z¹¹, Z¹², Z¹³ and Z¹⁴ is attached to one of Z¹¹, Z¹², Z¹³, and Z¹⁴, and
wherein at least one of Z¹¹, Z¹², Z¹³, Z¹⁴, Z¹⁵, Z¹⁶, Z¹⁷ and Z¹⁸ is CR¹¹.

2. The compound according to claim 1, characterized in that it conforms to one of the following formulae:

| | |
|---|---|
| 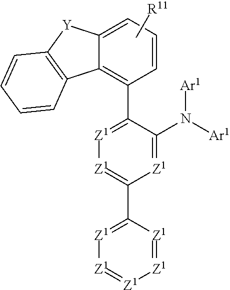 | Formula (I-A-a) |
| 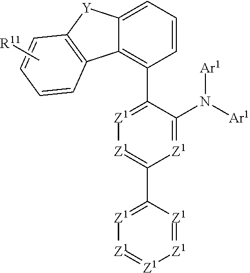 | Formula (I-A-b) |
| 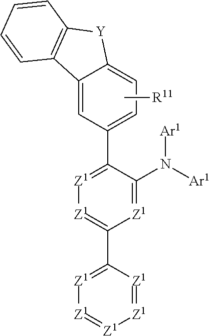 | Formula (I-B-a) |
| 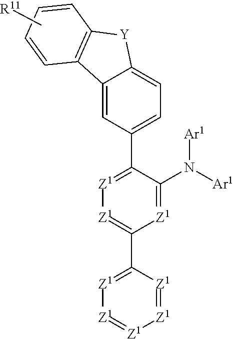 | Formula (I-B-b) |
| 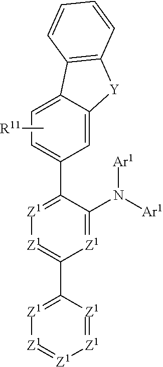 | Formula (I-C-a) |
| 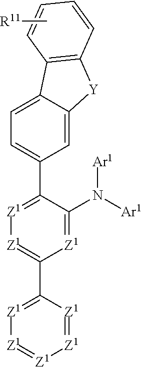 | Formula (I-C-b) |
| 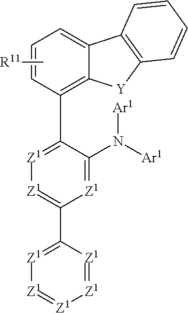 | Formula (I-D-a) |
| 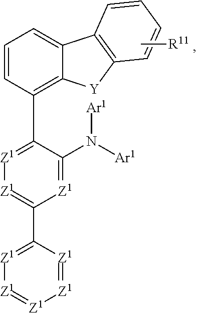 | Formula (I-D-b) |

wherein the variable groups are defined as in claim 1, and where Y is preferably O, and wherein Z¹ is preferably CR¹.

3. The compound according to claim 1, characterized in that exactly one of Z¹¹, Z¹², Z¹³, Z¹⁴, Z⁵, Z¹⁶, Z¹⁷, and Z¹⁸ per formula is CR¹¹.

4. The compound according to claim 1, characterized in that the bond drawn into the ring comprising Z¹¹, Z¹², Z¹³ and Z¹⁴ is attached to one of Z¹² and Z¹³.

5. The compound according to claim 1, characterized in that Y is O.

6. The compound according to claim 1, characterized in that groups Ar¹ are selected, identically or differently, from phenyl, biphenyl, terphenyl, and quaterphenyl, which each bear no substituents, and from fluorenyl, which is attached in one of its positions 1 to 4, and which is substituted with radicals R².

7. The compound according to claim 1, characterized in that one of the following cases a) and b) applies: a) both groups Ar¹ are biphenyl, which bears no substituents; and b) both groups Ar¹ are fluorenyl, which is attached in one of its positions 1 to 4 and which is substituted with radicals R³.

8. The compound according to claim 1, characterized in that R¹ is selected, identically or differently, from H, D, straight-chain alkyl groups having 1 to 20 C atoms, branched or cyclic alkyl groups having 3 to 20 C atoms, aromatic ring systems having 6 to 40 aromatic ring atoms, which are substituted by radicals R³, and heteroaromatic ring systems having 5 to 40 aromatic ring atoms, which are substituted by radicals R³; and where R¹ is preferably selected from H and D; and where most preferably R¹ is H.

9. The compound according to claim 1, characterized in that R¹¹ is selected, identically or differently, from aromatic ring systems having 6 to 24 aromatic ring atoms, which are substituted by radicals R³, most preferably R¹¹ is selected from phenyl, biphenyl, terphenyl, and naphthyl, which are substituted by radicals R³.

10. The compound according to claim 1, characterized in that R² which are on the aromatic rings of the fluorenyl group which is Ar¹ are, identically or differently, selected from H, D, F, straight-chain alkyl groups having 1 to 20 C atoms, and branched or cyclic alkyl groups having 3 to 20 C atoms; and R² which are on the bridgehead aliphatic carbon atom of the fluorenyl group which is Ar¹ are, identically or differently, selected from F, straight-chain alkyl groups having 1 to 20 C atoms, branched or cyclic alkyl groups having 3 to 20 C atoms, and aromatic ring systems having 6 to 40 aromatic ring atoms, which are substituted by radicals R³.

11. The compound according to claim 2, characterized in that the compound fulfils the following conditions:
the compound conforms to one of formulae (I-B-a), (I-B-b), (I-C-a) and (I-C-b);
Y is O;
Z¹ is CR¹;
Z¹¹, Z¹², Z¹³, Z¹⁴ is each selected, identically or differently, from CR¹ and CR¹¹, if the bond drawn into the ring comprising Z¹¹, Z¹², Z¹³ and Z¹⁴ is not attached to it, and Z¹¹, Z¹², Z¹³, Z¹⁴ is C, if the bond drawn into the ring comprising Z¹¹, Z¹², Z¹³, Z¹⁴ is attached to it;
Z¹⁵, Z¹⁶, Z¹⁷, Z¹⁸ is selected, identically or differently, from CR¹ and CR¹¹;
exactly one of Z¹¹, Z¹², Z¹³, Z¹⁴, Z¹⁵, Z¹⁶, Z¹⁷ and Z¹⁸ is CR¹¹;
Ar¹ is selected, identically or differently, from phenyl, biphenyl, terphenyl, and quaterphenyl, which each bear no substituents, and fluorenyl, which is attached in one of its positions 1 to 4, and which is substituted with radicals R²;
R¹ is selected, identically or differently from H, D, straight-chain alkyl groups having 1 to 20 C atoms, branched or cyclic alkyl groups having 3 to 20 C atoms, aromatic ring systems having 6 to 40 aromatic ring atoms, which are substituted by radicals R³, and heteroaromatic ring systems having 5 to 40 aromatic ring atoms, which are substituted by radicals R³;
R¹¹ is selected, identically or differently, from phenyl, biphenyl, terphenyl, and naphthyl, which are substituted by radicals R³, where R³ is selected identically or differently from H, D, F, straight-chain alkyl groups having 1 to 20 C atoms, and branched or cyclic alkyl groups having 3 to 20 C atoms;
R² is selected identically or differently from H, D, F, straight-chain alkyl groups having 1 to 20 C atoms, branched or cyclic alkyl groups having 3 to 20 C atoms, aromatic ring systems having 6 to 40 aromatic ring atoms, which are substituted by radicals R³, and heteroaromatic ring systems having 5 to 40 aromatic ring atoms, which are substituted by radicals R³;
R³ is selected, identically or differently, from H, D, F, CN, Si(R⁴)₃, N(R⁴)₂, straight-chain alkyl or alkoxy groups having 1 to 20 C atoms, branched or cyclic alkyl or alkoxy groups having 3 to 20 C atoms, aromatic ring systems having 6 to 40 aromatic ring atoms, and heteroaromatic ring systems having 5 to 40 aromatic ring atoms; where the said alkyl and alkoxy groups and the said aromatic and heteroaromatic ring systems are substituted by radicals R⁴; and
R⁴ is selected, identically or differently, from H, D, F, CN, alkyl groups having 1 to 20 C atoms, aromatic ring systems having 6 to 40 C atoms, and heteroaromatic ring systems having 5 to 40 aromatic ring atoms; where the said alkyl groups, aromatic ring systems and heteroaromatic ring systems may be substituted by one or more radicals selected from F and CN.

12. A process for preparing a compound according to claim 1, comprising, in a first step, reacting a biphenyl derivate which bears two reactive groups, where one of the two reactive groups is present in the 3-position of the biphenyl derivate, and the other is present in the 4-position of the biphenyl derivate, with a dibenzofuranyl or dibenzothiophenyl derivate which bears a reactive group on one of its aromatic cores and which bears an aromatic or heteroaromatic group as substituent, in a Suzuki coupling reaction, wherein the reactive group in the 4-position reacts in the coupling reaction.

13. A formulation comprising at least one compound according to claim 1 and at least one solvent.

14. An electronic device, comprising at least one compound according to claim 1.

15. The electronic device according to claim 14, characterized in that it is an organic electroluminescent device and comprises an anode, cathode and at least one emitting layer, and in that the compound is present in a hole-transporting layer or in an emitting layer of the device.
